SEQUENCE LISTING (54 sequences)

SEQ ID NO: 1 - RNA, 4 nt, Artificial Sequence
  Description of Artificial Sequence Synthetic oligonucleotide
cuga

SEQ ID NO: 2 - RNA, 22 nt, Artificial Sequence
  Description of Artificial Sequence Synthetic oligonucleotide
  modified_base (1)..(16): g, a, c or u or modified versions thereof
  See specification as filed for detailed description of substitutions and preferred embodiments
nnnnnnnnnnnnnnnnucaggg

SEQ ID NO: 3 - DNA, 25 bp, Artificial Sequence
  Description of Artificial Sequence Synthetic oligonucleotide
  Description of Combined DNA/RNA Molecule Synthetic oligonucleotide
cugaugacacuggcuaguucaccut

SEQ ID NO: 4 - RNA, 27 nt, Artificial Sequence
  Description of Artificial Sequence Synthetic oligonucleotide
aaggugaacuagccagugucaucaggg

SEQ ID NO: 5 - RNA, 25 nt, Artificial Sequence
  Description of Artificial Sequence Synthetic oligonucleotide
cugagguuacugaaucuaacaauga

SEQ ID NO: 6 - RNA, 27 nt, Artificial Sequence
  Description of Artificial Sequence Synthetic oligonucleotide
acuuuguuagauucaguaaccucaggg

SEQ ID NO: 7 - DNA, 15 bp, Artificial Sequence
  Description of Artificial Sequence Synthetic oligonucleotide
  Description of Combined DNA/RNA Molecule Synthetic oligonucleotide
cugaugacacuggct

SEQ ID NO: 8 - RNA, 17 nt, Artificial Sequence
  Description of Artificial Sequence Synthetic oligonucleotide
agccagugucaucaggg

SEQ ID NO: 9 - RNA, 25 nt, Artificial Sequence
  Description of Artificial Sequence Synthetic oligonucleotide
cugacaucgucucgcauuuaugagc

SEQ ID NO: 10 - RNA, 27 nt, Artificial Sequence
  Description of Artificial Sequence Synthetic oligonucleotide
gcucauaaaugcgagacgaugucaggg

SEQ ID NO: 11 - DNA, 19 bp, Artificial Sequence
  Description of Artificial Sequence Synthetic oligonucleotide
  Description of Combined DNA/RNA Molecule Synthetic oligonucleotide
acacuggcuaguucacctt

SEQ ID NO: 12 - RNA, 21 nt, Artificial Sequence
  Description of Artificial Sequence Synthetic oligonucleotide
aaggugaacuagccaguguca

SEQ ID NO: 13 - DNA, 25 bp, Artificial Sequence
  Description of Artificial Sequence Synthetic oligonucleotide
  Description of Combined DNA/RNA Molecule Synthetic oligonucleotide
cugaugacacuggcuaguucacctt

SEQ ID NO: 14 - RNA, 27 nt, Artificial Sequence
  Description of Artificial Sequence Synthetic oligonucleotide
aaggugaacuagccagugucaucaggg

SEQ ID NO: 15 - DNA, 19 bp, Artificial Sequence
  Description of Artificial Sequence Synthetic oligonucleotide
  Description of Combined DNA/RNA Molecule Synthetic oligonucleotide
cugaugacacuggcuagtt

SEQ ID NO: 16 - RNA, 21 nt, Artificial Sequence
  Description of Artificial Sequence Synthetic oligonucleotide
aacuagccagugucaucaggg

SEQ ID NO: 17 - DNA, 25 bp, Artificial Sequence
  Description of Artificial Sequence Synthetic oligonucleotide
  Description of Combined DNA/RNA Molecule Synthetic oligonucleotide
cugaugacacuggcuaguucacctt

SEQ ID NO: 18 - RNA, 27 nt, Artificial Sequence
  Description of Artificial Sequence Synthetic oligonucleotide
aaggugaacuagccagugucaucaggg

SEQ ID NO: 19 - RNA, 25 nt, Artificial Sequence
  Description of Artificial Sequence Synthetic oligonucleotide
cugacaucacugcgcauuuaugagc

SEQ ID NO: 20 - RNA, 27 nt, Artificial Sequence
  Description of Artificial Sequence Synthetic oligonucleotide
gcucauaaaugcgcagugaugucaggg

SEQ ID NO: 21 - DNA, 25 bp, Artificial Sequence
  Description of Artificial Sequence Synthetic oligonucleotide
  Description of Combined DNA/RNA Molecule Synthetic oligonucleotide
ucagugacacuggcuaguucacctt

SEQ ID NO: 22 - RNA, 27 nt, Artificial Sequence
  Description of Artificial Sequence Synthetic oligonucleotide
aaggugaacuagccagugucacugacc

SEQ ID NO: 23 - DNA, 21 bp, Artificial Sequence
  Description of Artificial Sequence Synthetic oligonucleotide
  Description of Combined DNA/RNA Molecule Synthetic oligonucleotide
ugacacuggcuaguucacctt

SEQ ID NO: 24 - RNA, 23 nt, Artificial Sequence
  Description of Artificial Sequence Synthetic oligonucleotide
aaggugaacuagccagugucauc

SEQ ID NO: 25 - DNA, 25 bp, Artificial Sequence
  Description of Artificial Sequence Synthetic oligonucleotide
  Description of Combined DNA/RNA Molecule Synthetic oligonucleotide
cugaugacacuggcuaguucacctt

SEQ ID NO: 26 - RNA, 25 nt, Artificial Sequence
  Description of Artificial Sequence Synthetic oligonucleotide
aaggugaacuagccagugucaucag

SEQ ID NO: 27 - DNA, 25 bp, Artificial Sequence
  Description of Artificial Sequence Synthetic oligonucleotide
  Description of Combined DNA/RNA Molecule Synthetic oligonucleotide
cugaugacacuggcuaguucacctt

SEQ ID NO: 28 - RNA, 27 nt, Artificial Sequence
  Description of Artificial Sequence Synthetic oligonucleotide
aaggugaacuagccagugucaucagcc

SEQ ID NO: 29 - DNA, 25 bp, Artificial Sequence
  Description of Artificial Sequence Synthetic oligonucleotide
  Description of Combined DNA/RNA Molecule Synthetic oligonucleotide
ucagugacacuggcuaguucacctt

SEQ ID NO: 30 - RNA, 27 nt, Artificial Sequence
  Description of Artificial Sequence Synthetic oligonucleotide
aaggugaacuagccagugucacugagg

SEQ ID NO: 31 - DNA, 25 bp, Artificial Sequence
  Description of Artificial Sequence Synthetic oligonucleotide
  Description of Combined DNA/RNA Molecule Synthetic oligonucleotide
ugcaugacacuggcuaguucacctt

SEQ ID NO: 32 - RNA, 27 nt, Artificial Sequence
  Description of Artificial Sequence Synthetic oligonucleotide
aaggugaacuagccagugucaugcagg

SEQ ID NO: 33 - DNA, 25 bp, Artificial Sequence
  Description of Artificial Sequence Synthetic oligonucleotide
  Description of Combined DNA/RNA Molecule Synthetic oligonucleotide
ucgaugacacuggcuaguucacctt

SEQ ID NO: 34 - RNA, 27 nt, Artificial Sequence
  Description of Artificial Sequence Synthetic oligonucleotide
aaggugaacuagccagugucaucgagg

SEQ ID NO: 35 - DNA, 18 bp, Artificial Sequence
  Description of Artificial Sequence Synthetic oligonucleotide
  Description of Combined DNA/RNA Molecule Synthetic oligonucleotide
cugaugacacuggcuagt

SEQ ID NO: 36 - RNA, 20 nt, Artificial Sequence
  Description of Artificial Sequence Synthetic oligonucleotide
acuagccagugucaucaggg

SEQ ID NO: 37 - RNA, 17 nt, Artificial Sequence
  Description of Artificial Sequence Synthetic oligonucleotide
cugaugacacuggcuag

SEQ ID NO: 38 - RNA, 19 nt, Artificial Sequence
  Description of Artificial Sequence Synthetic oligonucleotide
cuagccagugucaucaggg

SEQ ID NO: 39 - DNA, 16 bp, Artificial Sequence
  Description of Artificial Sequence Synthetic oligonucleotide
  Description of Combined DNA/RNA Molecule Synthetic oligonucleotide
cugaugacacuggcta

SEQ ID NO: 40 - RNA, 18 nt, Artificial Sequence
  Description of Artificial Sequence Synthetic oligonucleotide
uagccagugucaucaggg

SEQ ID NO: 41 - RNA, 20 nt, Artificial Sequence
  Description of Artificial Sequence Synthetic oligonucleotide
  modified_base (5)..(20): g, a, c or u or modified versions thereof
  See specification as filed for detailed description of substitutions and preferred embodiments
cugannnnnnnnnnnnnnnn

SEQ ID NO: 42 - DNA, 18 bp, Artificial Sequence
  Description of Artificial Sequence Synthetic oligonucleotide
  Description of Combined DNA/RNA Molecule Synthetic oligonucleotide
  modified_base (5)..(8): g, a, c, u or t
  modified_base (13)..(18): g, a, c, u or t
  See specification as filed for detailed description of substitutions and preferred embodiments
cugannnnacugnnnnnn

SEQ ID NO: 43 - DNA, 20 bp, Artificial Sequence
  Description of Artificial Sequence Synthetic oligonucleotide
  Description of Combined DNA/RNA Molecule Synthetic oligonucleotide
  modified_base (1)..(6): g, a, c, u or t
  modified_base (11)..(14): g, a, c, u or t
  See specification as filed for detailed description of substitutions and preferred embodiments
nnnnnncagunnnnucaggg

SEQ ID NO: 44 - DNA, 20 bp, Artificial Sequence
  Description of Artificial Sequence Synthetic oligonucleotide
gcctggaccacaagtttgac

SEQ ID NO: 45 - DNA, 20 bp, Artificial Sequence
  Description of Artificial Sequence Synthetic oligonucleotide
cagtacgagggtcttaaagt

SEQ ID NO: 46 - DNA, 28 bp, Artificial Sequence
  Description of Artificial Sequence Synthetic oligonucleotide
ctcttgtagatctgttctctaaacgaac

SEQ ID NO: 47 - DNA, 20 bp, Artificial Sequence
  Description of Artificial Sequence Synthetic oligonucleotide
gcgtaatgcaaaccacctgg

SEQ ID NO: 48 - DNA, 20 bp, Artificial Sequence
  Description of Artificial Sequence Synthetic oligonucleotide
ccaaggccaaccgtgaaaag

SEQ ID NO: 49 - DNA, 20 bp, Artificial Sequence
  Description of Artificial Sequence Synthetic oligonucleotide
atggctacgtacatggctgg

SEQ ID NO: 50 - DNA, 20 bp, Artificial Sequence
  Description of Artificial Sequence Synthetic oligonucleotide
ggtccaccaaacgtaatgcg

SEQ ID NO: 51 - RNA, 22 nt, Artificial Sequence
  Description of Artificial Sequence Synthetic oligonucleotide
uaaaugcgagacgaugucaggg

SEQ ID NO: 52 - RNA, 23 nt, Artificial Sequence
  Description of Artificial Sequence Synthetic oligonucleotide
ugaacuagccagugucaucaggg

SEQ ID NO: 53 - DNA, 18 bp, Artificial Sequence
  Description of Artificial Sequence Synthetic oligonucleotide
  Description of Combined DNA/RNA Molecule Synthetic oligonucleotide
  modified_base (5)..(18): g, a, c, u or t
  See specification as filed for detailed description of substitutions and preferred embodiments
cugannnnnnnnnnnnnn

SEQ ID NO: 54 - DNA, 20 bp, Artificial Sequence
  Description of Artificial Sequence Synthetic oligonucleotide
  Description of Combined DNA/RNA Molecule Synthetic oligonucleotide
  modified_base (1)..(14): g, a, c, u or t
  See specification as filed for detailed description of substitutions and preferred embodiments
nnnnnnnnnnnnnnucaggg

(19) United States
(12) Patent Application Publication
(10) Pub. No.: US 2023/0242918 A1
(43) Pub. Date: Aug. 3, 2023

(54) INTERFERON- INDUCING OLIGONUCLEOTIDE DUPLEXES AND METHODS OF USE

(71) Applicant: PRESIDENT AND FELLOWS OF HARVARD COLLEGE, Cambridge, MA (US)

(72) Inventors: Longlong SI, Cambridge, MA (US); Haiqing BAI, Cambridge, MA (US); Donald E. INGBER, Cambridge, MA (US); Crystal Yuri OH, Cambridge, MA (US); Rachelle PRANTIL-BAUN, Cambridge, MA (US)

(73) Assignee: PRESIDENT AND FELLOWS OF HARVARD COLLEGE, Cambridge, MA (US)

(21) Appl. No.: 17/926,748

(22) PCT Filed: May 21, 2021
(86) PCT No.: PCT/US2021/033617

§ 371 Date: Nov. 21, 2022

Related U.S. Application Data
(60) Provisional application No. 63/029,199, filed on May 22, 2020.
(60) Provisional application No. 63/082,742, filed on Sep. 24, 2020.

(51) Int. Cl. C12N 15/117; A61K 45/06; A61K 31/713; A61P 31/16; A61P 31/14; A61K 35/16; A61K 39/39

(52) U.S. Cl. CPC C12N 15/117; A61K 45/06; A61K 31/713; A61P 31/16; A61P 31/14; A61K 35/16; A61K 39/39; C12N 2310/14; C12N 2310/31; A61K 2039/55561

(57) ABSTRACT

Described herein are compositions and methods for inducing Type I interferon production. The compositions described comprise immunostimulatory oligonucleotide duplexes including a 5′ terminal monophosphate-CUGA-3′ (SEQ ID NO. 1) sequence. Compositions comprising the immunostimulatory oligonucleotide duplexes described can be used for the treatment of diseases or disorders that respond to interferons.

### CROSS-REFERENCE TO RELATED APPLICATIONS

This application is a 35 U.S.C. § 371 National Phase Entry Application of International Application No. PCT/US2021/033617 filed May 21, 2021, which designates the U.S. and claims benefit under 35 U.S.C. § 119(e) of U.S. Provisional Application No. 63/029,199 filed May 22, 2020 and U.S. Provisional Application No. 63/082,742 filed Sep. 24, 2020 the contents of which are incorporated herein by reference in their entireties.

### GOVERNMENT SUPPORT

This invention was made with government support under HL141797 awarded by the National Institutes of Health and under W911NF-12-2-0036 and W911NF-16-C-0050 awarded by the U.S. Army. The government has certain rights in the invention.

### SEQUENCE LISTING

The instant application contains a Sequence Listing which has been submitted electronically in ASCII format and is hereby incorporated by reference in its entirety. Said ASCII copy, created on Jun. 30, 2021, is named 002806-097470WOPT_SL.txt and is 16,110 bytes in size.

### TECHNICAL FIELD

The technology described herein relates to compositions and methods for immunostimulation.

### BACKGROUND

Pathogenic infections trigger a complex regulatory system of innate and adaptive immune responses designed to defend against the pathogen in the host organism. One of the many responses to the pathogen invasion, e.g., viral, bacterial, fungal or parasitic infection, is the induction of interferon (IFN) production, a pleiotropic group of cytokines that play a critical role in human immune responses by ‘interfering’ with pathogen activity, e.g., viral replication, among others. The increasing incidence of pandemic viruses, such as influenza, MERS, SARS, and now SARS-CoV-2, requires development of new broad-spectrum therapies that inhibit infection by many different types of viruses and pathogens.

### SUMMARY

The compositions and methods described herein relate, in part, to the discovery of oligonucleotide duplexes that induce interferon production.

In one aspect, described herein is an immunostimulatory oligonucleotide duplex comprising SEQ ID NO:1 at a 5′ end.

In one embodiment of this or any other aspect, the oligonucleotide duplex is RNA.

In another embodiment of this or any other aspect, the oligonucleotide duplex comprises a 5′-monophosphate group.

In another embodiment of this or any other aspect, there is no modification to the 5′ terminal sequence (SEQ ID NO: 1).

In another embodiment of this or any other aspect, the oligonucleotide duplex is at least 20 nucleobases in length.

In another embodiment of this or any other aspect, the oligonucleotide duplex is double stranded RNA.

In another embodiment of this or any other aspect, the oligonucleotide duplex is sufficient to induce interferon (IFN) production in a cell contacted with the duplex.

In another embodiment of this or any other aspect, the oligonucleotide duplex activates the RIG-I-IRF3 pathway.

In another embodiment of this or any other aspect, the oligonucleotide duplex reduces a viral titer in a cell or cell population contacted with the duplex.

In another embodiment of this or any other aspect, the oligonucleotide duplex increases STAT1 and STAT2 in a cell contacted by the duplex.

The immunostimulatory oligonucleotide duplexes as described herein can be used to treat or assist in the treatment of any disease or disorder that can benefit from the induction of an interferon response. Such diseases or disorders include viral infection, as well as infection with bacterial, fungal or parasitic pathogens, as well as cancers and autoimmune diseases that benefit from interferon induction. Thus, disclosed herein are methods of treating viral, bacterial, fungal or parasitic infection comprising administering an immunostimulatory oligonucleotide duplex as described herein to a subject in need thereof. Similarly, also disclosed herein are methods of treating cancer or autoimmune disease comprising administering an immunostimulatory oligonucleotide duplex as described herein to a subject in need thereof.

In another aspect, described herein is a method of inducing an anti-viral response in a subject, the method comprising administering to a subject in need thereof an immunostimulatory oligonucleotide duplex as described herein.

In one embodiment of this or any other aspect, the subject in need thereof has a viral infection, or is at risk of having a viral infection.

In another embodiment of this or any other aspect, the method further comprises, prior to administering, a step of diagnosing the subject as having a viral infection or being at risk of having a viral infection.

In another embodiment of this or any other aspect, the method further comprises, prior to administering, a step of receiving results of an assay that diagnoses the subject as having a viral infection or as being at risk of having a viral infection.

In another embodiment of this or any other aspect, the viral infection is caused by a virus selected from the group consisting of: John Cunningham virus, measles virus, Lymphocytic choriomeningitis virus, arbovirus, rabies virus, rhinovirus, parainfluenza virus, respiratory syncytial virus, herpes simplex virus, herpes simplex type 1, herpes simplex type 2, human herpesvirus 6, adenovirus, cytomegalovirus, Epstein-Barr virus, mumps virus, influenza virus type A, influenza virus type B, coronavirus, SARS coronavirus, SARS-CoV-2 virus, coxsackie A virus, coxsackie B virus, poliovirus, HTLV-1, hepatitis virus types A, B, C, D, and E, varicella zoster virus, smallpox virus, molluscum contagiosum, human papillomavirus, parvovirus B19, rubella virus, human immunodeficiency virus, rotavirus, norovirus, astrovirus, ebola virus, Marburg virus, dengue virus (DENV), and Zika virus.

In another embodiment of this or any other aspect, the viral infection is an infection of a tissue selected from the group consisting of: central nervous system tissue, eye tissue, upper respiratory system tissue, lower respiratory system tissue, lung tissue, kidney tissue, bladder tissue, spleen tissue, cardiac tissue, gastrointestinal tissue, epidermal tissue, reproductive tissue, nasal cavity tissue, larynx tissue, trachea tissue, bronchi tissue, oral cavity tissue, blood tissue, and muscle tissue.

In another embodiment of this or any other aspect, the administration is systemic.

In another embodiment of this or any other aspect, the administration is local at a site of infection.

In another embodiment of this or any other aspect, the method further comprises administering at least one additional therapeutic.

In another embodiment of this or any other aspect, the at least one additional therapeutic is an anti-viral therapeutic.

In another aspect, described herein is a method of treating an influenza infection in a subject, the method comprising administering to a subject having an influenza infection an immunostimulatory oligonucleotide duplex as described herein.

In one embodiment of this or any other aspect, the influenza infection is an influenza A infection, or an influenza B infection.

In another embodiment of this or any other aspect, the method further comprises administering at least one additional anti-viral therapeutic.

In another aspect, described herein is a method of treating a coronavirus disease in a subject, the method comprising administering to a subject having a coronavirus disease an immunostimulatory oligonucleotide duplex as described herein.

In one embodiment of this or any other aspect, the coronavirus disease is COVID-19.

In another embodiment of this or any other aspect, the method further comprises administering at least one additional anti-viral therapeutic.

In another embodiment of this or any other aspect, the method further comprises administering plasma obtained from a subject that has recovered from the coronavirus disease.

In another aspect, described herein is a method of increasing the efficacy of an anti-viral therapeutic, the method comprising administering an immunostimulatory oligonucleotide duplex as described herein and at least one anti-viral therapeutic.

In one embodiment of this or any other aspect, the anti-viral therapeutic is selected from the group consisting of: Abacavir, Acyclovir (Aciclovir), Adefovir, Amantadine, Ampligen, Amprenavir (Agenerase), Amodiaquine, Apilimod, Arbidol, Atazanavir, Atripla, Atovaquone, Balavir, Baloxavir marboxil (Xofluza®), Biktarvy Boceprevir (Victrelis®), Cidofovir, Clofazimine, Clomifene, Clofazamine, Cobicistat (Tybost®), Combivir (fixed dose drug), Daclatasvir (Daklinza®), Darunavir, Delavirdine, Descovy, Didanosine, Docosanol, Dolutegravir, Doravirine (Pifeltro®), Ecoliever, Edoxudine, Efavirenz, Elvitegravir, Emtricitabine, Enfuvirtide, Entecavir, Etravirine (Intelence®), Famciclovir, Favipiravir, Fenofibrate, Fomivirsen, Fosamprenavir, Foscamet, Fosfonet, Fusion inhibitor, Ganciclovir (Cytovene®), Ibacitabine, Ibalizumab (Trogarzo®), Idoxuridine, Imiquimod, Imunovir, Indinavir, Inosine, Integrase inhibitor, Interferon type I, Interferon type II, Interferon type III, Interferon, Ivermectin, Lamivudine, Lasalocid, Letermovir (Prevymis®), Lopinavir, Loviride, Mannose Binding Lectin, Maraviroc, Methisazone, Moroxydine, Nafamostat, Nelfinavir, Nevirapine, Nexavir®, Nilotinib, Nitazoxanide, Norvir, Nucleoside analogues, Oseltamivir (Tamiflu®), Pazopanib, Peginterferon alfa-2a, Peginterferon alfa-2b, Penciclovir, Peramivir (Rapivab®), Pleconaril, Podophyllotoxin, Protease inhibitor (pharmacology), Pyonaridine, Pyramidine, Raltegravir, Remdesivir, Reverse transcriptase inhibitor, Ribavirin, Rilpivirine (Edurant®), Rimantadine, Ritonavir, Saquinavir, Simeprevir (Olysio®), Sofosbuvir, Stavudine, Synergistic enhancer (antiretroviral), Tafenoquine, Telaprevir, Telbivudine (Tyzeka®), Tenofovir alafenamide, Tenofovir disoproxil, Tenofovir, Toremifene, Tipranavir, Trifluridine, Trizivir, Tromantadine, Truvada, Valaciclovir (Valtrex), Valganciclovir, Vermurafenib, Venetoclax, Vicriviroc, Vidarabine, Viramidine, Zalcitabine, Zanamivir (Relenza®), and Zidovudine.

In another embodiment of this or any other aspect, the immunostimulatory oligonucleotide duplex and the at least one antiviral therapeutic are administered at substantially the same time.

In another embodiment of this or any other aspect, the immunostimulatory oligonucleotide duplex and the at least one antiviral therapeutic are administered at different time points

In another aspect, described herein is a pharmaceutical composition comprising an immunostimulatory oligonucleotide duplex as described herein and a pharmaceutically acceptable carrier.

In one embodiment of this or any other aspect, the composition is formulated for airway administration. In another embodiment of this or any other aspect, the composition is formulated for aerosol administration, nebulizer administration, or tracheal lavage administration.

In another aspect, described herein is a pharmaceutical composition comprising an immunostimulatory oligonucleotide duplex described herein and at least one anti-viral therapeutic.

In one embodiment of this or any other aspect, the composition is formulated for intravenous, intramuscular, intraperitoneal, subcutaneous, or intrathecal administration.

In another aspect, described herein is a method of inducing interferon (IFN) production, the method comprising administering to a subject in need thereof an immunostimulatory oligonucleotide duplex as described herein, or a pharmaceutical composition comprising such duplex as described herein, whereby IFN production is increased following administration.

In one embodiment of this or any other aspect, IFN production is the production of type I IFN, type II IFN, or type III IFN.

In another embodiment of this or any other aspect, IFN production is the production of type I IFN, including one or more of IFN-α, IFN-β, IFN-ε, IFN-κ and IFN-ω.

In another embodiment of this or any other aspect, the type II IFN is IFN-γ

In another embodiment of this or any other aspect, increased IFN production increases cellular resistance to a viral infection.

In another embodiment of this or any other aspect, the subject in need thereof has an IFN-associated disease, or is at risk of having an IFN-associated disease.

In another embodiment of this or any other aspect, the method further comprises, prior to administering, a step of diagnosing a subject as having an IFN-associated disease or being at risk of having an IFN-associated disease.

In another embodiment of this or any other aspect, the method further comprises, prior to administering, receiving the results of an assay that diagnoses a subject as having an IFN-associated disease or being at risk of an IFN-associated disease.

In another embodiment of this or any other aspect, the IFN-associated disease is a disease involving reduced IFN levels as compared to a reference level.

In another embodiment of this or any other aspect, the IFN-associated disease is a disease involving reduced Type I IFN levels as compared to a reference level.

In another embodiment of this or any other aspect, the IFN-associated disease is selected from the group consisting of a viral infectious disease, a bacterial infectious disease, a fungal infectious disease, a parasitic infectious disease, cancer, and an autoimmune disease.

In another embodiment of this or any other aspect, the method further comprises administering at least one additional therapeutic.

In another embodiment of this or any other aspect, the at least one additional therapeutic is an anti-viral therapeutic, an anti-bacterial therapeutic, an anti-fungal therapeutic, an anti-parasitic therapeutic, an anti-cancer therapeutic, or an anti-autoimmune therapeutic.

In another aspect, described herein is a composition comprising an immunostimulatory oligonucleotide duplex as described herein and at least one anti-bacterial therapeutic. In one embodiment of this aspect, the composition further comprises a pharmaceutically acceptable carrier.

In another aspect, described herein is a composition comprising an immunostimulatory oligonucleotide duplex as described herein and at least one anti-fungal therapeutic. In one embodiment of this aspect, the composition further comprises a pharmaceutically acceptable carrier.

In another aspect, described herein is a composition comprising an immunostimulatory oligonucleotide duplex as described herein and at least one anti-parasitic therapeutic. In one embodiment of this aspect, the composition further comprises a pharmaceutically acceptable carrier.

In another aspect, described herein is a composition comprising an immunostimulatory oligonucleotide duplex as described herein and at least one anti-cancer therapeutic. In one embodiment of this aspect, the composition further comprises a pharmaceutically acceptable carrier.

In another aspect, described herein is a composition comprising an immunostimulatory oligonucleotide duplex as described herein and at least one therapeutic for the treatment of autoimmune disease. In one embodiment of this aspect, the composition further comprises a pharmaceutically acceptable carrier.

In another aspect, described herein is an immunostimulatory oligonucleotide duplex as described herein, conjugated to an antigen or nucleic acid sequence encoding an antigen, e.g., for use as a vaccine.

In another aspect, described herein is a composition comprising an immunostimulatory oligonucleotide duplex as described herein and a vaccine.

In another aspect, described herein is a composition comprising an immunostimulatory oligonucleotide duplex as described herein and a nanoparticle.

In another aspect, described herein is a nanoparticle comprising an immunostimulatory oligonucleotide duplex as described herein. In one embodiment of any of the aspects, the nanoparticle is a lipid nanoparticle.

In another aspect, described herein is a method of vaccinating, the method comprising administering to a subject in need thereof an immunostimulatory oligonucleotide duplex as described herein. In one embodiment of this or any other aspect, the immunostimulatory oligonucleotide duplex is administered with an antigen or nucleic acid sequence encoding an antigen. In another embodiment of this or any other aspect, the antigen or nucleic acid encoding the antigen is conjugated to the immunostimulatory oligonucleotide duplex.

In another aspect, described herein is a method of increasing the efficacy of a vaccine, the method comprising administering to a subject in need thereof an immunostimulatory oligonucleotide duplex as described herein. In one embodiment of this or any other aspect, the immunostimulatory oligonucleotide duplex is administered with an antigen or nucleic acid sequence encoding an antigen.

In another embodiment of this or any other aspect, the antigen or nucleic acid encoding the antigen is conjugated to the immunostimulatory oligonucleotide duplex.

### Definitions

As used herein, an “oligonucleotide duplex” encompasses two separate strands of ribonucleic acid that hybridize through the formation of complementary base pairs to form a duplex under physiologically relevant conditions of temperature and ionic strength. The term oligonucleotide duplex also encompasses a single strand that includes self-complementary sequences that permit hybridization to form a duplex under similar conditions. Duplexes formed from a single strand can include a hairpin structure that folds back on itself with few non-hybridized nucleotides at the transition from one strand of the duplex to the other, or a hairpin loop or stem loop structure that includes a more pronounced loop of non-hybridized nucleotides between the hybridized sequences. Immunostimulatory oligonucleotide duplexes as described herein will have a duplexed length of 20 nucleotides or more, not including single stranded overhang (generally GG or a modified form thereof). While a minimum length of 20 nucleotides of duplexed sequence, including the 5′-terminus monophosphate-CUGA-3′ (SEQ ID NO: 1) duplexed sequence, has been determined for immunostimulatory activity of the oligonucleotide duplexes described herein, it is contemplated that a degree of mismatch can be tolerated within the remaining minimum 16 nucleotide length duplex, such that, for example, at least 11 of the remaining 16 nucleotides must be complementary, e.g., at least 11 of the 16, at least 12 of the 16, at least 13 of the 16, at least 14 of the 16, at least 15 of the 16 or all of the at least 16 remaining nucleotides are complementary. Where there is one or more mismatch, it is anticipated that mismatch(es) will be better tolerated if located in the interior of the 20 nucleotide sequence that forms a duplex—i.e., a stretch of nucleotides at both ends are fully complementary, and it is also anticipated that where there are more than one mismatch within the sequence, contiguous mismatches may be less favorable. It is also contemplated that where there is one or more mismatch, a relatively higher GC content in the remaining nucleotides may help offset any relative disadvantage of the mismatch. The same principles would apply for mismatches where the duplex region is greater than 20 nucleotides in length.

As used herein, the term “RNA” refers to ribonucleic acid, which as typically transcribed in nature comprises the purine nucleobases adenine and guanine and the pyrimidine nucleobases cytosine and uracil. RNA oligonucleotides described herein can include modified nucleobases or modifications to the ribose-phosphate backbone that, for example, enhance stability or resistance to degradation.

Examples of such modifications are discussed herein below or known in the art. In one embodiment of any of the aspects described herein, the modification is not removal of the 2′ hydroxyl that distinguishes RNA from deoxyribonucleic acid.

As used herein, the phrase “oligonucleotide duplex comprises a 5′ monophosphate group” means that the monophosphate is on the 5′-terminal C or analogue or modified form thereof of the sequence 5′-CUGA-3′ (SEQ ID NO: 1) comprised by the immunostimulatory oligonucleotide duplexes as described herein.

The terms “increase”, “enhance”, or “activate” are all used herein to mean an increase by a reproducible statistically significant amount. In some embodiments, the terms “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, a 20 fold increase, a 30 fold increase, a 40 fold increase, a 50 fold increase, a 6 fold increase, a 75 fold increase, a 100 fold increase, etc. or any increase between 2-fold and 10-fold or greater as compared to an appropriate control. In the context of a marker, an “increase” is a reproducible statistically significant increase in such level.

The term “decrease”, “reduced”, “reduction”, or “inhibit” are all used herein to mean a decrease by a statistically significant amount. In some embodiments, “decrease”, “reduced”, “reduction”, or “inhibit” typically means a decrease by at least 10% as compared to an appropriate control (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to an appropriate control.

As used herein, a “reference level” refers to a normal, otherwise unaffected cell population or tissue (e.g., a biological sample obtained from a healthy subject, or a biological sample obtained from the subject at a prior time point, e.g., a biological sample obtained from a patient prior to being diagnosed with interferon-mediated disease, or a biological sample that has not been contacted with a composition disclosed herein).

As used herein, an “appropriate control” refers to an untreated, otherwise identical cell or population (e.g., a patient who was not administered an agent described herein, or was administered by only a subset of compositions described herein, as compared to a non-control cell).

As used herein, the term “induces interferon production” or “increases interferon production” means that interferon production is increased by at least three-fold following administration of an immunostimulatory oligonucleotide duplex as described herein or following contacting of a cell, population of cells, tissue or organism with such immunostimulatory oligonucleotide duplex. In some embodiments, an increase in interferon production can be at least four-fold, at least five-fold, at least 10-fold, at least 15-fold, at least 20-fold or more. Interferon production can be measured, for example, by immunoassay (e.g., ELISA, immunoprecipitation, etc.), biological reporter assay or other assays as known in the art.

As used herein, an “interferon associated disease or disorder” or a disease or disorder associated with interferon(s)” is a disease or disorder treatable by administering an interferon, or by inducing production of an interferon.

As used herein, the term “reduce a viral titer” or “reduces viral titer” means that the number of infectious viral particles in a sample, e.g., a serum, blood or tissue sample, or in a cell culture supernate, is reduced by at least 10% by treatment of a subject or a cell culture with an immunostimulatory oligonucleotide duplex as described herein.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with, a disease or disorder. The term “treating” includes reducing or alleviating at least one adverse effect or symptom of a condition, disease or disorder associated with an infection. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation or at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. The term “treatment” of a disease also includes providing relief from the symptoms or side-effects of the disease (including palliative treatment).

As used herein “preventing” or “prevention” refers to any methodology where the disease state does not occur due to the actions of the methodology (such as, but not limited to, administration of a vaccine which prevents infection or illness due to a pathogen). In one aspect, it is understood that prevention can also mean that the disease is not established to the extent that occurs in untreated controls. Accordingly, prevention of a disease encompasses a reduction in the likelihood that a subject can develop the disease, relative to an untreated subject (e.g. a subject who is not treated with the methods or compositions described herein).

The term “statistically significant” or “significantly” refers to statistical significance and generally means a two standard deviation (2SD) or greater difference.

As used herein the term “comprising” or “comprises” is used in reference to compositions, methods, and respective component(s) thereof, that are essential to the method or composition, yet open to the inclusion of unspecified elements, whether essential or not.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

### BRIEF DESCRIPTION OF THE DRAWINGS

FIG. 1A-1B demonstrates the discovery of duplex RNAs whose treatment decreased influenza virus infection. (FIG. 1A) A549 cells were transfected with duplex RNAs (IDT Inc). 24 h later, cells were infected with influenza A/WSN/33 (H1N1) virus (MOI=0.01). Supernatants were collected for viral titer detection by plaque formation assay. Scramble DisRNAs were used as control. (FIG. 1B) Human airway chips were transfected with duplex RNA1 (IDT Inc). 24 h later, cells were infected with influenza A/WSN/33 (H1N1) virus (MOI=0.01). Samples were collected for viral NP gene detection by RT-qPCR. Scramble duplex RNAs were used as control.

FIG. 2 demonstrates different versions of RNAs containing the common sequence can increase IRF-IFN pathway level in A549 cells. A549 Dual™ (InvivoGen®) cells (10,000 cells/well) were seeded in 96-well plate and transfected with indicated RNAs for 48 h. Then luciferase activity, which represents the activation level of IRF-IFN pathway, was measured using QUANTI-Luc™ kit according to the manufacturer's instructions. The OD value of scramble RNA group was set as 1. Six repeats for each sample.

FIG. 3A-3D shows that duplex RNA1 is a positive regulator of Type I interferon (IFN-1) pathways. (FIG. 3A) Volcano plot of differentially expressed genes (DEGs) from RNA-seq after treatment with duplex RNA1. (FIG. 3B) GO Enrichment analysis for DEGs. (FIG. 3C) Volcano plot of differentiated expressed proteins from TMT mass spectrometry after treatment with duplex RNA1. (FIG. 3D) GO Enrichment analysis of differentiated expressed proteins.

FIG. 4 shows that duplex RNA-1 specifically increases STAT1 and STAT2, which are specific for IFN pathway. A549 cells were transfected with duplex RNA-1 and cultured for 48 h before cell samples were collected for detection of indicated genes by qPCR.

FIG. 5 shows the knockout of IRF3 abolished the effect of duplex RNA1 on IFN-1 pathway. Wild-type HAP1 cells, IRF7-knockout HAP1 cells, or IRF3 knockout HAP1 cells were transfected with duplex RNA1 (IDT Inc) to knock down DGCR5. 48 h later, cells were collected for detection of genes of IFN-1 pathway, including STAT1, IL4L1, TRAIL, IFFI6 and IFN-β1, by RT-qPCR. Scramble duplex RNAs were used as control.

FIG. 6 shows that duplex RNA-1 induces IFN production via affecting phosphorylation of IRF3. A549 cells were transfected with duplex RNA-1 and cultured for 48 h before detection of mRNA level of IRF3 by qPCR or detection of phosphorylation level of IRF3 by immunofluorescence staining.

FIG. 7 shows a table of the RNA oligonucleotides examined in Example 1. FIG. 7 discloses SEQ ID NOS 3-18, respectively, in order of appearance.

FIG. 8 demonstrates that dsRNA-4 increases IFN-β production. Differentiated human primary airway epithelial cells, human primary alveolar epithelial cells or human lung primary microvascular endothelial cells (HMVEC) were transfected with Negative control (NC) or dsRNA-4. qPCR was performed 48 hours later to measure the IFN-beta production.

FIG. 9 demonstrates dsRNA-1 and dsRNA-2 induced high levels of IFN-beta and inhibited native SARS-CoV-2 by approximately 10⁴-fold. ACE2-expressing A549 cells were transfected with indicated dsRNAs. Twenty-four hours post transfection, ACE2-A549s were infected at an MOI 0.05 with SARS-CoV-2 for 48 hours. Cells were harvested in Trizol™, and total RNA was isolated and DNAse-I treated using Zymo RNA Miniprep Kit™ according to the manufacturer's protocol. qPCR was performed to detect the levels of indicated genes. The gene level of low dose dsRNA was set as 1.

FIG. 10A-10D shows evaluation of novel immunostimulatory RNAs. FIG. 10A shows A549 cells were transfected with RNA-A, RNA-B, or a scrambled duplex RNA control, and infected with influenza A/WSN/33 (H1N1) virus (MOI=0.01) 24 hours later. Titers of progeny viruses in medium supernatants collected at 48 h post-infection were determined by quantifying plaque forming units (PFUs); data are shown as % viral infection measured in the cells treated with the control RNA (Data shown are mean±standard deviation; N=3; ***, P<0.001). FIG. 10B shows qPCR analysis of cellular IFN-β and IFN-α RNA levels at 48 h after A549 cells were transfected with RNA-A, RNA-B, or scrambled dsRNA control (N=3). FIG. 10C shows RNA-mediated production kinetics of IFN production in wild-type A549-Dual cells that were transfected with RNA-A, RNA-B, or scramble RNA control measured using a Quanti-Luc assay. OD values from cells transfected with the scrambled RNA control were subtracted as background (N=6). FIG. 10D demonstrates the dose-dependent induction of IFN by RNA-A and RNA-B in A549-Dual cells compared to scrambled RNA control measured at 48 h post-transfection (control OD values were subtracted as background; N=6).

FIGS. 11A and 11B demonstrate the profiling of the effects of RNA-B by RNA-seq and TMT mass spectrometry. A549 cells were transfected with RNA-B or scrambled RNA control, cell lysates were collected at 48 h, and analyzed by RNA-seq (FIG. 11A) or TMT Mass Spec (FIG. 11B). Differentially expressed genes (DEGs) or proteins are shown in volcano plots (top) and GO Enrichment analysis was performed for the DEGs (bottom) (N=3). Plot (top) and GO Enrichment analysis was performed for the differentially expressed proteins (bottom) (N=3).

FIGS. 12A and 12B demonstrate heat maps showing the effects of immunostimulatory RNAs on IFN pathway-relevant gene levels. DEGs from RNA-seq FIG. 12A and differentially expressed proteins from TMT Mass Spec analyses FIG. 12B shown in FIG. 1B and FIG. 11 are presented here as heat maps (gene levels of the scrambled RNA control were set as 1; N=3).

FIG. 13A-13B demonstrates RNA-induced gene expression associated with type I interferon pathway. FIG. 13A demonstrates a Venn diagram showing differentially expressed ISGs from TMT Mass Spec by RNA-A belong to type I or type II interferon stimulated genes. FIG. 13B shows a heat map of qPCR results showing RNA-I preferentially activates type I interferon pathway. A549 cells were transfected with RNA-A or scrambled dsRNA control, collected at 48 hr and analyzed by qPCR (expression levels were normalized to GAPDH; gene levels induced by the RNA control were set as 1; N=3).

FIG. 14 shows a summary of the RNA oligonucleotide sequences examined in EXAMPLES 2-5. FIG. 14 discloses SEQ ID NOS 13-14, 5-6, 19-20, 9-10, 21-34, 15-16, 35-40, 7-8 and 17-18, respectively, in order of appearance.

FIG. 15 summarizes characteristics of the immunostimulatory RNAs. FIG. 15 discloses “5′-CUGA-3′” as SEQ ID NO: 1.

FIG. 16 shows a comparison of immunostimulatory activities of different RNAs noted in FIG. 14. A549-Dual cells were transfected with indicated duplex RNAs for 48 h, and then activation of the IFN pathway was measured by quantifying luciferase reporter activity. Data are shown as fold change relative to the scrambled RNA control (N=6).

FIG. 17A-17F demonstrates that immunostimulatory RNAs induce IFN-I production through RIG-I-IRF3 pathway. FIG. 17A shows wild-type (WT) HAP1 cells, IRF3 knockout HAP1 cells, or IRF7 knockout HAP1 cells were transfected with RNA-A or scrambled RNA control for 48 h, and IFN-β mRNA levels were quantified by qPCR. Data are shown as fold change relative to the scrambled RNA control (N=3). Note that IRF3 knockdown completely abolished the IFN-β response. FIG. 17B shows IRF3 mRNA levels measured in A549 cells transfected with immunostimulatory RNA-D or a scrambled RNA control, as determined by qPCR and 48 h post-transfection (data are shown as fold change relative to the control RNA; N=3). FIG. 17C shows total IRF3 protein and phosphorylated IRF3 detected in A549 cells transfected with RNA-D or scrambled RNA control at 48 h post transfection as detected by Western blot analysis (GAPDH was used as a loading control). FIG. 17D shows immunofluorescence micrographs showing the distribution of phosphorylated IRF3 in A549 cells transfected with RNA-D or scrambled RNA control at 48 h post transfection; (arrowheads, nuclei expressing phosphorylated IRF3. FIG. 17E shows wild-type (WT) A549-Dual cells, RIG-I knockout A549-Dual cells, MDA5 knockout A549-Dual cells, or TLR3 knockout A549 cells were transfected with immunostimulatory RNA-D or a scrambled RNA control and 48 h later, IFN-β expression levels were quantified using the Quanti-Luc assay or qPCR (data are shown as fold change relative to the scrambled RNA control; N=6). Note that RIG-I knockout abolished the ability of the immunostimulatory RNAs to induce IFN-β. FIG. 17F SPR characterization of the binding affinity between cellular RNA sensors (RIG-I, MDA5, and TLR3) and RNA-1, which were immobilized on a streptavidin (SA) sensor chip. Equilibrium dissociation constant (KD), association rate constant (Ka), and dissociation rate constant (Kd) are labeled on the graphs.

FIG. 18 shows that IRF3 knockout abolished the ability of immunostimulatory RNAs to induce IFN-I pathway associated genes. Wild-type (WT) HAP1 cells, IRF3 knockout HAP1 cells, or IRF7 knockout HAP1 cells were transfected with RNA-A or a scrambled RNA control and STAT1, IL4L1, TRAIL, and IFI6 mRNA levels were quantified by qPCR at 48 h post transfection. Data are presented as fold change relative to RNA control (N=3).

FIG. 19 demonstrates that RIG-I knockout abolished the induction effects of the immunostimulatory RNAs on IFN-β. Wild-type (WT) A549-Dual cells, RIG-I knockout A549-Dual cells, MDA5 knockout A549-Dual cells, or TLR3 knockout A549 cells were transfected with RNA-A, RNA-B, RNA-C, or a scrambled RNA control and IFN-β mRNA levels were detected by Quanti-Luc assay in WT, RIG-I KO, and MDA5 KO A549-Dual cells or qPCR in TLR3 KO A549 cells at 48 h post transfection. Data are shown as fold change relative to the scrambled RNA control (N=6).

FIG. 20A-20D show immunostimulatory RNAs induce IFN-β production in differentiated human lung epithelial and endothelial cells in Organ Chips and exhibit broad spectrum inhibition of infection by influenza H3N2, SARS-CoV-2, SARS-CoV-1, MERS-CoV, and HCoV-NL63. FIG. 20A Schematic diagram of a cross-section through the human Lung-on-Chip, which faithfully recapitulate human lung physiology and pathophysiology. FIG. 20B Human Lung Airway and Alveolus Chips were transfected with RNA-1 or scrambled RNA control by perfusion through both channels of the chip and 48 h later, the epithelial and endothelial cells were collected for detection of IFN-β mRNA by qPCR (data are presented as fold change relative to the RNA control;N=3; *, p<0.05; ***, p<0.001). FIG. 20C Effects of treatment with RNA-1 or a scrambled control in the human Lung Airway Chips or human Lung Alveolus Chips infected with influenza A/HK/8/68 (H3N2) (MOI=0.1). Viral load was determined by quantifying the viral NP gene by qPCR in cell lysates at 48 h after infection. Results are shown as fold change relative to RNA control; N=3; *, p<0.05. FIG. 20D Treatment with immunostimulatory duplex RNAs resulted in potent inhibition of multiple potential pandemic viruses, including SARS-CoV-2. Indicated cells were treated with RNA-1, RNA-2, or a scrambled control and infected with influenza A/HK/8/68 (H3N2) (MOI=0.1), SARS-CoV-2 (MOI=0.05), SARS-CoV-1 (MOI=0.01), MERS-CoV (MOI=0.01), and HCoV-NL63 (MOI=0.002), respectively. Viral load was determined by quantifying the viral NP gene for H3N2, and the N gene for SARS-CoV-2 and HCoV-NL63by qPCR in cell lysates, and the viral titers by plaque assay at 48 h after infection. All results are shown as fold change relative to RNA control; N=3; *, p<0.05; *** p<0.001.

FIG. 21 demonstrates that immunostimulatory RNA-mediated production of IFN in ACE2-overexpressing A549 cells. IFN-β and ISG15 levels were detected in cells transfected with RNA-A, RNA-B, or scrambled dsRNA control by qPCR at 48 h post-transfection. The IFN-β or ISG15 level induced by the scrambled dsRNA control was set as 1. Data are shown as fold change relative to the control (N=3).

FIGS. 22A and 22B demonstrate immunostimulatory RNAs are more potent inducers of IFN-β than Poly (I:C) and 5′ppp-dsRNA, but they do not induce production of proinflammatory cytokines. FIG. 22A Comparison of potency of RNA-1, Poly (I:C), and 5′ppp-dsRNA to induce IFN-β at same concentration (2.8 μg/mL) in A549 cells at 48 h post-transfection. Data are shown as fold change relative to a scramble RNA control. (N=3) FIG. 22B Heat map comparing expression changes of inflammatory genes that are induced by Poly (I:C) or RNA-1 and RNA-2 in A549 cells at 48 h post-transfection.

FIG. 23 shows that immunostimulatory RNAs can inhibit influenza infection in Human Lung Epithelial Cells.

FIG. 24 demonstrates that immunostimulatory RNAs can inhibit common cold coronavirus infection in monkey kidney cells.

FIG. 25 demonstrates RNA-A and RNA-B inhibition of SARS-CoV-2 virus in ACE2-overexpressing Human Lung Epithelial Cells

FIG. 26 demonstrates that immunostimulatory RNA inhibits SARS-CoV2 infection in vivo. Induction of Interferon Type I by Duplex dsRNA administered on day −1, 0, and +1 IN is sufficient to significantly reduce viral load in hamsters.

FIG. 27 illustrates Motif 1 and Motif 1 including Motif 2 of the immunostimulatory RNA duplexes described herein. FIG. 27 discloses SEQ ID NOS 53-54 and 42-43, respectively, in order of appearance.

FIG. 28A-28C demonstrate inhibition of native SARS-CoV-2 infection in vivo. FIG. 28A Reduction of viral load in the lungs of hamsters treated prophylactically with RNA-1 (20 ug in PBS) administered intranasally 1 day prior to intranasal administration of SARS-CoV-2 virus (10² PFU), on the day of infection, and 1 day post-infection, as measured one day later by qPCR for subgenomic RNA encoding SARS-CoV-2 N protein (left; *, p=0.030) or by quantifying viral titers in a plaque assay (right; *, p=0.032). FIG. 28B Reduction of viral load in the lungs of hamsters produced by administering RNA-1 (20 ug) intranasally once a day for two days beginning 1 day after intranasal administration of SARS-CoV-2 virus (103 PFU) and measured one day later by qPCR for subgenomic RNA encoding SARS-CoV-2 N protein (* p=0.01). FIG. 28C Low (left) and high magnification (right) histological H&E-stained images of lungs from B that were treated with the delivery vehicle alone (top) or with vehicle containing RNA-1 (bottom) beginning 1 after infection (left bar, 2.5 mm; right bar, 100 m).

FIGS. 29A and 29B demonstrate profiling the effects of RNA-2 by RNA-seq and TMT mass spectrometry. A549 cells were transfected with RNA-2 or scrambled RNA control. Cell lysates were collected at 48 h, and analyzed by RNA-seq (FIG. 29A) or TMT Mass Spec (FIG. 29B). Differentially expressed genes (DEGs) or proteins are shown in volcano plots (top) and GO Enrichment analysis was performed for the DEGs (bottom) (N=3). Plot (top) and GO Enrichment analysis was performed for the differentially expressed proteins (bottom) (N=3).

FIG. 30 demonstrate subgenomic N transcript for RNA-1 as compared to a vehicle control. Levels are relative to an actin loading control.

### DETAILED DESCRIPTION

The compositions and methods described herein relate, in part, to the discovery of immunomodulatory/immunostimulatory oligonucleotide RNA duplexes that induce interferon (IFN) production. The immunostimulatory oligonucleotide duplexes described herein have the ability to induce robust innate immune responses and inhibit or treat diseases treatable with, or that benefit from increases in, interferons, including but not limited to viral, bacterial, fungal and/or parasitic infections, cancer and autoimmune diseases.

The following describes considerations to permit one of ordinary skill in the art to make and use the subject technology.

Interferons (IFN or IFNs) are a class of pleiotropic cytokines that are produced and released by immune cells as a part of the innate immune response to infections. IFNs have been used as a therapeutic in the treatment of autoimmune diseases (e.g., multiple sclerosis and lupus), many types of cancer, and viral infections. See, e.g., Paolicelli, D., Direnzo, V., & Trojano, M. (2009), Review of interferon beta-1b in the treatment of early and relapsing multiple sclerosis. Biologics: targets & therapy, 3, 369-376; Tamura T, Yanai H, Savitsky D, Taniguchi T., The IRF family transcription factors in immunity and oncogenesis. Annu Rev Immunol. (2008); McNab F, Mayer-Barber K, Sher A, Wack A, O'Garra A., Type I interferons in infectious disease Nat Rev Immunol. (2015), each of which is incorporated herein by reference in its entirety.

Immunomodulatory effects of IFNs are exerted on a wide range of cell types expressing receptors for the interferon polypeptide(s). Downstream effects of interferons allow for the regulation of the immune system by activating signal transducer and activator of transcription (STAT) complexes and other signaling molecules. STATs are a family of transcription factors that regulate the expression of a number of immune system genes. Interferon signaling pathways are known in the art—see e.g., Muller U, et al. Functional role of type I and type II interferons in antiviral defense. Science (1994); Honda et al, Immunity, 25, 349-360 (2006); Marchetti M, et al. Stat-mediated signaling induced by type I and type II interferons (IFNs) is differentially controlled through lipid microdomain association and clathrin-dependent endocytosis of IFN receptors. Mol Biol Cell (2006); Lee and Ashkar, Front. Immunol., 2018; Platanias LC. Mechanisms of type-I- and type-II-interferon-mediated signalling. Nat Rev Immunol. (2005) 5:375-86; each of which are incorporated herein by reference in their entirety.

The induction of interferon (IFN) production plays a critical role in human immune responses by ‘interfering’ with viral replication. Induction of IFN gene expression can lead to increased cellular resistance to infection, including but not limited to viral infection, by activating immune cells, (e.g., natural killer cells and macrophages), and increasing host defenses by upregulating antigen presentation by virtue of increasing the expression of major histocompatibility complex (MHC) antigens. There are a number of types of IFN genes and proteins, which are typically divided among three classes in humans: Type I IFN (IFN-α, IFN-β, IFN-ε, IFN-κ and IFN-ω), Type II IFN (IFN-γ), and Type III IFN. IFNs belonging to all three classes participate in fighting infection and regulating the immune system.

The regulation of IFN expression is complex and tightly controlled by interferon regulatory factors (IRFs). IRFs are a family of transcription factors that are involved in many aspects of the immune response, including development and differentiation of immune cells and regulating responses to pathogens. The functional role and signaling pathways of IRFs are known in the art, see e.g., Jefferries, Front. Immunol., 2019; and Bustamante et al. Clinical immunology, 5^{th} ed. (2019), which are incorporated herein by reference in their entirety. One such IRF, IRF3, is a positive regulator of type I interferon gene induction. IRF3 is an intracellular polypeptide that is activated downstream of the pattern recognition receptor, RIG-I, an intracellular RNA sensor. In particular, IRF3 can directly induce the expression of cytokines, such as IFN-β and in addition to type I IFNs, CXCL10, RANTES, ISG56, IL-12p35, IL-23, and IL-15, whilst inhibiting IL-12β and TGF-β.

The interferon pathways are involved in many diseases, including pathogenic infections caused by viruses, bacteria, fungi and parasites, as well as cancers, and autoimmune diseases. In many instances, an increase in interferon production is part of the natural response to infection, such that treatments that further promote such production can assist in fighting the infection. In other instances, notably some viral infections, including infection with the SARS-CoV-2 coronavirus, among others, the body's interferon response is not activated or is suppressed relative to that seen with other viruses or pathogens, such that a treatment that promotes interferon production can assist in fighting the infection. Therefore, the immunostimulatory oligonucleotide duplexes described herein can be used to prevent, mitigate, and/or treat diseases that benefit from or are treatable with agents that include interferons or that promote interferon production.

### Immunomodulatory Oligonucleotide Duplex Compositions

The immunomodulatory oligonucleotide duplexes disclosed herein are characterized by the 5′-terminal sequence 5′-CUGA-3′ (SEQ ID NO: 1), in complex with its complement, 5′-UCAG-3′, wherein that complement includes a 3′ GG overhang. Thus, the immunostimulatory oligonucleotide duplex comprises the following sequences:

| | | |
|---|---|---|
| Top strand 5′ > 3′ | 5′-CUGAN₁₆-3′ | SEQ ID NO: 41 |
| Bottom strand 3′ > 5′ | 3′-GGGACUN′₁₆-5′ | SEQ ID NO: 2 |

underlined-3′ overhang;
N = any of G, A, C or U or modified versions
thereof;
N′ = complementary bases to N
bold-complementary portions of SEQ ID NO: 1
and SEQ ID NO: 2;

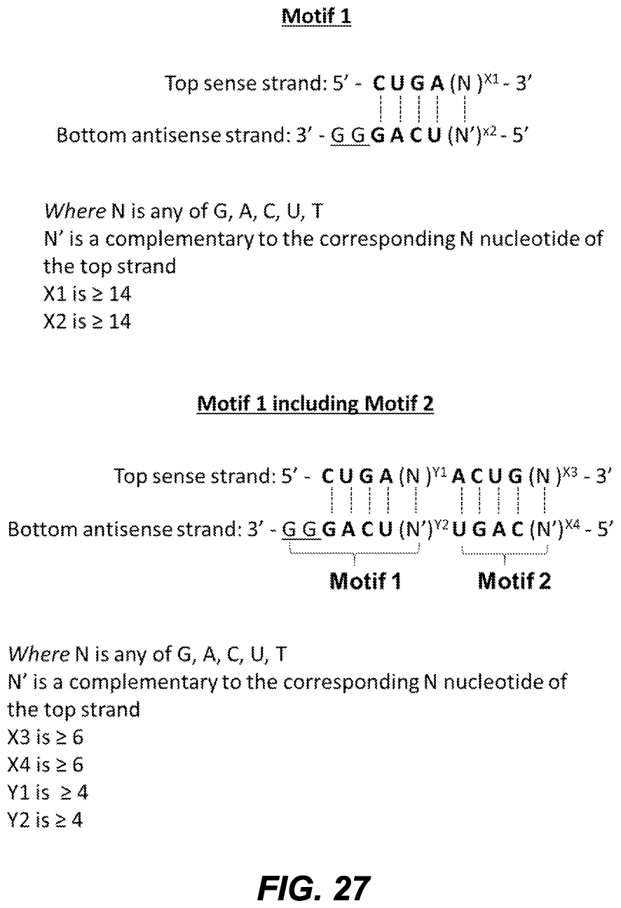

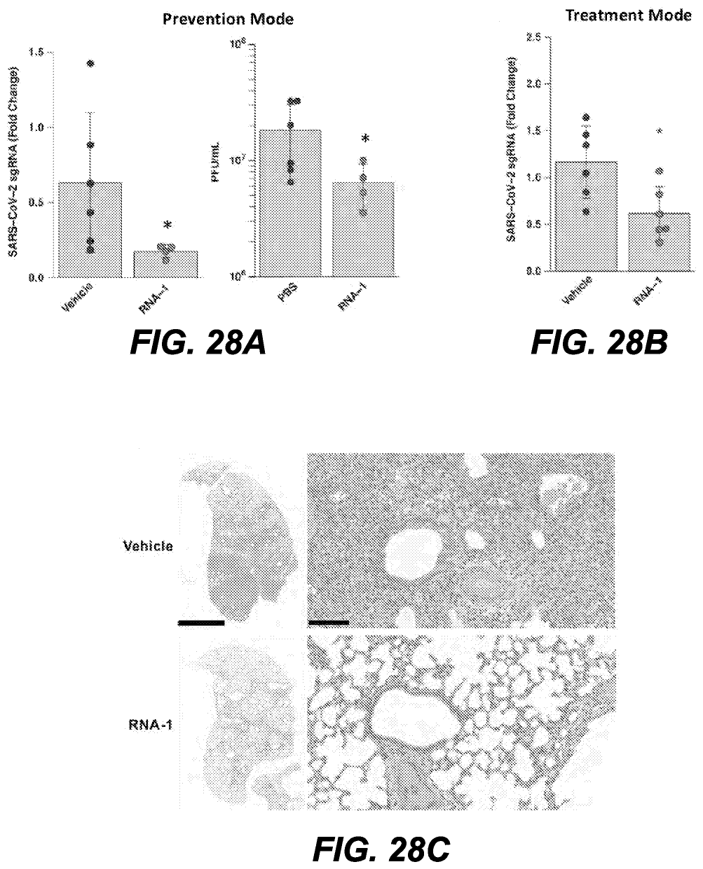

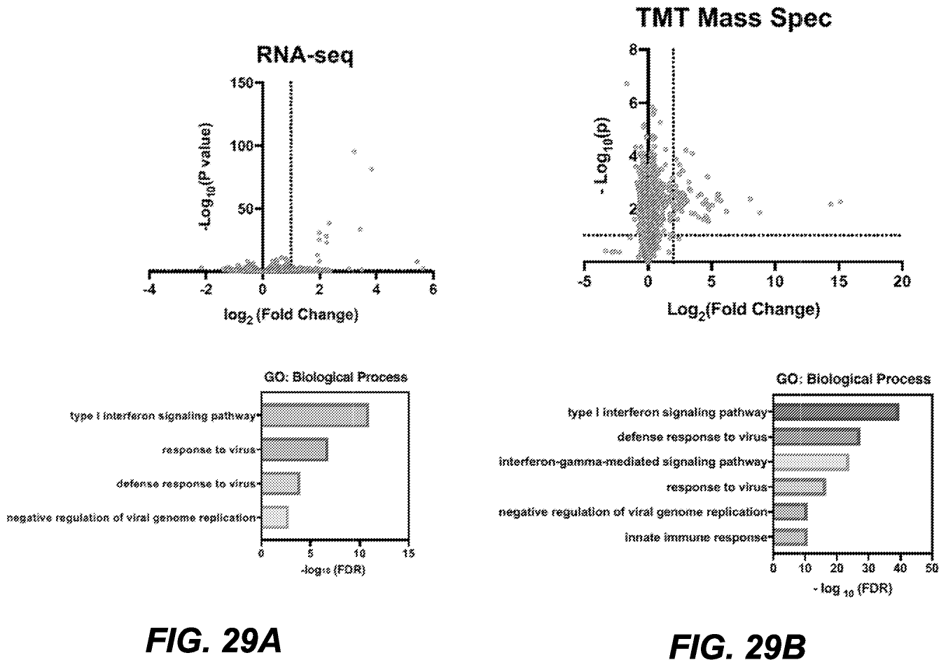

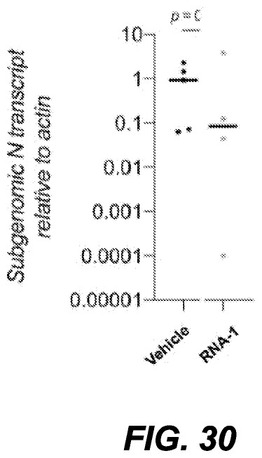

The immunostimulatory oligonucleotide duplexes disclosed herein include duplexed RNA, have a 5′ monophosphate on the 5′-CUGA-3′ (SEQ ID NO: 1), and a minimum duplexed length of 20 nucleotides (see also FIG. 27). The sequence makeup of the duplex 3′ of the 5′-monophosphate-CUGA-3′ (SEQ ID NO: 1) sequence is not critical to the interferon induction. N16 is a minimum. However, N (and the corresponding N′ complementary sequence) can be longer. As discussed elsewhere herein, it is contemplated that the duplex can tolerate some degree of mismatch, but generally, no more than 5 of the N₁₆:N′₁₆ nucleobases should be mismatched. General rules for mismatches, if present, are also discussed elsewhere herein.

In some embodiments of any of the aspects, the immunostimulatory oligonucleotide duplex is at least 20 nucleobases in length. In some embodiments, the immunostimulatory oligonucleotide duplex has a length of 20-300, 20-250, 20-200, 20-150, 20-100, 20-50, 50-300, 50-250, 50-200, 50-150 or 50-100 nucleotides. In some embodiments, the immunostimulatory oligonucleotide duplex has a length of 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100 nucleotides. These lengths are exclusive of the non-duplexed 3′GG overhang on the bottom strand.

In some embodiments, the immunostimulatory oligonucleotide duplexes described herein can be conjugated to an antigen or a biomolecule. In some embodiments, the immunostimulatory oligonucleotide duplexes described herein further comprise a linker. The linker described herein can be used for conjugation of the oligonucleotide sequence to the antigen-coding sequence of the antigen.

### Modifications/Substitutions

It is contemplated that oligonucleotide duplex sequences as described herein can comprise modified nucleotides including modifications to nucleobase and/or sugar-phosphate backbone moieties, as long as the modified nucleotides permit base pairing to the appropriate nucleotide on the opposing strand and as long as such modification(s) permit the resulting duplex molecule to promote interferon production, e.g., as measured using methods known in the art or described herein. Such modifications can alter stability of the duplex, e.g., by reducing susceptibility to enzymatic or chemical degradation, or can modify (increase or decrease) intra- or inter-molecular interactions, including but not limited to base-pairing interactions. RNA oligonucleotide duplex nucleobases include the purine bases adenine (A) and guanine (G), and the pyrimidine bases cytosine (C), and uracil (U) or modified or related forms thereof.

In one embodiment, the duplex sequence comprises one or more modified ribonucleotides in the 5′-monophosphate-CUGA-3′ (SEQ ID NO: 1) sequence or in the 5′-UCAGGG-3′ sequence.

(SEQ IN NO: 41)
5′-monophosphate-CUGAN₁₆
(SEQ ID NO: 2)
3′-GGGACUN′₁₆

In another embodiment, the duplex comprises one or more modified ribonucleotides in the N₁₆ or N′₁₆ sequence or elsewhere in the duplex when the duplex is longer than 20 nucleotides. It is contemplated that modifications that permit, for example, translation of an RNA comprising such modifications would be likely to be tolerated and retain immunostimulatory/interferon-inducing activity in the context of the duplexes described herein. It is contemplated that one or more, two or more, three or more, including all four of the ribonucleotides 5-CUGA-3′ (SEQ IN NO: 1) can be modified in a given duplex molecule. It is further contemplated that one or more, two or more, three or more, four or more, five or more, including all six of the ribonucleotides 5′-UCAGGG-3′ can be modified in a given duplex molecule. It is further contemplated that the N₁₆ or N′₁₆ sequence can include modifications to one or more, two or more, three or more, four or more, five or more, six or more, seven or more, eight or more, nine or more, ten or more, eleven or more, twelve or mote, thirteen or more, fourteen or more, fifteen or more, up to and including all ribonucleotides comprising one or more nucleobase or ribose-phosphate backbone modifications. Similarly, when the N—N′ duplex comprises more than 16 ribonucleotides, any one or any combination of them, up to and including all of them, can include one or more modifications to the nucleobase or ribose-phosphate backbone structure.

Exemplary nucleic acid modifications include, but are not limited to, nucleobase modifications, sugar modifications, inter-sugar linkage modifications, conjugates (e.g., ligands), and combinations thereof. In one embodiment, a modification does not include replacement of a ribose sugar with a deoxyribose sugar as occurs in deoxyribonucleic acid. Nucleic acid modifications are known in the art, see, e.g., US20160367702A1; US20190060458A11; U.S. Pat. Nos. 8,710,200; and 7,423,142, which are incorporated herein by reference in their entireties.

Exemplary modified nucleobases include, but are not limited to, thymine (T), inosine, xanthine, hypoxanthine, nubularine, isoguanisine, tubercidine, and substituted or modified analogs of adenine, guanine, cytosine and uracil, such as 2-aminoadenine, 6-methyl and other alkyl derivatives of adenine and guanine, 2-propyl and other alkyl derivatives of adenine and guanine, 5-halouracil and cytosine, 5-propynyl uracil and cytosine, 6-azo uracil, cytosine and thymine, 5-uracil (pseudouracil), 4-thiouracil, 5-halouracil, 5-(2-aminopropyl)uracil, 5-amino allyl uracil, 8-halo, amino, thiol, thioalkyl, hydroxyl and other 8-substituted adenines and guanines, 5-trifluoromethyl and other 5-substituted uracils and cytosines, 7-methylguanine, 5-substituted pyrimidines, 6-azapyrimidines and N-2, N-6 and 0-6 substituted purines, including 2-aminopropyladenine, 5-propynyluracil and 5-propynylcytosine, dihydrouracil, 3-deaza-5-azacytosine, 2-aminopurine, 5-alkyluracil, 7-alkylguanine, 5-alkyl cytosine, 7-deazaadenine, N6, N6-dimethyladenine, 2,6-diaminopurine, 5-amino-allyl-uracil, N3-methyluracil, substituted 1,2,4-triazoles, 2-pyridinone, 5-nitroindole, 3-nitropyrrole, 5-methoxyuracil, uracil-5-oxyacetic acid, 5-methoxycarbonylmethyluracil, 5-methyl-2-thiouracil, 5-methoxycarbonylmethyl-2-thiouracil, 5-methylaminomethyl-2-thiouracil, 3-(3-amino-3carboxypropyl)uracil, 3-methylcytosine, 5-methylcytosine, N4-acetyl cytosine, 2-thiocytosine, N6-methyladenine, N6-isopentyladenine, 2-methylthio-N6-isopentenyladenine, N-methylguanines, or O-alkylated bases. Further purines and pyrimidines include those disclosed in U.S. Pat. No. 3,687,808, those disclosed in the Concise Encyclopedia of Polymer Science and Engineering, pages 858-859, Kroschwitz, J. I., ed. John Wiley & Sons, 1990, and those disclosed by Englisch et al., Angewandte Chemie, International Edition, 1991, 30, 613.

Exemplary sugar modifications include, but are not limited to, 2′-Fluoro, 3′-Fluoro, 2′-OMe, 3′-OMe, and acyclic nucleotides, e.g., peptide nucleic acids (PNA), unlocked nucleic acids (UNA) or glycol nucleic acid (GNA).

In some embodiments, a nucleic acid modification can include replacement or modification of an inter-sugar linkage. Exemplary inter-sugar linkage modifications include, but are not limited to, phosphotriesters, methylphosphonates, phosphoramidate, phosphorothioates, methylenemethylimino, thiodiester, thionocarbamate, siloxane, N,N′-dimethylhydrazine (—CH2-N(CH3)-N(CH3)-), amide-3 (3′-CH2-C(═O)—N(H)-5′) and amide-4 (3′-CH2-N(H)—C(═O)-5′), hydroxylamino, siloxane (dialkylsiloxxane), carboxamide, carbonate, carboxymethyl, carbamate, carboxylate ester, thioether, ethylene oxide linker, sulfide, sulfonate, sulfonamide, sulfonate ester, thioformacetal (3′-S-CH2-O-5′), formacetal (3′-O-CH2-O-5′), oxime, methyleneimino, methylenecarbonylamino, methylenemethylimino (MMI, 3′-CH2-N(CH3)-O-5′), methylenehydrazo, methylenedimethylhydrazo, methyleneoxymethylimino, ethers (C3′-O—C5′), thioethers (C3′-S—C5′), thioacetamido (C3′-N(H)—C(═O)—CH2-S—C5′, C3′-O—P(O)—O—SS—C5′, C3′-CH2-NH—NH—C5′, 3′-NHP(O)(OCH3)-O-5′ and 3′-NHP(O)(OCH3)-O-5′

In some embodiments, nucleic acid modifications can include peptide nucleic acids (PNA), bridged nucleic acids (BNA), morpholinos, locked nucleic acids (LNA), glycol nucleic acids (GNA), threose nucleic acids (TNA), or other xeno nucleic acids (XNA) described in the art.

In some embodiments, an immunostimulatory oligonucleotide duplex can be in the form of a hairpin intramolecular duplex, or a hairpin-loop intramolecular duplex. In such embodiments, the 5′ and 3′ terminal sequences are self-complementary and provide the 5-monophosphate-CUGAN₁₆ (SEQ ID NO: 41) hybridized to the 5′-N′₁₆UCAGGG-3′ (SEQ ID NO: 2) structure common to the immunostimulatory duplexes disclosed herein.

In another embodiment of any of the aspects, the oligonucleotide duplex described herein comprises a linker. For example, the linker can simply be a nucleic acid backbone linkage e.g., phosphodiester linkage. In addition, the nucleic acid linkers can all be the same, all different, or some are the same and some are different.

In some embodiments of any of the aspects, the linker or spacer can be selected from the group consisting of: photocleavable linkers, hydrolyzable linkers, redox cleavable linkers, phosphate-based cleavable linkers, acid cleavable linkers, ester-based cleavable linkers, peptide-based cleavable linkers, and any combinations thereof. In some embodiments, the cleavable linker can comprise a disulfide bond, a tetrazine-trans-cyclooctene group, a sulfhydryl group, a nitrobenzyl group, a nitoindoline group, a bromo hydroxycoumarin group, a bromo hydroxyquinoline group, a hydroxyphenacyl group, a dimethozybenzoin group, or a combination thereof.

In some embodiments, the immunostimulatory oligonucleotide duplexes described herein are cross-linked such that the complementary strands are covalently joined. Such cross-linking can provide, for example, improved duplex stability, such that the terminal 5′-CUGA′3′ (SEQ ID NO: 1) sequence identified herein is better retained in its active conformation. In some embodiments, the cross-linking moiety can be a chemical functional group. In some embodiments, said chemical functional group is selected from the group consisting of: azide, alkyne, tetrazine, DBCO, thiol, amine, carbonyl, carboxyl group, and any combinations thereof.

In some embodiments, the immunostimulatory oligonucleotide duplexes described herein are cross-linked by a photo-cross linking moiety. Non-limiting examples of photo-crosslinking moieties include, 3-Cyanovinylcarbazole (CNVK) nucleotide; 5-bromo deoxycytosine; 5-iodo deoxycytosine; 5-bromo deoxyurdine; 5-iodo deoxyuridine; and nucleotides comprising an aryl azide (AB-dUMP), benzophenone (BP-dUMP), perfluorinated aryl azide (FAB-dUMP) or diazirine (DB-dUMP).

In some embodiments, the immunostimulatory oligonucleotide duplexes described herein are conjugated to a pharmaceutically acceptable carrier. In other embodiments, the immunostimulatory oligonucleotide duplexes described herein are admixed with a pharmaceutically acceptable carrier.

In some embodiments of any of the aspects, immunostimulatory oligonucleotide duplexes described herein are conjugated to an antigen or antigenic fragment thereof or a sequence encoding an antigen or antigenic fragment thereof.

In some embodiments, an immunostimulatory oligonucleotide duplex as described herein can be fused to or otherwise include a sequence encoding an antigen. Such a composition will include a single-stranded RNA sequence encoding the antigen, fused to or in complex with RNA providing the terminal 5′-monophosphate-CUGA-3′ (SEQ ID NO: 1) and 5′-UCAGGG-3′ duplex/overhang structure shared by immunostimulatory oligonucleotide duplexes as described herein. Introduction of such a composition to a cell can result in both production of antigen to stimulate an adaptive immune response and concomitant stimulation of an interferon response.

### Methods of Preparing Oligonucleotide Duplexes

The immunostimulatory oligonucleotide duplexes described herein can be prepared by synthetic methods known in the art including, but not limited to, chemical synthesis, including but not limited to a nucleoside phosphoramidite approach, or in vitro transcription among others. Methods for chemical synthesis to include modified nucleotides are also known in the art.

In in vitro transcription, polymerases can be used including, but not limited to, bacteriophage polymerase such as T7 polymerase, T3 polymerase and SP6 polymerase, viral polymerases, and E. coli RNA polymerase.

Oligonucleotide strands can be isolated from a sample using RNA extraction and purification methods know in the art. These methods include but are not limited to column purification, ethanol precipitation, phenol-chloroform extraction, or acid guanidinium thiocyanate-phenol chloroform extraction (AGPC). Following isolation of a single stranded oligonucleotide, hybridizing and/or annealing the top and bottom strands can be performed to form the duplex secondary structure.

As used herein, the term “hybridizing”, “hybridize”, “hybridization”, “annealing”, or “anneal” are used interchangeably in reference to the pairing of complementary nucleic acids using any process by which a strand of nucleic acid joins with a complementary strand through base pairing to form a hybridization complex. In other words, the term “hybridization” refers to the process in which two single-stranded polynucleotides bind non-covalently to form a double-stranded polynucleotide. The resulting double-stranded polynucleotide is a “hybrid” or “duplex.” Conditions for forming hybridized or duplexed sequences are known to those of skill in the art, and generally include salt concentration and temperature at or near normal physiological conditions, e.g., intracellular conditions. Generally, hybridization to form duplexes as described herein can be performed with each strand present in substantially equimolar concentrations.

Following synthesis, hybridization and, optionally, removal of non-duplexed strands, the immunostimulatory oligonucleotide duplexes can be characterized by any method known in the art, e.g., liquid chromatography, mass spectrometry, next generation sequencing, polymerase chain reaction (PCR), gel electrophoresis, or any other method of identifying nucleoside sequences, secondary structures, chemical composition, expression, thermodynamics, binding, or function.

For further characterization of the immunostimulatory oligonucleotide duplexes described herein, the 5′-monophosphate can be detected, for example, by a splinted ligation assay. See e.g., Shoenberg et al, Nat Chem Biol 3(9) (2007) and Celesnik H et al. Initiation of RNA decay in Escherichia coli by 5′ pyrophosphate removal. Mol Cell. 2007; 27:79-90, which are incorporated herein by reference in their entireties. By carefully optimizing reaction conditions and comparing ligated with unligated RNA this assay yields quantitative data of the amount of RNA with a 5′ monophosphate end.

In order to improve the stability or produce any of the oligonucleotide modifications described above, immunostimulatory oligonucleotide duplexes as described herein, may be chemically modified in a suitable manner. As noted above, modifications can be made in order to meet the requirements of stability of the oligonucleotide duplexes toward extra- and intracellular enzymes and ability to penetrate through the cell membrane for human therapeutic applications. See, e.g., Uhlmann, E.; Peyman, A. Chem. Rev. 1990, 90, 544; Milligan, J. F.; Matteucci, M. D.; Martin, J. C. J. Med. Chem. 1993, 36, 1923; Crooke, S. T.; Lebleu, B., Eds. 1993, Antisense research and applications; CRC Press: Boca Raton, Fla.; and Thuong, N. T.; Helene, C. Angew. Chim. Int. Ed. 1993, 32, 666. Chemical modifications to nucleic acids may include introduction of heterocyclic bases, phosphate backbone modifications, sugar moiety modifications, and attachment of conjugated groups. See Beaucage, S. L.; Iyer, R. P. Tetrahedron 1993, 49, 1925; Beaucage, S. L.; Iyer, R. P. Tetrahedron 1993, 49, 6123; Manoharan, M. Antisense Technology, 2001, S. T. Crooke, ed. (Marcel Dekker, New York); and Manohran, M. Antisense & Nucleic acid Development 2002, 12, 103, Schweitzer, B. A.; Kool, E. T. J. Org. Chem. 1994, 59, 7238; Schweitzer, B. A.; Kool, E. T. J. Am. Chem. Soc. 1995, 117, 1863; Moran, S. Ren, R. X.-F. Rumney, S.; Kool, E. T. J. Am. Chem. Soc. 1997, 119, 2056; Guckian, K. M.; Kool, E. T. Angew. Chem. Int. Ed. Engl. 1997, 36, 2825; and Mattray, T. J.; Kool, E. T. J. Am. Chem. Soc. 1998, 120, 6191. For additional information see Fire, A.; Xu, S.; Montgomery, M. K.; Kostas, S. A.; Driver, S. E.; Mello, C. C. Nature, 1998, 391, 806; Elbashir, S. M.; Harborth, J.; Lendeckel, W.; Yalcin, A.; Weber, K.; Tuschl, T. Nature, 2001, 411, 494; McManus, M. T. Sharp, P. A. Nature Reviews Genetics, 2002, 3, 737; Hannon, G. J. Nature, 2002, 418, 244; and Roychowdhury, A.; IIIangkoon, H.; Hendrickson, C. L.; Benner, S. A. Org. Lett. 2004, 6, 489, which are incorporated herein by reference in their entireties.

For some therapeutic purposes, immunostimulatory oligonucleotide duplexes described herein should have a degree of stability in serum to allow distribution and cellular uptake. The prolonged maintenance of therapeutic levels of the oligonucleotides in serum will have a significant effect on the distribution and cellular uptake and unlike conjugate groups that target specific cellular receptors, the increased serum stability will affect all cells.

Chemical modifications can also include the addition of ligands, linkers, and antigens. For example, the ligand can improve stability, hybridization thermodynamics with a target nucleic acid, targeting to a particular tissue or cell-type, or cell permeability, e.g., by an endocytosis-dependent or independent mechanism. Oligonucleotides bearing peptide (e.g. antigen) conjugates can be prepared using procedures known in the art. See Trufert et al., Tetrahedron 1996, 52, 3005; and Manoharan, “Oligonucleotide Conjugates in Antisense Technology,” in Antisense Drug Technology, ed. S. T. Crooke, Marcel Dekker, Inc., 2001, each of which is hereby incorporated by reference.

### Pharmaceutical Compositions

The methods and oligonucleotide duplex compositions described herein can further comprise formulating the immunostimulatory oligonucleotide duplexes described herein with a pharmaceutically acceptable carrier.

In some embodiments of any of the aspects, the method further comprises formulating the immunostimulatory oligonucleotide duplexes with a pharmaceutically acceptable carrier and an antigen or a nucleic acid sequence encoding an antigen. Such formulations exploit the immunostimulatory duplexes as described herein to provide an adjuvant effect, e.g., when the formulation is administered as or in conjunction with a vaccine. In some embodiments of any of the aspects, the method further comprises formulating the immunostimulatory oligonucleotide duplexes with a pharmaceutically acceptable carrier, an antigen or a nucleic acid sequence encoding an antigen, and a separate adjuvant.

For clinical use of the methods and compositions described herein, administration of the immunostimulatory oligonucleotide duplexes described herein can include formulation into pharmaceutical compositions or pharmaceutical formulations for parenteral administration, e.g., intravenous; mucosal, e.g., intranasal; ocular, or other mode of administration. In some embodiments, the immunostimulatory oligonucleotide duplex described herein can be administered along with any pharmaceutically acceptable carrier compound, material, or composition which results in an effective treatment in the subject. Thus, a pharmaceutical formulation for use in the methods described herein can contain the immunostimulatory oligonucleotide duplex described herein in combination with one or more pharmaceutically acceptable ingredients. The phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” as used herein means a pharmaceutically acceptable material, composition or vehicle, such as a liquid or solid filler, diluent, excipient, solvent, media, encapsulating material, manufacturing aid (e.g., lubricant, talc magnesium, calcium or zinc stearate, or steric acid), or solvent encapsulating material, involved in maintaining the stability, solubility, or activity of, an immunostimulatory oligonucleotide duplex as described herein. Each carrier must be “acceptable” in the sense of being compatible with the other ingredients of the formulation and not injurious to the patient. The terms “excipient,” “carrier,” “pharmaceutically acceptable carrier” or the like are used interchangeably herein.

The immunostimulatory oligonucleotide duplexes described herein can be formulated for administration of the compound to a subject in solid, liquid or gel form, including those adapted for the following: (1) parenteral administration, for example, by subcutaneous, intramuscular, intravenous or epidural injection as, for example, a sterile solution or suspension, or sustained-release formulation; (2) transdermally; (3) transmucosally; (4) via bronchoalveolar lavage.

In some embodiments, the compositions described herein comprise a particle or polymer-based vehicle. Exemplary particle or polymer-based vehicles include, but are not limited to, nanoparticles, microparticles, polymer microspheres, or polymer-drug conjugates.

In one embodiment of any of the aspects, the compositions described herein further comprise a lipid vehicle. Exemplary lipid vehicles include, but are not limited to, liposomes, phospholipids, micelles, lipid emulsions, and lipid-drug complexes.

Formulations can be adapted for delivery to the airway, e.g., to address respiratory infection. Such formulations can be adapted for delivery as an aerosol, e.g., for inhalation. In some embodiments, the compositions described herein are formulated for aerosol administration, nebulizer administration, or tracheal lavage administration. In some embodiments, the composition is formulated for intravenous, intramuscular, intraperitoneal, subcutaneous, or intrathecal administration.

For use as aerosols, the compositions described herein can be prepared in a solution or suspension and may be packaged in a pressurized aerosol container together with suitable propellants, for example, hydrocarbon propellants like propane, butane, or isobutane with conventional excipients.

The oligonucleotide duplex compositions described herein can also be administered in a non-pressurized form such as in a nebulizer or atomizer that reduces a liquid to a fine spray. Preferably, by such nebulization small liquid droplets of uniform size are produced from a larger body of liquid in a controlled manner. Nebulization can be achieved by any suitable means therefor, including by using many nebulizers known and marketed today. For example, an AEROMIST™ pneumatic nebulizer available from Inhalation Plastic, Inc. of Niles, Ill.

When the active ingredients are adapted to be administered, either together or individually, via nebulizer(s) they can be in the form of a nebulized aqueous suspension or solution, with or without a suitable pH or tonicity adjustment, either as a unit dose or multi-dose device.

Furthermore, any suitable gas can be used to apply pressure during the nebulization, with preferred gases to date being those which are chemically inert. Exemplary gases including, but not limited to nitrogen, argon, or helium can be used to advantage.

In some embodiments, the compositions described herein can also be administered directly to the airways in the form of a dry powder. Thus, the immunostimulatory oligonucleotide duplexes can be administered via an inhaler. Exemplary inhalers include metered dose inhalers and dry powdered inhalers.

A metered dose inhaler or “MDI” is a pressure resistant canister or container filled with a product such as a pharmaceutical composition dissolved in a liquefied propellant or micronized particles suspended in a liquefied propellant. The propellants which can be used include chlorofluorocarbons, hydrocarbons or hydrofluoroalkanes. Commonly used propellants are P134a (tetrafluoroethane) and P227 (heptafluoropropane) each of which may be used alone or in combination. They are optionally used in combination with one or more other propellants and/or one or more surfactants and/or one or more other excipients, for example ethanol, a lubricant, an anti-oxidant and/or a stabilizing agent.

A dry powder inhaler (i.e., Turbuhaler™ (Astra AB)) is a system operable with a source of pressurized air to produce dry powder particles of a pharmaceutical composition that is compacted into a very small volume.

Dry powder aerosols for inhalation therapy are generally produced with mean diameters primarily in the range of <5 μm. As the diameter of particles exceeds 3 μm, there is increasingly less phagocytosis by macrophages. However, increasing the particle size also has been found to minimize the probability of particles (possessing standard mass density) entering the airways and acini due to excessive deposition in the oropharyngeal or nasal regions.

Suitable powder compositions include, by way of illustration, powdered preparations including the immunostimulatory oligonucleotide duplexes described herein. These can be intermixed with lactose, or other inert powders acceptable for intrabronchial administration. The powder compositions can be administered via an aerosol dispenser or encased in a breakable capsule which may be inserted by the patient or clinician into a device that punctures the capsule and blows the powder out in a steady stream suitable for inhalation. The compositions can include propellants, surfactants, and co-solvents and may be filled into conventional aerosol containers that are closed by a suitable metering valve.

Aerosols for the delivery to the respiratory tract are described, for example, by Adjei, A. and Garren, J. Pharm. Res., 1: 565-569 (1990); Zanen, P. and Lamm, J.-W. J. Int. J. Pharm., 114: 111-115 (1995); Gonda, I. “Aerosols for delivery of therapeutic and diagnostic agents to the respiratory tract,” in Critical Reviews in Therapeutic Drug Carrier Systems, 6:273-313 (1990); Anderson et al., Am. Rev. Respir. Dis., 140: 1317-1324 (1989)) and have potential for the systemic delivery of peptides and proteins as well (Patton and Platz, Advanced Drug Delivery Reviews, 8:179-196 (1992)); Timsina et. al., Int. J. Pharm., 101: 1-13 (1995); and Tansey, I. P., Spray Technol. Market, 4:26-29 (1994); French, D. L., Edwards, D. A. and Niven, R. W., Aerosol Sci., 27: 769-783 (1996); Visser, J., Powder Technology 58: 1-10 (1989)); Rudt, S. and R. H. Muller, J. Controlled Release, 22: 263-272 (1992); Tabata, Y, and Y. Ikada, Biomed. Mater. Res., 22: 837-858 (1988); Wall, D. A., Drug Delivery, 2: 10 1-20 1995); Patton, J. and Platz, R., Adv. Drug Del. Rev., 8: 179-196 (1992); Bryon, P., Adv. Drug. Del. Rev., 5: 107-132 (1990); Patton, J. S., et al., Controlled Release, 28: 15 79-85 (1994); Damms, B. and Bains, W., Nature Biotechnology (1996); Niven, R. W., et al., Pharm. Res., 12(9); 1343-1349 (1995); and Kobayashi, S., et al., Pharm. Res., 13(1): 80-83 (1996), the contents of each of which are incorporated herein by reference in their entirety.

In addition to chemical modification of the immunostimulatory oligonucleotide duplexes described herein, efforts aimed at improving the transmembrane delivery of nucleic acids and oligonucleotides have utilized protein carriers, antibody carriers, liposomal delivery systems, electroporation, direct injection, cell fusion, viral vectors, and calcium phosphate-mediated transformation. U.S. Pat. Nos. 7,423,142 B2, 7,786,290 B2, 8,598,139 B2, 8,808,747 B2, 10,125,369 B2, 10,130,649 B2, and U.S. patent publication 2018/0369419 A1, each of which is incorporated herein by reference, describe formulations for delivery of mRNA, siRNA, and dsRNA compositions to skin, blood, liver, and other target tissues or organs. As but one example, U.S. Pat. No. 8,598,139 B2 provides several examples of nucleic acid-lipid particle formulations for delivery; see, e.g., columns 42-48. Where the interferon-inducing molecules disclosed herein also have duplex characteristics, it is specifically contemplated that formulations for delivery of siRNA compositions to such tissues can be used to deliver the duplexes disclosed herein.

In some embodiments the immunostimulatory oligonucleotide duplexes as described herein are formulated in a composition comprising micelles, amphiphilic carriers, polymers, cyclodextrins, liposomes, and encapsulation devices.

Microemulsification technology can improve bioavailability of some lipophilic (water insoluble) pharmaceutical agents. Examples include Trimetrine (Dordunoo, S. K., et al., Drug Development and Industrial Pharmacy, 17(12), 1685-1713, 1991 and REV 5901 (Sheen, P. C., et al., J Pharm Sci 80(7), 712-714, 1991). Among other things, microemulsification provides enhanced bioavailability by preferentially directing absorption to the lymphatic system instead of the circulatory system, which thereby bypasses the liver, and prevents destruction of the compounds in the hepatobiliary circulation.

The immunostimulatory oligonucleotide duplexes as described herein can be formulated with an amphiphilic carrier. Amphiphilic carriers are saturated and monounsaturated polyethyleneglycolyzed fatty acid glycerides, such as those obtained from fully or partially hydrogenated various vegetable oils. Such oils may advantageously consist of tri-. di- and mono-fatty acid glycerides and di- and mono-polyethyleneglycol esters of the corresponding fatty acids, with a particularly preferred fatty acid composition including capric acid 4-10, capric acid 3-9, lauric acid 40-50, myristic acid 14-24, palmitic acid 4-14 and stearic acid 5-15%. Another useful class of amphiphilic carriers includes partially esterified sorbitan and/or sorbitol, with saturated or mono-unsaturated fatty acids (SPAN-series) or corresponding ethoxylated analogs (TWEEN-series).

Commercially available amphiphilic carriers are particularly contemplated, including Gelucire-series, Labrafil, Labrasol, or Lauroglycol (all manufactured and distributed by Gattefosse Corporation, Saint Priest, France), PEG-mono-oleate, PEG-di-oleate, PEG-mono-laurate and di-laurate, Lecithin, Polysorbate 80, etc. (produced and distributed by a number of companies in USA and worldwide).

The immunostimulatory oligonucleotide duplexes as described herein can be formulated with hydrophilic polymers. Hydrophilic polymers are water-soluble, can be covalently attached to a vesicle-forming lipid, and which are tolerated in vivo without toxic effects (i.e., are biocompatible). Suitable polymers include polyethylene glycol (PEG), polylactic (also termed polylactide), polyglycolic acid (also termed polyglycolide), a polylactic-polyglycolic acid copolymer, and polyvinyl alcohol. Other hydrophilic polymers which may be suitable include polyvinylpyrrolidone, polymethoxazoline, polyethyloxazoline, polyhydroxypropyl methacrylamide, polymethacrylamide, polydimethylacrylamide, and derivatized celluloses such as hydroxymethylcellulose or hydroxyethylcellulose.

In certain embodiments, a pharmaceutical composition as described herein comprises a biocompatible polymer selected from the group consisting of polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, or copolymers thereof.

In certain embodiments, a pharmaceutical composition described herein is formulated as a liposome. Liposomes can be prepared by any of a variety of techniques that are known in the art. See, e.g., U.S. Pat. No. 4,235,871; Published PCT applications WO 96/14057; New RRC, Liposomes: A practical approach, IRL Press, Oxford (1990), pages 33-104; Lasic DD, Liposomes from physics to applications, Elsevier Science Publishers BV, Amsterdam, 1993.

In some embodiments of any of the aspects, immunostimulatory oligonucleotide duplexes as described herein can be conjugated to an antigen or antigenic fragment thereof and formulated as a vaccine composition. Therapeutic formulations of the immunostimulatory oligonucleotide duplexes as described herein can be prepared for storage by mixing the immunostimulatory oligonucleotide duplex having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

Vaccine or other pharmaceutical compositions comprising an immunostimulatory oligonucleotide duplex composition as described herein can contain a pharmaceutically acceptable salt, typically, e.g., sodium chloride, and preferably at about physiological concentrations. The formulations of vaccine or other pharmaceutical compositions described herein can contain a pharmaceutically acceptable preservative. In some embodiments, the preservative concentration ranges from 0.1 to 2.0%, typically v/v. Suitable preservatives include those known in the pharmaceutical arts. Benzyl alcohol, phenol, m-cresol, methylparaben, and propylparaben are examples of preservatives. The formulations of vaccine or other pharmaceutical compositions described herein can include a pharmaceutically acceptable surfactant at a concentration of 0.005 to 0.02%.

Therapeutic pharmaceutical compositions described herein can also contain more than one active compound as necessary for the particular indication being treated, preferably those with complementary activities that do not adversely affect each other.

In some embodiments in which the duplexes are formulated for use in or with a vaccine, the vaccine composition can be formulated with the duplex as an adjuvant. In other embodiments the vaccine composition can be formulated with the immunostimulatory oligonucleotide duplex and an additional adjuvant, e.g., as known in the art.

As used herein in the context of immunization, immune response and vaccination, the term “adjuvant” refers to any substance than when used in combination with a specific antigen produces a more robust immune response than the antigen alone. When incorporated into a vaccine formulation, an adjuvant acts generally to accelerate, prolong, or enhance the quality of specific immune responses to the vaccine antigen(s).

Adjuvants typically promote the accumulation and/or activation of accessory cells or factors to enhance antigen-specific immune responses and thereby enhance the efficacy of vaccines, i.e., antigen-containing or encoding compositions used to induce protective immunity against the antigen.

Adjuvants, in general, include adjuvants that create a depot effect, immune-stimulating adjuvants, and adjuvants that create a depot effect and stimulate the immune system. An adjuvant that creates a depot effect is an adjuvant that causes the antigen to be slowly released in the body, thus prolonging the exposure of immune cells to the antigen. This class of adjuvants includes but is not limited to alum (e.g., aluminum hydroxide, aluminum phosphate); emulsion-based formulations including mineral oil, non-mineral oil, water-in-oil or oil-in-water-in oil emulsion, oil-in-water emulsions such as Seppic ISA series of Montanide adjuvants (e.g., Montanide ISA 720; AirLiquide, Paris, France); MF-59 (a squalene-in-water emulsion stabilized with Span 85 and Tween 80; Chiron Corporation, Emeryville, Calif.); and PROVAX™ (an oil-in-water emulsion containing a stabilizing detergent and a micelle-forming agent; IDEC Pharmaceuticals Corporation, San Diego, Calif.).

An immune-stimulating adjuvant is an adjuvant that causes activation of a cell of the immune system. It may, for instance, cause an immune cell to produce and secrete cytokines and interferons. This class of adjuvants includes but is not limited to saponins purified from the bark of the Q. saponaria tree, such as QS21 (a glycolipid that elutes in the 21st peak with HPLC fractionation; Aquila Biopharmaceuticals, Inc., Worcester, Mass.); poly[di(carboxylatophenoxy)phosphazene (PCPP polymer; Virus Research Institute, USA); derivatives of lipopolysaccharides such as monophosphoryl lipid A (MPL; Ribi ImmunoChem Research, Inc., Hamilton, Mont.), muramyl dipeptide (MDP; Ribi) and threonyl-muramyl dipeptide (t-MDP; Ribi); OM-174 (a glucosamine disaccharide related to lipid A; OM Pharma SA, Meyrin, Switzerland); and Leishmania elongation factor (a purified Leishmania protein; Corixa Corporation, Seattle, Wash.). This class of adjuvants also includes CpG DNA.

Adjuvants that create a depot effect and stimulate the immune system are those compounds which have both of the above-identified functions. This class of adjuvants includes but is not limited to ISCOMS (immunostimulating complexes which contain mixed saponins, lipids and form virus-sized particles with pores that can hold antigen; CSL, Melbourne, Australia); SB-AS2 (SmithKline Beecham adjuvant system #2 which is an oil-in-water emulsion containing MPL and QS21: SmithKline Beecham Biologicals [SBB], Rixensart, Belgium); SB-AS4 (SmithKline Beecham adjuvant system #4 which contains alum and MPL; SBB, Belgium); non-ionic block copolymers that form micelles such as CRL 1005 (these contain a linear chain of hydrophobic polyoxypropylene flanked by chains of polyoxyethylene; Vaxcel, Inc., Norcross, Ga.); and Syntex Adjuvant Formulation (SAF, an oil-in-water emulsion containing Tween 80 and a nonionic block copolymer; Syntex Chemicals, Inc., Boulder, Colo.).

The active ingredients of the pharmaceutical compositions described herein can also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions. Such techniques are disclosed in Remington's Pharmaceutical Sciences 16th edition, Osol, A. Ed. (1980).

In some embodiments, sustained-release preparations can be used. Suitable examples of sustained-release preparations include semipermeable matrices of solid hydrophobic polymers containing an antigen or fragment thereof described herein in which the matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated, the antigen or fragment thereof can remain in the body for a long time, denature, or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S— bond formation through thio-disulfide interchange, stabilization can be achieved by modifying sulfhydryl residues, lyophilizing from acidic solutions, controlling moisture content, using appropriate additives, and developing specific polymer matrix compositions.

### Immunostimulatory Activity

The immunostimulatory oligonucleotide duplexes, pharmaceutical compositions, and vaccine compositions described herein can be administered to a subject in need of immunostimulation, and particularly a subject in need of or that would likely to benefit from induction of interferon production. In various embodiments, the interferon-inducing activity is therapeutic on its own, in combination with one or more anti-infectives (e.g., antiviral, antibacterial, antifungal or anti-parasitic), in combination with one or more anti-cancer agents, or in combination with one or more therapeutics for autoimmune disease.

Immunostimulatory activity can be determined, for example, by detecting and measuring the levels of cytokine and interferon production in a biological sample (e.g, serum).

Methods for detecting, measuring, and determining the levels of IFN in a biological sample are known in the art. IFN polypeptide levels can be detected, for example, via immunoassay. ThermoFisher Scientific sells an ELISA-based kit for measuring human interferon gamma levels—see Catalog #29-8319-65. IFN gene expression can also be detected. Methods of measuring gene expression are known in the art, e.g., PCR, microarrays, and immunodetection methods, such as Western blotting and immunocytochemistry, among others. For example, Quantitative reverse transcription polymerase chain reaction (qPCR) analysis can be performed using kits and arrays commercially available from, e.g., Applied Biosystems™—see Applied Biosystems® TaqMan® Array Human Interferon Pathway, catalog #4414154. See also, de Veer M J et al. Functional classification of interferon-stimulated genes identified using microarrays. J Leukoc Biol. (2001) 69:912-20, which are incorporated herein by reference in their entireties.

Antibodies specific for a class of interferon polypeptides (e.g., IFN-γ) are known in the art and can be used in immunohistochemistry, immunofluorescence, and Western Blotting, e.g., commercially available from Abcam™.

Interferon levels and activity can also be determined using a reporter assay or a bioassay. For example, reporter assays for the detection of bioactive type I interferons are available from InvovGen® by monitoring the activation of the ISGF3 pathway. See, e.g., Rees et al. J Immunol Methods, (2018).

Viral infection assays can also be used to determine the effect of the immunostimulatory oligonucleotide duplexes on viral protection. For example, IFN activity can be measured by the level of protection of a cell line against cell death after infection with a virus as compared with a relevant control. See, e.g., Barber et al. Host defense, viruses and apoptosis. Cell Death Differ 8, 113-126, doi: 10.1038/sj.cdd.4400823 (2001); and Liu, S. et al. Science 347, (2015), and which are incorporated herein by reference in its entirety.

In addition, relevant animal models and human in vitro engineered platforms can also be used to detect interferon production directly or indirectly. Any model known in the art can be used. See, e.g, Si, L. et al. Human organs-on-chips as tools for repurposing approved drugs as potential influenza and COVID19 therapeutics in viral pandemics. bioRxiv, doi:10.1101/2020.04.13.039917 (2020); Van den Broek M F, Muller U, Huang S, Zinkernagel R M, Aguet M. Immune defence in mice lacking type I and/or type II interferon receptors. Immunol Rev. (1995).

Providing protection against the relevant pathogen includes stimulating the immune system such that later exposure to a microorganism, antigen, or antigen fragment thereof (e.g., an antigen on or in a live pathogen) triggers a more effective immune response than if the subject was naive to the antigen. Protection can include faster clearance of the pathogen, reduced severity and/or time of symptoms, and/or lack of development of disease or symptoms. As compared with an equivalent untreated control, such reduction is by at least 5%, 10%, 20%, 40%, 50%, 60%, 80%, 90%, 95%, 99% or more as measured by any standard technique.

### Methods of Treatment

The immunostimulatory oligonucleotides described herein can be used for treating IFN-associated diseases, including infection by a wide range of viral, bacterial, fungal, and parasitic pathogens, as well as cancer, and autoimmune diseases, in addition to inhibiting influenza virus infection.

A disease or medical condition is considered to be associated with interferons if administration or induction of interferon production treats the disease or condition. Some diseases or disorders involve interferon induction as part of the healing or recovery process, while in others, the pathology is characterized by deficient, low or nonexistent production of interferons, e.g, IFN, Type I IFN, IFN-α, IFN-β, IFN-ε, IFN-κ and IFN-ω, Type II IFN (IFN-γ), and Type III IFN.

Described herein is a method of treating an infection in a subject in need thereof, the method comprising administering to the subject an immunostimulatory oligonucleotide duplex described herein.

In one embodiment of this or any of the aspects, the oligonucleotide duplex is sufficient to induce interferon (IFN) production in a cell contacted with the duplex. In another embodiment, administering the oligonucleotide duplex to a subject in need thereof is sufficient to increase the levels or activity of IFN. In another embodiment, administering the oligonucleotide duplex to a subject in need thereof is sufficient to increase an immune response in the subject. In another embodiment, the immune response is an anti-viral response.

Without limitations, the immunostimulatory oligonucleotide duplexes described herein can be used to treat a microbial infection. Non-limiting examples of microbes that can cause a microbial infection include viruses, bacteria, fungi and parasites.

In another embodiment, the microbial infection is chronic. In one embodiment, the microbial infection is acute. An acute infection is a short term infection, persisting less than 2 weeks, while a chronic infection is long term, and persists longer than two weeks. The method for treating an acute infection can be the same method used to treat a chronic infection. In contrast, a different method can be used to treat an acute and chronic infection.

In some embodiments of any of the aspects, the microbial infection is a systemic infection. As described herein, “systemic infection” refers to an infection that has spread throughout the body, for example, an infection that is present in the blood. Non-limiting examples of systemic infections include bacterial sepsis and endotoxin shock.

In some embodiments, the microbial infection is caused by a bacterium. Non-limiting examples of bacterial infections that can be treated or prevented by administering an immunostimulatory oligonucleotide duplex described herein includes but is not limited to Aeromonas infection, African tick bite fever, American tick bite fever (Rickettsia parkeri infection), Arcanobacterium haemolyticum infection, Bacillary angiomatosis, Bejel (endemic syphilis), Blastomycosis-like pyoderma (pyoderma vegetans), Blistering distal dactylitis, Botryomycosis, Briii-Zinsser disease, Brucellosis (Bang's disease, Malta fever, undulant fever), Bubonic plague, Bullous impetigo, Cat scratch disease (cat scratch fever, English-Wear infection, inoculation lymphoreticulosis, subacute regional lymphadenitis), Cellulitis, Chancre, Chancroid (soft chancre, ulcus molle), Chlamydia infection, Chronic lymphangitis, Chronic recurrent erysipelas, Chronic undermining burrowing ulcers (Meleney gangrene), Chromobacteriosis infection, Condylomata lata, Cutaneous actinomycosis, Cutaneous anthrax infection, Cutaneous C. diphtheriae infection (Barcoo rot, diphtheric desert sore, septic sore, Veldt sore), Cutaneous group B streptococcal infection, Cutaneous Pasteurella hemolytica infection, Cutaneous Streptococcus iniae infection, Dermatitis gangrenosa (gangrene of the skin), Ecthyma, Ecthyma gangrenosum, Ehrlichiosis ewingii infection, Elephantiasis nostras, Endemic typhus (murine typhus), Epidemic typhus (epidemic louse-borne typhus), Erysipelas (ignis sacer, Saint Anthony's fire), Erysipeloid of Rosenbach, Erythema marginatum, Erythrasma, External otitis (otitis externa, swimmer's ear), Felon, Flea-borne spotted fever, Flinders Island spotted fever, Flying squirrel typhus, Folliculitis, Fournier gangrene (Fournier gangrene of the penis or scrotum), Furunculosis (boil), Gas gangrene (Clostridial myonecrosis, myonecrosis), Glanders (Equinia, farcy, malleus), Gonococcemia (arthritis-dermatosis syndrome, disseminated gonococcal infection), Gonorrhea (clap) Gram-negative folliculitis, Gram-negative toe web infection, Granuloma inguinale (Donovanosis, granuloma genitoinguinale, granuloma inguinale tropicum, granuloma venereum, granuloma venereum genitoinguinale, lupoid form of groin ulceration, serpiginous ulceration of the groin, ulcerating granuloma of the pudendum, ulcerating sclerosing granuloma), Green nail syndrome, Group JK Corynebacterium sepsis, Haemophilus influenzae cellulitis, Helicobacter cellulitis, Hospital furunculosis, Hot tub folliculitis (Pseudomonas aeruginosa folliculitis), Human granulocytotropic anaplasmosis, Human monocytotropic ehrlichiosis, Impetigo contagiosa, Japanese spotted fever, Leptospirosis (Fort Bragg fever, pretibial fever, Weil's disease), Listeriosis, Ludwig's angina, Lupoid sycosis, Lyme disease (Afzelius' disease, Lyme borreliosis), Lymphogranuloma venereum (climatic bubo, Durand-Nicolas-Favre disease, lymphogranuloma inguinale, poradenitis inguinale, strumous bubo), Malakoplakia (malacoplakia), Mediterranean spotted fever (Boutonneuse fever), Melioidosis (Whitmore's disease), Meningococcemia, Missouri Lyme disease, Mycoplasma infection, Necrotizing fasciitis (flesh-eating bacteria syndrome), Neonatal toxic shock-like exanthematous disease, Nocardiosis, Noma neonatorum, North Asian tick typhus, Ophthalmia neonatorum, Oroya fever (Carrion's disease), Pasteurellosis, Perianal cellulitis (perineal dermatitis, streptococcal perianal disease), Periapical abscess, Pinta, Pitted keratolysis (keratolysis plantare sulcatum, keratoma plantare sulcatum, ringed keratolysis), Plague, Primary gonococcal dermatitis, Pseudomonal pyoderma, Pseudomonas hot-foot syndrome, Pyogenic paronychia, Pyomyositis, Q fever, Queensland tick typhus, Rat-bite fever, Recurrent toxin-mediated perineal erythema, Rhinoscleroma, Rickettsia aeschlimannii infection, Rickettsialpox, Rocky Mountain spotted fever, Saber shin (anterior tibial bowing), Saddle nose, Salmonellosis, Scarlet fever, Scrub typhus (Tsutsugamushi fever), Shigellosis, Staphylococcal scalded skin syndrome (pemphigus neonatorum, Ritter's disease), Streptococcal intertrigo, Superficial pustular folliculitis (impetigo of Bockhart, superficial folliculitis), Sycosis vulgaris (barber's itch, sycosis barbae), Syphilid, Syphilis (lues) Tick-borne lymphadenopathy, Toxic shock syndrome (streptococcal toxic shock syndrome, streptococcal toxic shock-like syndrome, toxic streptococcal syndrome), Trench fever (five-day fever, quintan fever, urban trench fever), Tropical ulcer (Aden ulcer, jungle rot, Malabar ulcer, tropical phagedena), Tularemia (deer fly fever, Ohara's disease, Pahvant Valley plague, rabbit fever), Verruga peruana, Vibrio vulnificus infection, Yaws (bouba, frambOsie, parangi, pian), Aquarium granuloma (fish-tank granuloma, swimming-pool granuloma), Borderline lepromatous leprosy, Borderline leprosy, Borderline tuberculoid, leprosy, Buruli ulcer (Bairnsdale ulcer, Searl ulcer, Searle's ulcer), Erythema induratum (Bazin disease), Histoid leprosy, Lepromatous leprosy, Leprosy (Hansen's disease), Lichen scrofulosorum (Tuberculosis cutis lichenoides), Lupus vulgaris (tuberculosis luposa), Miliary tuberculosis (disseminated tuberculosis, tuberculosis cutis acuta generalisata, tuberculosis cutis disseminata), Mycobacterium avium-intracellulare complex infection, Mycobacterium haemophilum infection, Mycobacterium kansasii infection, Papulonecrotic tuberculid, Primary inoculation tuberculosis (cutaneous primary complex, primary tuberculous complex, Tuberculous chancre), Rapid-growing Mycobacterium infection, Scrofuloderma (Tuberculosis cutis colliquativa), Tuberculosis cutis orificialis (acute tuberculous ulcer, orificial tuberculosis), Tuberculosis verrucosa cutis (lupus verrucosus, prosector's wart, warty tuberculosis), Tuberculous cellulitis, Tuberculous gumma (metastatic tuberculous abscess, metastatic tuberculous ulcer), Tuberculoid leprosy, and sexually transmitted diseases caused by bacteria. Non-limiting examples of sexually transmitted diseases that comprise a microbial infection include Chancroid, Chlamydia, Gonorrhea, Lymphogranuloma Venereum, Mycoplasma genitalium, Nongonococcal Urethritis, Pelvic Inflammatory Disease, Syphilis, vaginitis, bacterial vaginitis, yeast vaginitis, yeast infection.

In another embodiment, the microbial infection is a fungal infection. Non-limiting examples of infectious fungi causing fungal infections that are contemplated for use with the combinatorial therapeutic compositions and methods described herein include, but are not limited to: Candida spp.; Cryptococcus spp.; Aspergillus spp.; Microsporum spp.; Trichophyton spp.; Epidermophyton spp.; Trichosporon spp.; Tinea versicolor; Tinea barbae; Tinea corporis; Tinea cruris; Tinea manuum; Tinea pedis; Tinea unguium; Tinea faciei; Tinea imbricate; Tinea incognito; Epidermophyton floccosum; Microsporum canis; Microsporum audouinii; Trichophyton interdigitale; Trichophyton mentagrophytes; Trichophyton tonsurans; Trichophyton schoenleini; Trichophyton rubrum; Hortaea werneckii; Piedraia hortae; Malasserzia furfur; Coccidioides immitis; Coccidioides posadasii; Histoplasma capsulatum; Histoplasma duboisii; Lacazia loboi; Paracoccidioides brasiliensis; Blastomyces dermatitidis; Sporothrix schenckii; Penicillium marneffei; Candida albicans; Candida glabrata; Candida tropicalis; Candida lusitaniae; Candida jirovecii; Exophiala jeanselmei; Fonsecaea pedrosoi; Fonsecasea compacta; Phialophora verrucosa; Geotrichum candidum; Pseudallescheria boydii; Rhizopus oryzae; Muco indicus; Absidia corymbifera; Synceplasastrum racemosum; Basidiobolus ranarum; Conidiobolus coronatus; Conidiobolus incongruous; Cryptococcus neoformans; Enterocytozoan bieneusi; Encephalitozoon intestinalis; and Rhinosporidium seeberi.

Non-limiting examples of disorders/diseases caused by fungal infections or toxins produced during fungal infections, and for which the compositions and methods described herein are applicable in various aspects and embodiments, include, but are not limited to, infection of a surface wound or burn; infection of a mucosal surface; respiratory infection; infections of the eyes, ears, nose, or throat; or infection of an intestinal pathogen. In other embodiments, the fungal infection is an infection of soft tissue or skin, such as a superficial mycosis; a cutaneous mycosis; a subcutaneous mycosis; a vaginal mycosis; a systemic mycosis; or is an infected wound or burn.

Other medically relevant microorganisms have been described extensively in the literature, e.g., see C. G. A Thomas, Medical Microbiology, Bailliere Tindall, Great Britain 1983, the entire contents of which is hereby incorporated by reference. Each of the foregoing lists is illustrative and is not intended to be limiting.

### Viral Infections

The immunostimulatory oligonucleotide duplexes as described herein can be used to treat a viral infection.

In one aspect, described herein is a method of inducing an anti-viral response in a subject, the method comprising administering to a subject an immunostimulatory oligonucleotide duplex as described herein.

In another aspect, described herein is a method of treating a viral infection in a subject.

In some embodiments, the viral infection is an infection of a tissue selected from the group consisting of central nervous system tissue, eye tissue, upper respiratory system tissue, lower respiratory system tissue, lung tissue, kidney tissue, bladder tissue, spleen tissue, cardiac tissue, gastrointestinal tissue, epidermal tissue, reproductive tissue, nasal cavity tissue, larynx tissue, trachea tissue, bronchi tissue, oral cavity tissue, blood tissue, and muscle tissue.

Non-limiting examples of viral infections include respiratory infections of the nose, throat, upper airways, and lungs such as influenza, pneumonia, coronavirus, SARS, COVID 19, bronchiolitis, and laryngotracheobronchitis; gastrointestinal infections such as gastroenteritis, rotavirus, norovirus; liver infections such as hepatitis; nervous system infections such as rabies, West Nile virus, encephalitis, meningitis, and polio; skin infections such as warts, blemishes, and chickenpox; placental and fetal viral infections such as Zika virus, Rubella virus, and cytomegalovirus; enteroviruses, coxsackieviruses; echoviruses, chikungunya virus, Crimean-Congo hemorrhagic fever virus, Japanese encephalitis virus, Rift Valley Fever virus, Ross River virus, louping ill virus, John Cunningham virus, measles virus, lymphocytic choriomeningitis virus, arbovirus, rhinovirus, parainfluenza virus, respiratory syncytial virus, herpes simplex virus, herpes simplex type 1, herpes simplex type 2, human herpesvirus 6, adenovirus, cytomegalovirus, Epstein-Barr virus, mumps virus, influenza virus type A, influenza virus type B, coronavirus, SARS coronavirus, SARS-CoV-2 virus, coxsackie A virus, coxsackie B virus, poliovirus, HTLV-1, hepatitis virus types A, B, C, D, and E, varicella zoster virus, smallpox virus, molluscum contagiosum, human papillomavirus, parvovirus B19, rubella virus, human immunodeficiency virus, rotavirus, norovirus, astrovirus, ebola virus, Marburg virus, dengue virus (DENV), and Zika virus.

Risk factors for having or developing a viral infection include exposure to the virus, exposure or contact with a subject infected with a virus, exposure to contaminated surfaces contacted with a virus, contact with a biological sample or bodily fluid from a subject infected by a virus, sexual intercourse with a subject infected by a virus, needle sharing, blood transfusions, drug use, and any other risk factor known in the art to transmit a virus from one subject to another. Risk factors for a subject can be evaluated, e.g., by a skilled clinician or by the subject.

### Identification of Microbial Infections

In one embodiment, a subject is diagnosed with having a microbial infection prior to administration of an immunostimulatory oligonucleotide duplex described herein. In another embodiment, the method comprises a step of diagnosing the subject as having a viral infection. In another embodiment, prior to administering, the method comprises a step of receiving results of an assay that diagnoses the subject as having a viral infection or as being at risk of having a viral infection.

There are various tests known to those skilled in the art that are performed in a laboratory to establish or confirm the diagnosis of a microbial infection, as well as to identify the causative microbial species. Common viral infections can be diagnosed based on symptoms, e.g., measles, rubella, chicken pox. The symptoms associated with viral infection vary depending on the type of virus. For example, for an upper respiratory viral infection symptoms include but are not limited to coughing; shortness of breath; fever; and malaise.

For infections that occur in epidemics (e.g., COVID 19 and influenza), the presence of other similar cases may help doctors identify a particular infection. Laboratory diagnosis is important for distinguishing between different viruses that cause similar symptoms, such as COVID-19 (SARS-CoV2) and influenza.

Culturing of microbial species with antimicrobial sensitivity testing is considered the gold standard laboratory test for some microbes. Skin or mucosal samples can be collected in the following ways: 1) dry sterile cotton-tip swab rubbed on the infection site, 2) moist swab taken from a mucosal surface, such as inside the mouth; 3) aspiration of fluid/pus from a skin lesion using a needle and syringe; and 4) skin biopsy: a small sample of skin removed under local anesthetic. Culturing of, e.g., bacteria is most commonly done by brushing the skin swab on sheep blood agar plates and exposing them to different conditions. The species of microbe that grow depend on the medium used to culture the specimen, the temperature for incubation, and the amount of oxygen available. For example, an obligate aerobe can only grow in the presence of oxygen, while an obligate anaerobe cannot grow at all in the presence of oxygen.

Blood tests require a sample of blood accessed by a needle from a vein. Non-limiting examples of tests for microbial infections include: 1) full blood count, infection often raises the white cell count with increased neutrophils (neutrophilia); 2) C-reactive protein (CRP), CRP is often elevated >50 in serious infections; 3) procalcitonin, a marker of generalized sepsis due to bacterial infection, 3) serology, tests 10 days apart to determine immune response to a particular organism; 4) Rapid Plasma Reagin (RPR) test, if syphilis is suspected; and 4) blood culture to detect if high fever >100.4° F. Blood tests can be performed to identify antibodies generated in the presence of a microbial infection.

Polymerase chain reaction (PCR) involves isolating and amplifying lengths of microbial DNA from a sample of skin, blood, or other tissue. The DNA of the sample is compared to DNA from known organisms, thus identifying the species.

### Treatments for Microbial Infections

A number of medications for the treatment of an infection (e.g., a bacterial or viral infection) have been developed. Treatments for infections can include, for example, antibiotics and antiviral medications administered following infection.

The term “therapeutic agent” is art-recognized and refers to any chemical moiety that is a biologically, physiologically, or pharmacologically active substance that acts locally or systemically in a subject. Examples of therapeutic agents, also referred to as “drugs”, are described in well-known literature references such as the Merck Index, the Physicians' Desk Reference, and The Pharmacological Basis of Therapeutics, and they include, without limitation, medications; vitamins; mineral supplements; substances used for the treatment, prevention, diagnosis, cure or mitigation of a disease or illness; substances which affect the structure or function of the body; or pro-drugs, which become biologically active or more active after they have been placed in a physiological environment. Various forms of a therapeutic agent may be used which are capable of being released from the subject composition into adjacent tissues or fluids upon administration to a subject.

Exemplary therapeutic agents and vaccines for the prevention and treatment of infections include but are not limited to penicillin, ceftriaxone, azithromycin, amoxicillin, doxycycline, cephalexin, ciprofloxacin, clindamycin, metronidazole, azithromycin, sulfamethoxazole, trimethoprim, meningococcal polysaccharide vaccine, tetanus toxoid, cholera vaccine, typhoid vaccine, pneumococcal 7-valent vaccine, pneumococcal 13-valent vaccine, pneumococcal 23-valent vaccine, Haemophilus b conjugate, anthrax vaccine, imunovir, indinavir, inosine, lopinavir, lovaride, maravirox, nevirapine, nucleoside analogues, oseltamivir, penciclovir, rimantidine, pyrimidine, saquinavir, stavudine, tenofovir, trizivir, tromantadine, truvada, valaciclovir, ciramidine, zanamivir, zidovudine, MMR vaccine, DTaP vaccine, hepatitis vaccines, Hib vaccine, HPV vaccine, influenza vaccine, polio vaccine, rotavirus vaccine, shingles vaccine, Tdap vaccine, tetanus vaccine, fluconazole, ketoconazole, amphotericin B, and sulfadoxine/pyrimethamine. Additional non-limiting examples include Abacavir, Acyclovir (Aciclovir), Adefovir, Amantadine, Ampligen, Amprenavir (Agenerase), Amodiaquine, Apilimod, Arbidol, Atazanavir, Atripla, Balavir, Baloxavir marboxil (Xofluza®), Biktarvy Boceprevir (Victrelis®), Cidofovir, Clofazimine, Clomifene, Cobicistat (Tybost®), Combivir (fixed dose drug), Daclatasvir (Daklinza®), Darunavir, Delavirdine, Descovy, Didanosine, Docosanol, Dolutegravir, Doravirine (Pifeltro®), Ecoliever, Edoxudine, Efavirenz, Elvitegravir, Emtricitabine, Enfuvirtide, Entecavir, Etravirine (Intelence®), Famciclovir, Favipiravir, Fenofibrate, Fomivirsen, Fosamprenavir, Foscarnet, Fosfonet, Fusion inhibitor, Ganciclovir (Cytovene®), Ibacitabine, Ibalizumab (Trogarzo®), Idoxuridine, Imiquimod, Imunovir, Indinavir, Inosine, Integrase inhibitor, Interferon type I, Interferon type II, Interferon type III, Interferon, Lamivudine, Letermovir (Prevymis®), Lopinavir, Loviride, Mannose Binding Lectin, Maraviroc, Methisazone, Moroxydine, Nafamostat, Nelfinavir, Nevirapine, Nexavir®, Nilotinib, Nitazoxanide, Norvir, Nucleoside analogues, Oseltamivir (Tamiflu®), Pazopanib, Peginterferon alfa-2a, Peginterferon alfa-2b, Penciclovir, Peramivir (Rapivab®), Pleconaril, Podophyllotoxin, Protease inhibitor (pharmacology), Pyramidine, Raltegravir, Remdesivir, Reverse transcriptase inhibitor, Ribavirin, Rilpivirine (Edurant®), Rimantadine, Ritonavir, Saquinavir, Simeprevir (Olysio®), Sofosbuvir, Stavudine, Synergistic enhancer (antiretroviral), Telaprevir, Telbivudine (Tyzeka®), Tenofovir alafenamide, Tenofovir disoproxil, Tenofovir, Toremifene, Tipranavir, Trifluridine, Trizivir, Tromantadine, Truvada, Valaciclovir (Valtrex), Valganciclovir, Vicriviroc, Vidarabine, Viramidine, Zalcitabine, Zanamivir (Relenza®), and Zidovudine.

In some embodiments of any of the aspects, the immunostimulatory oligonucleotide duplexes described herein are used as a monotherapy.

In another embodiment of any of the aspects, the compositions described herein can be used in combination with other known compositions and therapies for an interferon-mediated disease (e.g., autoimmune disease, infection, or cancer). The immunostimulatory oligonucleotide duplexes described herein can be e.g., in admixture with an antiviral therapeutic or administered as a therapeutic regimen for the treatment of an interferon-mediated disease.

Administered “in combination,” as used herein, means that two (or more) different treatments are delivered to the subject during the course of the subject's affliction with the disorder, e.g., the two or more treatments are delivered after the subject has been diagnosed with the disorder (a respiratory disease) and before the disorder has been cured or eliminated or treatment has ceased for other reasons. Non-limiting examples of treatments that can be used in combination with the compositions provided herein include Abacavir, Acyclovir (Aciclovir), Adefovir, Amantadine, Ampligen, Amprenavir (Agenerase), Amodiaquine, Apilimod, Arbidol, Atazanavir, Atripla, Atovaquone, Balavir, Baloxavir marboxil (Xofluza®), Biktarvy Boceprevir (Victrelis®), Cidofovir, Clofazimine, Clomifene, Clofazamine, Cobicistat (Tybost®), Combivir (fixed dose drug), Daclatasvir (Daklinza®), Darunavir, Delavirdine, Descovy, Didanosine, Docosanol, Dolutegravir, Doravirine (Pifeltro®), Ecoliever, Edoxudine, Efavirenz, Elvitegravir, Emtricitabine, Enfuvirtide, Entecavir, Etravirine (Intelence®), Famciclovir, Favipiravir, Fenofibrate, Fomivirsen, Fosamprenavir, Foscarnet, Fosfonet, Fusion inhibitor, Ganciclovir (Cytovene®), Ibacitabine, Ibalizumab (Trogarzo®), Idoxuridine, Imiquimod, Imunovir, Indinavir, Inosine, Integrase inhibitor, Interferon type I, Interferon type II, Interferon type III, Interferon, Ivermectin, Lamivudine, Lasalocid, Letermovir (Prevymis®), Lopinavir, Loviride, Mannose Binding Lectin, Maraviroc, Methisazone, Moroxydine, Nafamostat, Nelfinavir, Nevirapine, Nexavir®, Nilotinib, Nitazoxanide, Norvir, Nucleoside analogues, Oseltamivir (Tamiflu®), Pazopanib, Peginterferon alfa-2a, Peginterferon alfa-2b, Penciclovir, Peramivir (Rapivab®), Pleconaril, Podophyllotoxin, Protease inhibitor (pharmacology), Pyonaridine, Pyramidine, Raltegravir, Remdesivir, Reverse transcriptase inhibitor, Ribavirin, Rilpivirine (Edurant®), Rimantadine, Ritonavir, Saquinavir, Simeprevir (Olysio®), Sofosbuvir, Stavudine, Synergistic enhancer (antiretroviral), Tafenoquine, Telaprevir, Telbivudine (Tyzeka®), Tenofovir alafenamide, Tenofovir disoproxil, Tenofovir, Toremifene, Tipranavir, Trifluridine, Trizivir, Tromantadine, Truvada, Valaciclovir (Valtrex), Valganciclovir, Vermurafenib, Venetoclax, Vicriviroc, Vidarabine, Viramidine, Zalcitabine, Zanamivir (Relenza®), and Zidovudine.

In some embodiments, the immunostimulatory oligonucleotide duplex and the at least one antiviral therapeutic are administered at substantially the same time.

In some embodiments, the at least one antiviral therapeutic are administered at different time points.

In some embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered. The compositions described herein and the at least one additional therapy can be administered simultaneously, in the same or in separate compositions, or sequentially. For sequential administration, the composition described herein can be administered first, and the additional composition can be administered second, or the order of administration can be reversed. The composition and/or other therapeutic compositions, procedures or modalities can be administered during periods of active disorder, or during a period of remission or less active disease. The composition can be administered before another treatment, concurrently with the treatment, post-treatment, or during remission of the disorder.

When administered in combination, the composition and the additional agent or composition (e.g., second or third agent), or all, can be administered in an amount or dose that is higher, lower or the same as the amount or dosage of each agent used individually, e.g., as a monotherapy. In certain embodiments, the administered amount or dosage of the agent, the additional agent (e.g., second or third agent), or all, is lower (e.g., at least 20%, at least 30%, at least 40%, or at least 50%) than the amount or dosage of each agent used individually. In other embodiments, the amount or dosage of agent, the additional agent (e.g., second or third agent), or all, that results in a desired effect (e.g., treatment of a respiratory disease) is lower (e.g., at least 20%, at least 30%, at least 40%, or at least 50% lower) than the amount or dosage of each agent individually required to achieve the same therapeutic effect.

A vaccine composition as described herein can be used, for example, to protect or treat a subject against disease. The terms “immunize” and “vaccinate” tend to be used interchangeably in the field. However, in reference to the administration of the vaccine compositions as described herein to provide protection against disease, e.g., infectious disease caused by a pathogen that expresses the antigen, it should be understood that the term “immunize” refers to the passive protection conferred by the administered vaccine composition.

### Administration, Dosage, and Efficacy

The immunostimulatory oligonucleotide duplex, pharmaceutical composition, or vaccine compositions described herein can be formulated, dosed, and administered in a fashion consistent with good medical practice. Factors for consideration in this context include the particular disorder being treated, the particular subject being treated, the clinical condition of the individual subject, the cause of the disorder, the site of delivery of the vaccine composition, the method of administration, the scheduling of administration, and other factors known to medical practitioners.

The therapeutic formulations to be used for in vivo administration, such as parenteral administration, in the methods described herein can be sterile, which is readily accomplished by filtration through sterile filtration membranes, or other methods known to those of skill in the art.

The immunostimulatory oligonucleotide duplexes and compositions thereof as described herein can be administered to a subject in need thereof by any appropriate route which results in an effective treatment in the subject. As used herein, the terms “administering,” and “introducing” are used interchangeably and refer to the placement of a vaccine composition, antigen or fragment thereof into a subject by a method or route which results in at least partial localization of such vaccine compositions at a desired site, such as a site of infection, such that a desired effect(s) is produced. An antigen or fragment thereof or vaccine composition can be administered to a subject by any mode of administration that delivers the vaccine composition systemically or to a desired surface or target, and can include, but is not limited to, injection, infusion, instillation, and inhalation administration. To the extent that antigen or fragment thereof or vaccine composition can be protected from inactivation in the gut, oral administration forms are also contemplated. “Injection” includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion.

The phrases “parenteral administration” and “administered parenterally” as used herein, refer to modes of administration other than enteral and topical administration, usually by injection. The phrases “systemic administration,” “administered systemically”, “peripheral administration” and “administered peripherally” as used herein refer to the administration of a therapeutic agent other than directly into a target site, tissue, or organ, such as a tumor site, such that it enters the subject's circulatory system and, thus, is subject to metabolism and other like processes. In other embodiments, the antibody or antigen-binding fragment thereof is administered locally, e.g., by direct injections, when the disorder or location of the infection permits, and the injections can be repeated periodically.

In some embodiments, the compositions described herein are administered by aerosol administration, nebulizer administration, or tracheal lavage administration. In some embodiments, the composition is formulated for intravenous, intramuscular, intraperitoneal, subcutaneous, or intrathecal administration.

The term “effective amount” as used herein refers to the amount of an immunostimulatory oligonucleotide duplex composition needed to alleviate or prevent at least one or more symptom of an infection, disease or disorder, and relates to a sufficient amount of pharmacological composition to provide the desired effect, e.g., reduce the level of pathogenic microorganisms at a site of infection, reduce pathology, or any symptom associated with or caused by the pathogenic microorganism. The term “therapeutically effective amount” therefore refers to an amount of an antigen or fragment thereof or vaccine composition described herein using the methods as disclosed herein, that is sufficient to effect a particular effect when administered to a typical subject. An effective amount as used herein would also include an amount sufficient to delay the development of a symptom of the disease, alter the course of a symptom disease (for example, but not limited to, slow the progression of a symptom of the disease), or reverse a symptom of the disease. Thus, it is not possible to specify the exact “effective amount.” However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the antigen or fragment thereof), which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay. The dosage can be determined by a physician and adjusted, as necessary, to suit observed effects of the treatment.

The immunostimulatory oligonucleotide duplexes, pharmaceutical compositions, or vaccine compositions described herein can be formulated, in some embodiments, with one or more additional therapeutic agents currently used to prevent or treat the infection, for example. The effective amount of such other agents depends on the amount of immunostimulatory oligonucleotide duplex in the formulation, the type of disorder or treatment, and other factors discussed above. These are generally used in the same dosages and with administration routes as used herein before or about from 1 to 99% of the heretofore employed dosages.

The dosage ranges for the immunostimulatory oligonucleotide duplexes, pharmaceutical composition, or vaccine compositions described herein depend upon the potency, and encompass amounts large enough to produce the desired effect. The dosage should not be so large as to cause unacceptable adverse side effects. Generally, the dosage will vary with the age, condition, and sex of the patient and can be determined by one of skill in the art. The dosage can also be adjusted by the individual physician in the event of any complication. In some embodiments, the dosage ranges from 0.001 mg/kg body weight to 100 mg/kg body weight. In some embodiments, the dose range is from 5 g/kg body weight to 100 μg/kg body weight. Alternatively, the dose range can be titrated to maintain serum levels between 1 μg/mL and 1000 μg/mL. For systemic administration, subjects can be administered a therapeutic amount, such as, e.g., 0.1 mg/kg, 0.5 mg/kg, 1.0 mg/kg, 2.0 mg/kg, 2.5 mg/kg, 5 mg/kg, 7.5 mg/kg, 10 mg/kg, 15 mg/kg, 20 mg/kg, 25 mg/kg, 30 mg/kg, 40 mg/kg, 50 mg/kg, or more. These doses can be administered by one or more separate administrations, or by continuous infusion. For repeated administrations over several days or longer, depending on the condition, the treatment is sustained until, for example, the infection is treated, as measured by the methods described above or known in the art. However, other dosage regimens can be useful.

The duration of a therapy using the methods described herein will continue for as long as medically indicated or until a desired therapeutic effect (e.g., those described herein) is achieved. In certain embodiments, the administration of the vaccine composition described herein is continued for 1 month, 2 months, 4 months, 6 months, 8 months, 10 months, 1 year, 2 years, 3 years, 4 years, 5 years, 10 years, 20 years, or for a period of years up to the lifetime of the subject.

As will be appreciated by one of skill in the art, appropriate dosing regimens for a given composition can comprise a single administration/immunization or multiple ones. Subsequent doses may be given repeatedly at time periods, for example, about two weeks or greater up through the entirety of a subject's life, e.g., to provide a sustained preventative effect. Subsequent doses can be spaced, for example, about two weeks, about three weeks, about four weeks, about one month, about two months, about three months, about four months, about five months, about six months, about seven months, about eight months, about nine months, about ten months, about eleven months, or about one year after a primary immunization.

The precise dose to be employed in the formulation will also depend on the route of administration and should be decided according to the judgment of the practitioner and each patient's circumstances. Ultimately, the practitioner or physician will decide the amount of the immunostimulatory oligonucleotide duplex or composition thereof to administer to particular subjects.

In some embodiments of these methods and all such methods described herein, the immunostimulatory oligonucleotide duplex or composition thereof is administered in an amount effective to provide short-term protection against an infection or to treat an infection. In some embodiments, the infection is a viral infection. As used herein, “short-term protection” refers to protection from an infection, such as a malarial infection, lasting at least about 2 weeks, at least about 1 month, at least about 6 weeks, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. Such protection can involve repeated dosing.

In some embodiments of these methods and all such methods described herein, the immunostimulatory oligonucleotide duplex or composition thereof is administered in an amount effective to provide protection against an infection or to alleviate a symptom of a persistent infection.

“Alleviating a symptom of a persistent infection” is ameliorating any condition or symptom associated with the persistent infection. Alternatively, alleviating a symptom of a persistent infection can involve reducing the infectious microbial (such as viral, bacterial, fungal or parasitic) load in the subject relative to such load in an untreated control. As compared with an equivalent untreated control, such reduction or degree of prevention is at least 5%, 10%, 20%, 40%, 50%, 60%, 80%, 90%, 95%, or 100% as measured by any standard technique. Desirably, the persistent infection is completely cleared as detected by any standard method known in the art, in which case the persistent infection is considered to have been treated.

A patient who is being treated for a persistent infection is one who a medical practitioner has diagnosed as having such a condition. Diagnosis may be by any suitable means. Diagnosis and monitoring may involve, for example, detecting the level of microbial load in a biological sample (for example, a tissue biopsy, blood test, or urine test), detecting the level of a surrogate marker of the microbial infection in a biological sample, detecting symptoms associated with persistent infections, or detecting immune cells involved in the immune response typical of persistent infections (for example, detection of antigen specific T cells that are anergic and/or functionally impaired). A patient in whom the development of a persistent infection is being prevented may or may not have received such a diagnosis. One in the art will understand that these patients may have been subjected to the same standard tests as described above or may have been identified, without examination, as one at high risk due to the presence of one or more risk factors (such as family history or exposure to infectious agent).

All patents and other publications identified are expressly incorporated herein by reference for the purpose of describing and disclosing, for example, the methodologies described in such publications that might be used in connection with the present disclosure. These publications are provided solely for their disclosure prior to the filing date of the present application. Nothing in this regard should be construed as an admission that the inventors are not entitled to antedate such disclosure by virtue of prior disclosure or for any other reason. All statements as to the date or representation as to the contents of these documents are based on the information available to the applicants and do not constitute any admission as to the correctness of the dates or contents of these documents.

It should be understood that this disclosure is not limited to the particular methodology, protocols, and reagents, etc., provided herein and as such may vary. The terminology used herein is for the purpose of describing particular embodiments only, and is not intended to limit the scope of the present disclosure, which is defined solely by the claims. The invention is further illustrated by the following example, which should not be construed as further limiting.

The technology can further be described in the following numbered paragraphs:
1. An immunostimulatory oligonucleotide duplex comprising SEQ ID NO:1 at a 5′ end.
2. The immunostimulatory oligonucleotide duplex of paragraph 1, wherein the oligonucleotide duplex is RNA.
3. The immunostimulatory oligonucleotide duplex of any of the preceding paragraphs, wherein the oligonucleotide duplex comprises a 5′-monophosphate group
4. The immunostimulatory oligonucleotide duplex of any of the preceding paragraphs, wherein the oligonucleotide duplex is at least 20 nucleobases in length.
5. The immunostimulatory oligonucleotide duplex of any of the preceding paragraphs, wherein the oligonucleotide duplex is double stranded RNA.
6. The immunostimulatory oligonucleotide duplex of any of the preceding paragraphs, wherein the oligonucleotide duplex is sufficient to induce interferon (IFN) production in a cell contacted with the duplex.
7. The method of any of the preceding paragraphs, wherein the IFN production is type I IFN production.
8. The immunostimulatory oligonucleotide duplex of any of the preceding paragraphs, wherein the oligonucleotide duplex activates the RIG-I-IRF3 pathway.
9. The immunostimulatory oligonucleotide duplex any of the preceding paragraphs, wherein the oligonucleotide duplex reduces a viral titer or viral load in a cell or population of cells contacted with the duplex.
10. The immunostimulatory oligonucleotide duplex any of the preceding paragraphs, wherein the oligonucleotide duplex increases STAT1 and STAT2 in a cell contacted by the duplex.
11. A method of inducing an anti-viral response is a subject, the method comprising administering to a subject in need thereof an immunostimulatory oligonucleotide duplex of any of the preceding paragraphs.
12. A method of treating a viral infection in a subject, the method comprising administering to a subject in need thereof an immunostimulatory oligonucleotide duplex of any of the preceding paragraphs.
13. The method of any of the preceding paragraphs, wherein the subject in need thereof has a viral infection, or is at risk of having a viral infection.
14. The method of any of the preceding paragraphs, further comprising, prior to administering, a step of diagnosing the subject as having a viral infection or being at risk of having a viral infection.
15. The method of any of the preceding paragraphs, further comprising, prior to administering, a step of receiving results of an assay that diagnoses the subject as having a viral infection or as being at risk of having a viral infection.
16. The method of any of the preceding paragraphs, wherein the viral infection is caused by a virus selected from the group consisting of: John Cunningham virus, measles virus, Lymphocytic choriomeningitis virus, arbovirus, rabies virus, rhinovirus, parainfluenza virus, respiratory syncytial virus, herpes simplex virus, herpes simplex type 1, herpes simplex type 2, human herpesvirus 6, adenovirus, cytomegalovirus, Epstein-Barr virus, mumps virus, influenza virus type A, influenza virus type B, coronavirus, SARS coronavirus, SARS-CoV-2 virus, coxsackie A virus, coxsackie B virus, poliovirus, HTLV-1, hepatitis virus types A, B, C, D, and E, varicella zoster virus, smallpox virus, molluscum contagiosum, human papillomavirus, parvovirus B19, rubella virus, human immunodeficiency virus, rotavirus, norovirus, astrovirus, ebola virus, Marburg virus, dengue virus (DENV), and Zika virus.
17. The method of any of the preceding paragraphs, wherein the viral infection is an infection of a tissue selected from the group consisting of central nervous system tissue, eye tissue, upper respiratory system tissue, lower respiratory system tissue, lung tissue, kidney tissue, bladder tissue, spleen tissue, cardiac tissue, gastrointestinal tissue, epidermal tissue, reproductive tissue, nasal cavity tissue, larynx tissue, trachea tissue, bronchi tissue, oral cavity tissue, blood tissue, and muscle tissue.
18. The method of any of the preceding paragraphs, wherein the administration is systemic.
19. The method of any of the preceding paragraphs, wherein the administration is local at a site of viral infection.
20. The method of any of the preceding paragraphs, further comprising administering at least one additional therapeutic.
21. The method of any of the preceding paragraphs, wherein the at least one additional therapeutic is an anti-viral therapeutic.
22. A method of treating an influenza infection in a subject, the method comprising administering to a subject having an influenza infection an immunostimulatory oligonucleotide duplex of any of the preceding paragraphs.
23. The method of any of the preceding paragraphs, wherein the influenza infection is an influenza A infection, or an influenza B infection.
24. The method of any of the preceding paragraphs, further comprising administering at least one additional anti-viral therapeutic.
25. A method of treating a coronavirus disease in a subject, the method comprising administering to a subject having a coronavirus disease an immunostimulatory oligonucleotide duplex of any of the preceding paragraphs.
26. The method of any of the preceding paragraphs, wherein the coronavirus disease is COVID-19.
27. The method of any of the preceding paragraphs, further comprising administering at least one additional anti-viral therapeutic.
28. The method of any of the preceding paragraphs, further comprising administering plasma obtained from a subject that has recovered from the coronavirus disease.
29. A method of increasing the efficacy of an anti-viral therapeutic, the method comprising administering an immunostimulatory oligonucleotide duplex of any of the preceding paragraphs and at least one anti-viral therapeutic.
30. The method of any of the preceding paragraphs, wherein the anti-viral therapeutic is selected from the group consisting of: Abacavir, Acyclovir (Aciclovir), Adefovir, Amantadine, Ampligen, Amprenavir (Agenerase), Amodiaquine, Apilimod, Arbidol, Atazanavir, Atripla, Atovaquone, Balavir, Baloxavir marboxil (Xofluza®), Biktarvy Boceprevir (Victrelis®), Cidofovir, Clofazimine, Clomifene, Clofazamine, Cobicistat (Tybost®), Combivir (fixed dose drug), Daclatasvir (Daklinza®), Darunavir, Delavirdine, Descovy, Didanosine, Docosanol, Dolutegravir, Doravirine (Pifeltro®), Ecoliever, Edoxudine, Efavirenz, Elvitegravir, Emtricitabine, Enfuvirtide, Entecavir, Etravirine (Intelence®), Famciclovir, Favipiravir, Fenofibrate, Fomivirsen, Fosamprenavir, Foscarnet, Fosfonet, Fusion inhibitor, Ganciclovir (Cytovene®), Ibacitabine, Ibalizumab (Trogarzo®), Idoxuridine, Imiquimod, Imunovir, Indinavir, Inosine, Integrase inhibitor, Interferon type I, Interferon type II, Interferon type III, Interferon, Ivermectin, Lamivudine, Lasalocid, Letermovir (Prevymis®), Lopinavir, Loviride, Mannose Binding Lectin, Maraviroc, Methisazone, Moroxydine, Nafamostat, Nelfinavir, Nevirapine, Nexavir®, Nilotinib, Nitazoxanide, Norvir, Nucleoside analogues, Oseltamivir (Tamiflu®), Pazopanib, Peginterferon alfa-2a, Peginterferon alfa-2b, Penciclovir, Peramivir (Rapivab®), Pleconaril, Podophyllotoxin, Protease inhibitor (pharmacology), Pyonaridine, Pyramidine, Raltegravir, Remdesivir, Reverse transcriptase inhibitor, Ribavirin, Rilpivirine (Edurant®), Rimantadine, Ritonavir, Saquinavir, Simeprevir (Olysio®), Sofosbuvir, Stavudine, Synergistic enhancer (antiretroviral), Tafenoquine, Telaprevir, Telbivudine (Tyzeka®), Tenofovir alafenamide, Tenofovir disoproxil, Tenofovir, Toremifene, Tipranavir, Trifluridine, Trizivir, Tromantadine, Truvada, Valaciclovir (Valtrex), Valganciclovir, Vermurafenib, Venetoclax, Vicriviroc, Vidarabine, Viramidine, Zalcitabine, Zanamivir (Relenza®), and Zidovudine.
31. The method of any of the preceding paragraphs, wherein the immunostimulatory oligonucleotide duplex and the at least one antiviral therapeutic are administered at substantially the same time.
32. The method of any of the preceding paragraphs, wherein the immunostimulatory oligonucleotide duplex and the at least one antiviral therapeutic are administered at different time points.
33. A pharmaceutical composition comprising an immunostimulatory oligonucleotide duplex of any of the preceding paragraphs and a pharmaceutically acceptable carrier.
34. A pharmaceutical composition comprising an immunostimulatory oligonucleotide duplex of any of the preceding paragraphs and at least one anti-viral therapeutic.
35. The composition of any of the preceding paragraphs, wherein the composition is formulated for airway administration.
36. The composition of any of the preceding paragraphs, wherein the composition is formulated for aerosol administration, nebulizer administration, or tracheal lavage administration.
37. A method of inducing interferon (IFN) production, the method comprising administering to a subject in need thereof an immunostimulatory oligonucleotide duplex of any of the preceding paragraphs, or a pharmaceutical composition of any of the preceding paragraphs, whereby IFN production is increased following administration.
38. The method of any of the preceding paragraphs, wherein IFN production is the production of type I IFN, type II IFN, or type III IFN.
39. The method of any of the preceding paragraphs, wherein IFN production is the production of type I IFN.
40. The method of any of the preceding paragraphs, wherein the type I IFN is IFN-α, IFN-β, IFN-ε, IFN-κ or IFN-ω.
41. The method of any of the preceding paragraphs, wherein the type II IFN is IFN-γ.
42. The method of any of the preceding paragraphs, wherein increased IFN production increases cellular resistance to a viral infection.
43. A method of treating an IFN-associated disease, the method comprising administering to a subject in need thereof an immunostimulatory oligonucleotide duplex of any of the preceding paragraphs.
44. The method of any of the preceding paragraphs, wherein the subject in need thereof has an IFN-associated disease, or is at risk of having an IFN-associated disease.
45. The method of any of the preceding paragraphs, further comprising, prior to administering, a step of diagnosing a subject as having an IFN-associated disease or at risk of having an IFN-associated disease.
46. The method of any of the preceding paragraphs, further comprising, prior to administering, receiving the results of an assay that diagnoses a subject as having an IFN-associated disease or at risk of an IFN-associated disease.
47. The method of any of the preceding paragraphs, wherein the IFN-associated disease is a disease involving reduced IFN levels as compared to a reference level.
48. The method of any of the preceding paragraphs, wherein the IFN-associated disease is a disease involving reduced Type I IFN levels as compared to a reference level.
49. The method of any of the preceding paragraphs, wherein the IFN-associated disease is selected from the group consisting of a viral infectious disease, a bacterial infectious disease, a fungal infectious disease, a parasitic infectious disease, cancer, and an autoimmune disease.
50. The method of any of the preceding paragraphs, further comprising administering at least one additional therapeutic.
51. The method of any of the preceding paragraphs, wherein the at least one additional therapeutic is an anti-viral therapeutic, an anti-bacterial therapeutic, an anti-fungal therapeutic, an anti-parasitic therapeutic, an anti-cancer therapeutic, or an anti-autoimmune therapeutic.
52. A composition comprising an immunostimulatory oligonucleotide duplex of any of the preceding paragraphs and at least one anti-bacterial therapeutic.
53. A composition comprising an immunostimulatory oligonucleotide duplex of any of the preceding paragraphs and at least one anti-fungal therapeutic.
54. A composition comprising an immunostimulatory oligonucleotide duplex of any of the preceding paragraphs and at least one anti-parasitic therapeutic.
55. A composition comprising an immunostimulatory oligonucleotide duplex of any of the preceding paragraphs and at least one anti-cancer therapeutic.
56. A composition comprising the immunostimulatory oligonucleotide duplex of any of the preceding paragraphs and at least one anti-autoimmune therapeutic.
57. The composition of any of the preceding paragraphs, further comprising a pharmaceutically acceptable carrier.
58. An immunostimulatory oligonucleotide duplex comprising SEQ ID NO:1 at a 5′ end, conjugated to an antigen or vaccine.
59. A composition comprising an immunostimulatory oligonucleotide duplex of any of the preceding paragraphs.
60. A composition comprising an immunostimulatory oligonucleotide duplex of any of the preceding paragraphs and a vaccine.
61. A composition comprising an immunostimulatory oligonucleotide duplex of any of the preceding paragraphs and a nanoparticle.
62. A nanoparticle comprising an immunostimulatory oligonucleotide duplex of any of the preceding paragraphs.
63. A composition comprising an immunostimulatory oligonucleotide duplex of any of the preceding paragraphs and a nanoparticle.
64. A nanoparticle comprising an immunostimulatory oligonucleotide duplex of any of the preceding paragraphs.
65. The composition of any of the preceding paragraphs, further comprising a pharmaceutically acceptable carrier.
66. A method of vaccinating, the method comprising administering to a subject in need thereof
a. an immunostimulatory oligonucleotide duplex of any of the preceding paragraphs;
b. a composition of any of the preceding paragraphs; or
c. an immunostimulatory oligonucleotide duplex of any of the preceding paragraphs and a vaccine.
67. A method of increasing the efficacy of a vaccine, the method comprising administering to a subject in need thereof
a. the immunostimulatory oligonucleotide duplex of any of the preceding paragraphs;
b. a composition of any of the preceding paragraphs; or
c. an immunostimulatory oligonucleotide duplex of any of the preceding paragraphs and a vaccine.
68. The composition of any of the preceding paragraphs, wherein the composition is formulated for intravenous, intramuscular, intraperitoneal, subcutaneous, or intrathecal administration.

### EXAMPLES

### Example 1: Duplex RNA Interferon Inducer Therapeutics for Pathogenic Infections and Other Immune Response-Related Diseases

The increasing incidence of potentially pandemic viruses, such as influenza, MERS, SARS, and now SARS-CoV-2, requires development of new broad-spectrum therapies that inhibit infection by many different types of viruses. The best way to accomplish this is by targeting the general host response to viral, rather than targeting the viruses themselves. As an example, Influenza A virus is a major human pathogen that causes annual epidemics and occasional pandemics with serious public health and economic impact. Influenza infection and replication in host cells is a multi-step process: the virus binds to host surface receptors and enters the cell, then releases its genome into the cytoplasm. The viral genome is subsequently imported to the nucleus, where viral transcription and replication occur, and the new synthesized viral proteins and RNA assemble into progeny viral particles, which release to the extracellular environment by budding.

Following infection with viruses and other types of pathogens (e.g., bacteria, fungi, parasites), the human body triggers a complex regulatory system of innate and adaptive immune responses designed to defend against the virus. One of the many responses to the viral invasion is the induction of interferon (IFN) production, which is a pleiotropic cytokine that plays a critical role in human immune responses by ‘interfering’ with viral replication¹. Induction of IFN gene expression also leads to increased cellular resistance to viral infection by activate immune cells, (e.g., natural killer cells and macrophages), and increasing host defenses by upregulating antigen presentation by virtue of increasing the expression of major histocompatibility complex (MHC) antigens. There are many types of distinct IFN genes and proteins, which are typically divided among three classes in humans: Type I IFN IFN-α, IFN-β, IFN-ε, IFN-κ and IFN-ω), Type II IFN (IFN-γ), and Type III IFN. IFNs belonging to all three classes are important for fighting viral infections and for the regulation of the immune system. IFN also has been used as a therapeutic in the treatment of multiple sclerosis, many types of cancer, and virus infection.

Increased production of IFNs can inhibit infection by influenza and many other types of viruses, but this potential therapeutic route might be particularly useful for treatment of COVID-19. This is because compared to the response to influenza A virus and respiratory syncytial virus, the virus that causes COVID-19 (SARS-CoV-2) elicits a muted response that lacks robust induction of a subset of cytokines, including the Type I and Type III IFNs, while continuing to produce other inflammatory cytokines that can lead to the cytokine storm that is the cause of mortality in many patients².

Described herein are specific duplex RNA sequences that activate the interferon pathway, up-regulate expression of Type I IFNs and decrease influenza A viral infection when transfected into human A549 lung epithelial cells. The duplex RNA sequences identified herein also result in large (100-fold) reductions of influenza A viral infection when transfected into human A549 lung epithelial cells.

Screening for lncRNAs that Mediate Influenza Virus Infection

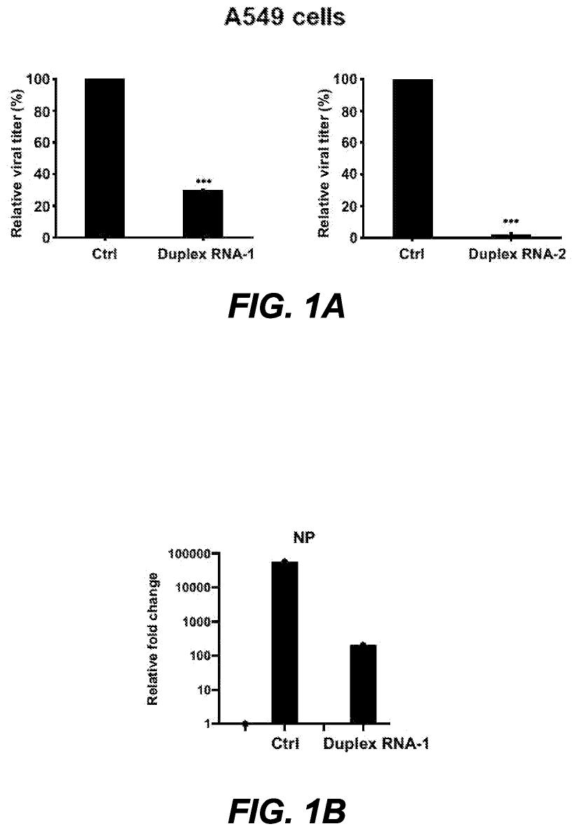

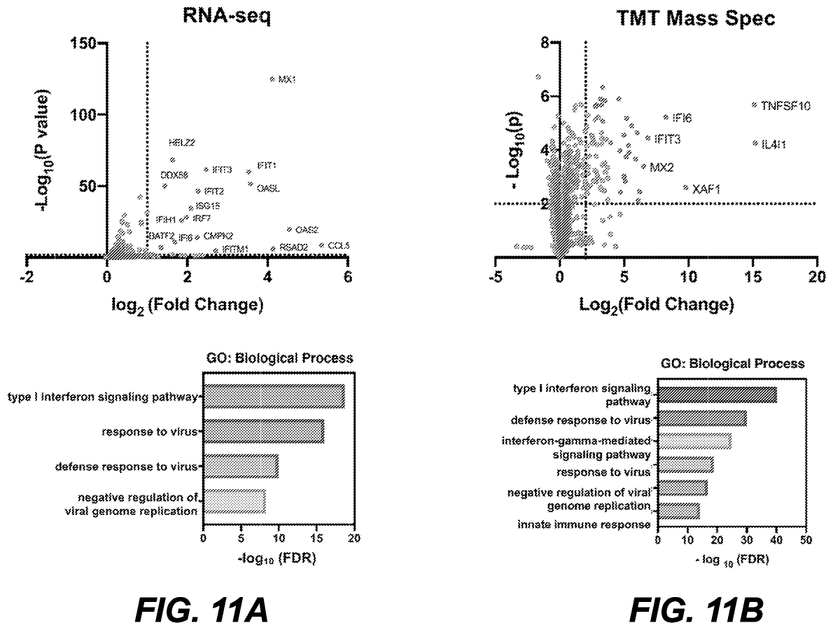

Described herein is a CRISPR/Cas9-based screening strategy to identify lncRNAs that mediate influenza virus infection. Cells harboring sgRNAs that knockout lncRNAs that confer cells resistant to influenza infection, but do not affect cell growth, can survive and expand rapidly. After deep sequencing, enriched lncRNAs were identified using a Model-based Analysis of Genome-wide CRISPR/Cas9 Knockout (MAGeCK) method for prioritizing sgRNAs, genes, and pathways in genome-scale CRISPR/Cas9 knockout screens. Hundreds of Dicer-Substrate Short Interfering RNAs (DsiRNAs) were tested that target the relevant lncRNA sequences by transfecting them into human A549 lung epithelial cells and then infecting these cells with influenza virus. This analysis resulted in the discovery that transfection of two of these DsiRNAs targeting lnc RNAs DGCR5 (duplex RNA-1) and LINC00261 (duplex RNA-2) suppressed influenza infection by ˜80% and ˜98 in (FIG. 1A). About a 100-fold inhibition of virus titers was observed when the same experiment was carried out in the influenza-infected human Lung Airway Chips (FIG. 11B), which more closely mimics human lung airway pathophysiology³⁻⁵.

The siRNAs with a Common Sequence Induced Interferon Directly, not Via lncRNAs

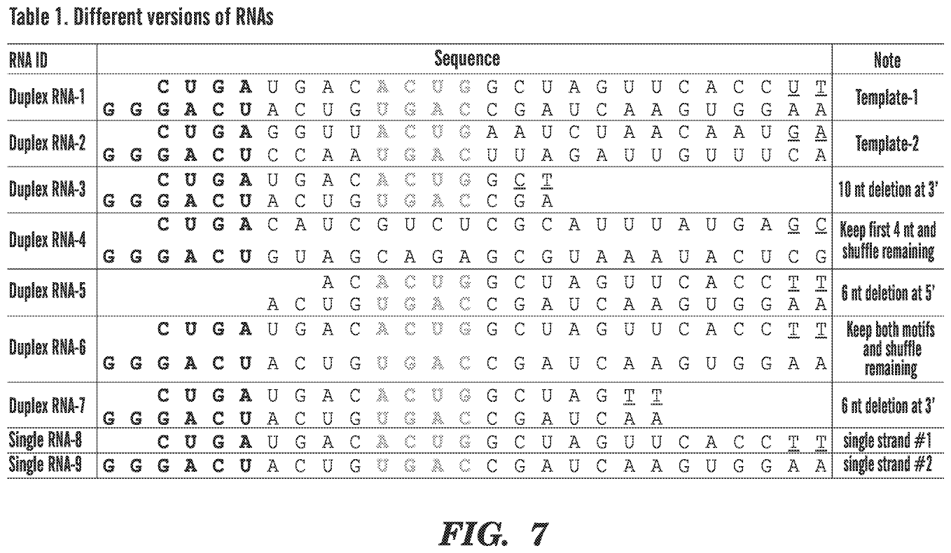

Importantly, when additional studies were carried using multiple DsiRNAs against DGCR5 and LINC00261 to further validate the function of DGCR5 and LINC00261, it was surprisingly discovered that only a subset of siRNAs could both knock down DGCR5 or LINC00261 and induce IFN production; the others specifically knocked down DGCR5 or LINC00261 as designed, but they did not induce IFN production. Even more importantly, the active siRNA that induced IFN production all contained the same sequences (shown in gray and light gray Table 1, FIG. 7), even though they were designed to target different lncRNAs. It is important to note that these double stranded (Duplex) RNAs contain sequence-encoded structures that were specifically designed not to induce interferon induction as shown by others⁶. Thus, the common sequences found in both of these duplex RNAs (duplex RNA-1 and -2) shown in Table 1 (FIG. 7) appear to specifically induce IFN production based on their specific nucleotide sequence composition rather than by suppressing lncRNA expression.

TABLE 1
Duplex and single RNAs

| RNA ID | SEQ ID NOs: | SEQUENCES 5′ > 3′ |
|---|---|---|
| Duplex | SEQ ID NO: 3 | CUGAUGACACUGGCUAGUUCACCUT |
| RNA-1 | SEQ ID NO: 4 | GGGACUACUGUGACCGAUCAAGUGGAA |
| Duplex | SEQ ID NO: 5 | CUGAGGUUACUGAAUCUAACAAUGA |
| RNA-2 | SEQ ID NO: 6 | GGGACUCCAAUGACUUAGAUUGUUUCA |
| Duplex | SEQ ID NO: 7 | CUGAUGACACUGGCT |
| RNA-3 | SEQ ID NO: 8 | GGGACUACUGUGACCGA |
| Duplex | SEQ ID NO: 9 | CUGACAUCGUCUCGCAUUUAUGAGC |
| RNA-4 | SEQ ID NO: 51 | GGGACUGUAGCAGAGCGUAAAU |
| Duplex | SEQ ID NO: 11 | ACACUGGCUAGUUCACCTT |
| RNA-5 | SEQ ID NO: 12 | ACUGUGACCGAUCAAGUGGAA |
| Duplex | SEQ ID NO: 13 | CUGAUGACACUGGCUAGUUCACCTT |
| RNA-6 | SEQ ID NO: 14 | GGGACUACUGUGACCGAUCAAGUGGAA |
| Duplex | SEQ ID NO: 15 | CUGAUGACACUGGCUAGTT |
| RNA-7 | SEQ ID NO: 16 | GGGACUACUGUGACCGAUCAA |
| Single RNA | SEQ ID NO: 17 | CUGAUGACACUGGCUAGUUCACCTT |
| 8 | | |
| Single RNA | SEQ ID NO: 52 | GGGACUACUGUGACCGAUCAAGU |
| 9 | | |

*See also, FIG. 7

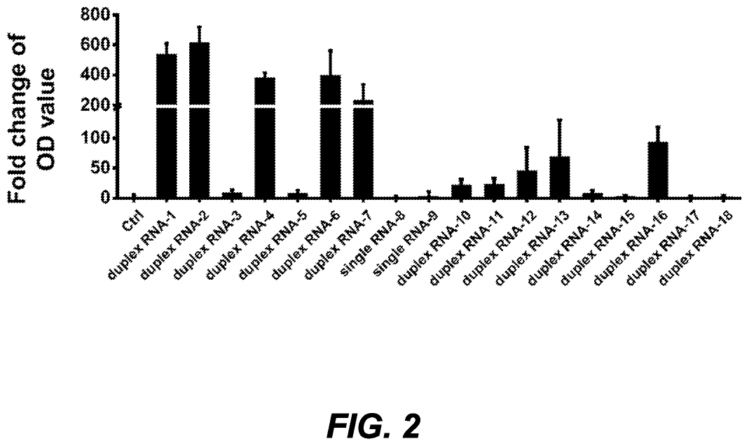

To explore this hypothesis, different versions of duplex RNAs and single-stranded RNAs were designed (Table 1, FIG. 7). It was found that keeping the common sequence in gray (duplex RNA-4) or both common sequences in gray and light gray (duplex RNA-6) while shuffling remaining sequence can induce similar levels of IFN production (Table 1, FIG. 7; FIG. 2), suggesting that the common sequence in gray is necessary for inducing IFN production while the sequence in light gray is not necessary. This is verified by duplex RNA-5, which does not have the common sequence in gray and does not induce IFN production (Table 1, FIG. 7; FIG. 2). In addition, shorter RNA sequences containing the common sequence in gray (duplex RNA-3 and duplex RNA-7) exhibit different abilities to induce IFN production (Table 1, FIG. 7; FIG. 2), suggesting that the length of duplex RNAs also affects the ability of the common sequence in gray to induce IFN production. It was also found that single stranded RNAs (single RNA-8 and single RNA-9) cannot induce IFN production (Table 1, FIG. 7; FIG. 2), and thus the duplex structure is key.

### Duplex RNAs Negatively Regulate Type I Interferon Pathway Via Modulating IRF3

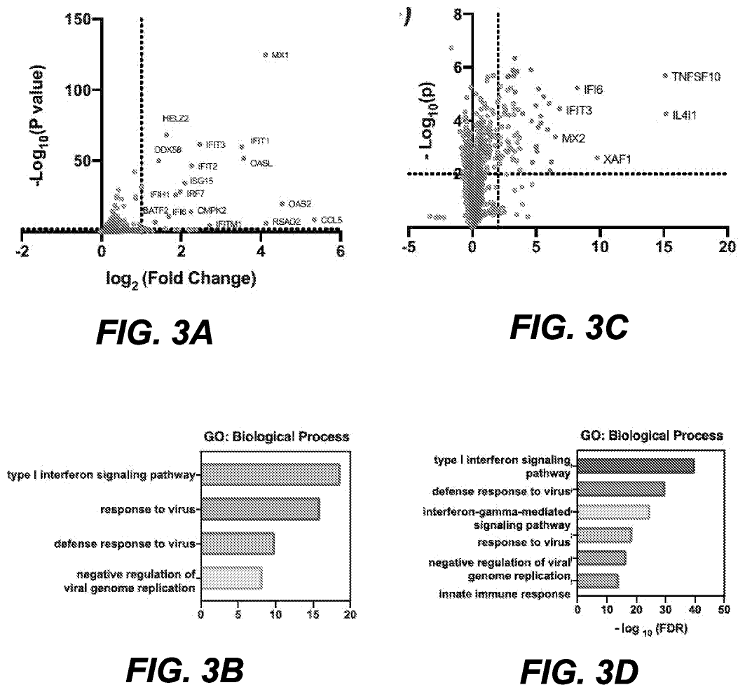

To characterize the mechanism by which these duplex RNAs reduce viral infection, RNA-seq was used to characterize transcriptome changes. Aftertreatment with duplex RNA1, 21 genes exhibited more than 2-fold increases with a threshold p value of 0.01 (FIG. 3A). Gene Ontology (GO) enrichment analysis revealed that the biological processes of these genes relate to Type I IFN signaling pathway and the defense response to viral infections (FIG. 3B). In parallel, Tandem Mass Tag (TMT) Mass Spectrometry quantification revealed upregulation of 73 proteins that have more than 4-fold increase with a threshold p value of 0.01 (FIG. 3C). GO enrichment analysis also confirmed an association between treatment of duplex RNA1 and upregulation of Type I IFN pathways (FIG. 3D). qPCR assay further validated that duplex RNA-1 mainly activates the Type I IFN pathway compared to Type II IFN pathway (Tables 2-3, see below).

TABLE 2
The effect of duplex RNA-1 on genes of IFN-α/β

| | Fold change of gene expression levels | |
|---|---|---|
| | | Duplex |
| Gene | Control | RNA-1 |
| IFI6 | 1 | 212.35 |
| IFIT2 | 1 | 1581.71 |
| IFNAl | 1 | 7.25 |
| IFNA2 | 1 | 282.76 |
| IFNA4 | 1 | 11.92 |
| IFNA5 | 1 | 85.49 |
| IFNAR1 | 1 | 1.15 |
| IFNAR2 | 1 | 1.44 |
| IFNB1 | 1 | 5665.91 |
| IRF1 | 1 | 17.88 |
| IRF2 | 1 | 5.74 |
| IRF9 | 1 | 12.48 |
| ISG15 | 1 | 380.92 |
| JAKI | 1 | 0.97 |
| PML | 1 | 13.11 |
| PRMT1 | 1 | 1.01 |
| PTPN1 | 1 | 0.86 |
| PTPN11 | 1 | 0.96 |
| PTPN6 | 1 | 1.18 |
| SOCS1 | 1 | 81.39 |
| STAT1 | 1 | 10.96 |
| STAT2 | 1 | 25.30 |
| TYK2 | 1 | 1.58 |
| USP18 | 1 | 26.83 |
| TBP | 1 | 1.04 |
| HPRT1 | 1 | 0.99 |

TABLE 3
The effect of Duplex RNA-1 on the genes of IFN-γ pathway

| | Fold change of gene expression levels | |
|---|---|---|
| Gene | Control | Duplex RNA-1 |
| AKTI | 1 | 2.04 |
| AKT2 | 1 | 2.48 |
| AKT3 | 1 | 1.80 |
| BRCA1 | 1 | 1.45 |
| CALM1 | 1 | 1.26 |
| CALM2 | 1 | 0.80 |
| CALM3 | 1 | 2.01 |
| CAMK2G | 1 | 1.21 |
| CBL | 1 | 2.56 |
| CDKN1A | 1 | 2.11 |
| CEBPB | 1 | 1.13 |
| CREBBP | 1 | 2.44 |
| EIF2AK2 | 1 | 9.97 |
| EP300 | 1 | 2.62 |
| ICAM1 | 1 | 5.08 |
| IFNG | | |
| IFNGR1 | 1 | 2.02 |
| IFNGR2 | 1 | 1.99 |
| IRF1 | 1 | 24.19 |
| JAKI | 1 | 1.58 |
| JAK2 | 1 | 7.81 |
| MAP2K1 | 1 | 1.54 |
| MAP2K6 | 1 | 2.02 |
| MAPK1 | 1 | 1.63 |
| MAPK14 | 1 | 1.73 |
| MAPK3 | 1 | 2.06 |
| MYC | 1 | 1.39 |
| PIK3CA | 1 | 1.58 |
| PIK3CB | 1 | 1.36 |
| PIK3R1 | 1 | 1.32 |
| PLCG2 | 1 | 1.73 |
| PRKCA | 1 | 1.30 |
| PRKCD | 1 | 2.70 |
| PTK2B | 1 | 4.25 |
| PTPN11 | 1 | 1.30 |
| SMAD7 | 1 | 1.24 |
| SOCS1 | 1 | 86.26 |
| SRC | 1 | 1.40 |
| STATI | 1 | 15.01 |
| TBP | 1 | 1.24 |
| HPRTI | 1 | 0.83 |

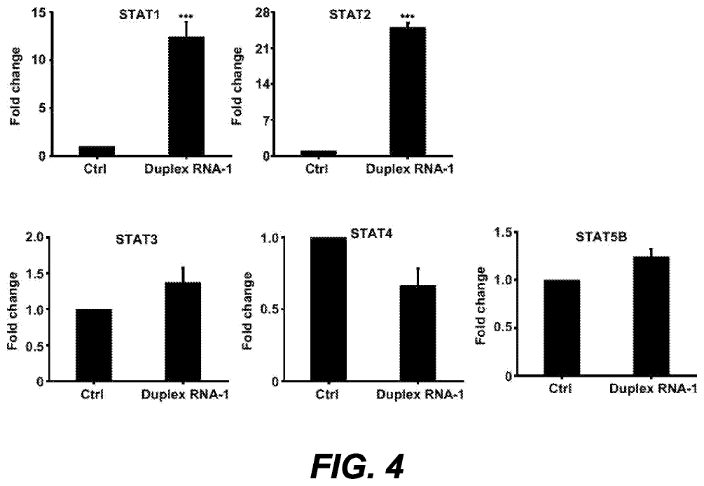

In addition, duplex RNA-1 can only increase the levels of STAT1 and STAT2 that are specific for IFN pathway (FIG. 4). These results indicate that treatment with duplex RNA-1 can specifically activate the Type I IFN pathway, which explains why treatment with duplex RNA-1 suppresses influenza infection.

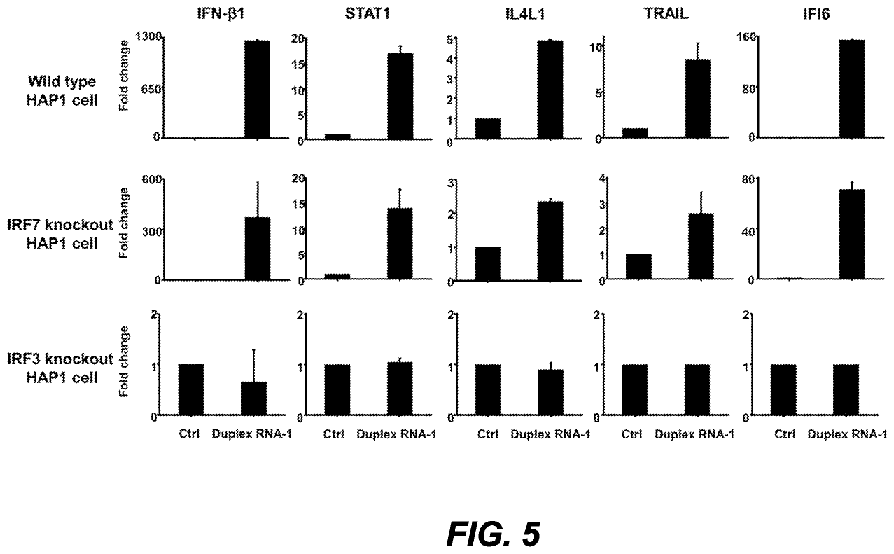

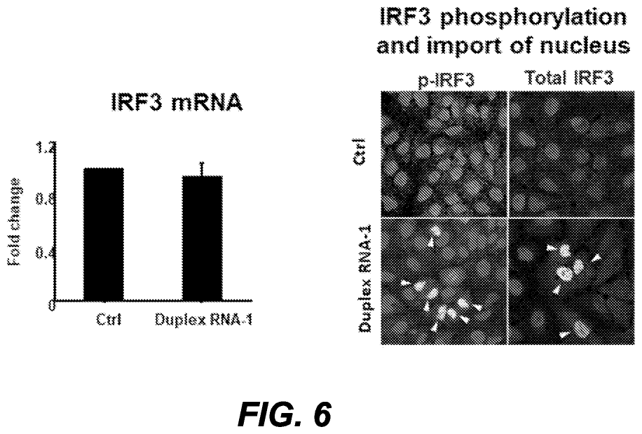

The effects of duplex RNA-1 on the Type I IFN system were further explored in wild-type, interferon regulatory factor 3 (IRF3)-knockout, and IRF7-knockout HAP1 cells. IRF3 and IRF7 are transcription factors that play vital roles in interferon-I (IFN-1) production and function in viral infection 7. Our results revealed that knock out of IRF3, but not IRF7, abolished the ability of duplex RNA1 to activate the Type I IFN pathway (FIG. 5). Taken together, our results suggest that duplex RNA1 positively regulates the Type I IFN pathway via IRF3. Further exploration indicated that duplex RNA-1 does not affect the expression level of IRF3, but it alters its phosphorylation state (FIG. 6). A similar mechanism was also observed for duplex RNA-2.

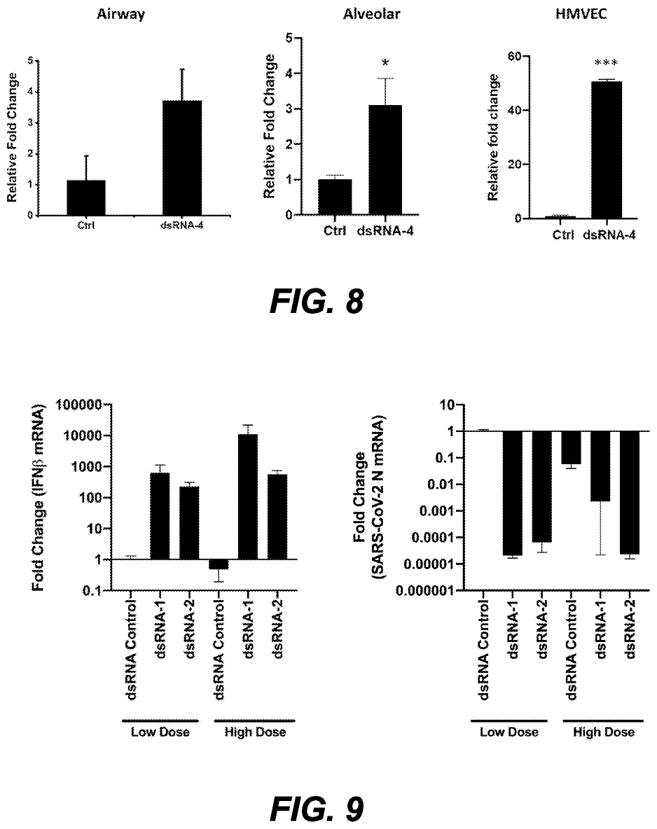

To determine whether the duplex RNAs can increase interferon production in human primary alveolar epithelium, differentiated human primary airway epithelial cells, human primary alveolar epithelial cells or human lung primary microvascular endothelial cells (HMVEC) were transfected with Negative control (NC) or dsRNA-4 using the airway chips described in Benam et al. Nature Methods (2016). Following addition of dsRNA-4, qPCR was performed 48 hours later to measure the IFN-β production. dsRNA-4 increases interferon-β production almost 4-fold compared to control airway chips (FIG. 8). Therefore, dsRNA-4 also increases interferon-β production in human primary alveolar epithelial cells on a chip, in addition to the airway and endothelial cells.

Further, data presented herein show that increased interferon-β production resulted in a marked reduction in SARS-CoV-2 N mRNA in cells infected with the SARS-CoV-2 virus (FIG. 9). The “N” in SARS-CoV-2 N mRNA refers to the gene of SARS-CoV-2 encoding the viral nucleocapsid. ACE2-expressing A549 cells were transfected with dsRNA-1 and dsRNA-2 at a low or high dose, resulting in a varied increase of interferon-β production. The transfected ACE2-A549 cells were infected with SARS-CoV-2 at an MOI 0.05 24-hours post transfection. 48-hours post infection, cells were harvested, total RNA was isolated, and qPCR was performed to detect the levels of specific genes. SARS-CoV-2 N mRNA was found to be reduced by approximately 10⁴-fold as compared to the dsRNA-control in the infected cells. Thus, these data confirm that activation of interferon-β production is effective at reducing, inhibiting or preventing viral infection, specifically a SARS-CoV-2 infection.

Given that these specific duplex RNAs can activate the Type I IFN pathway, they can be used as broad-spectrum prophylactics and therapeutics for IFN-associated diseases, including infection by a wide range of viral, bacterial, fungal, and parasitic pathogens, as well as cancers and autoimmune diseases, in addition to inhibiting influenza virus infection as shown in our proof-of-principle studies. As described above, induction of Type I IFN signaling may be particularly helpful in treating patients with COVID19 where this pathway is unusually suppressed.

To treat viral infection, IFN pathway-activating duplex RNAs can be delivered directly to the lung epithelium by aerosol, nebulizer or tracheal lavage using nanoparticle, liposome, droplet, or other formulation. Alternatively, the duplex RNAs may be delivered via intravenous, subcutaneous, intraperitoneal or intramuscular injection, with or without use of drug delivery vehicles. The administration routes and dosages of these duplex RNA molecules would need to be optimized depending on the disease being treated.

### Summary of Results:

Described herein are duplex RNAs that share common sequences, which can inhibit viral infection by inducing IFN production.

Furthermore, it was determined that IRF3 mediates the effects of the duplex RNAs on IFN expression.

As the IFN-I pathway is involved in many diseases, the duplex RNAs or related molecules containing the same key functional double stranded polynucleotide sequences represent new therapeutics for the intervention in various immune-related diseases that rely upon the IFN response, including various types of pathogenic infections caused by viruses, bacteria, fungi, or parasites, cancers, and autoimmune disorders.

### REFERENCES

1 Barber, G. N. Host defense, viruses and apoptosis. Cell Death Differ 8, 113-126, doi:10.1038/sj.cdd.4400823 (2001).
2 Blanco-Melo, D. et al. SARS-CoV-2 launches a unique transcriptional signature from in vitro, ex vivo, and in vivo systems. bioRxiv, doi:10.1101/2020.03.24.004655 (2020).
3 Benam, K. H. et al. Small airway-on-a-chip enables analysis of human lung inflammation and drug responses in vitro. Nat Methods 13, 151-157, doi:10.1038/nmeth.3697 (2016).
4 Si, L. et al. Discovery of influenza drug resistance mutations and host therapeutic targets using a human airway chip. bioRxiv, doi:10.1101/685552 (2019).
5 Si, L. et al. Human organs-on-chips as tools for repurposing approved drugs as potential influenza and COVID19 therapeutics in viral pandemics. bioRxiv, doi:10.1101/2020.04.13.039917 (2020).
6 Kim, D. H. et al. Synthetic dsRNA Dicer substrates enhance RNAi potency and efficacy. Nat Biotechnol 23, 222-226, doi: 10.1038/nbt1051 (2005).
7 Liu, S. et al. Phosphorylation of innate immune adaptor proteins MAVS, STING, and TRIF induces IRF3 activation. Science 347, aaa2630, doi:10.1126/science.aaa2630 (2015).

### Example 2: Inhibition of SARS-COV-2, HCOV-NL63, and Influenza by 5′-Monophosphate RNAs that Induce Type I Interferon

The COVID-19 crisis has clarified the need for therapeutics that can inhibit infection by the SARS-CoV-2 virus as well as other highly infectious virus variants that could cause future pandemics. Provided herein is a new class of immunostimulatory duplex RNAs containing a 5′-monophosphate that inhibit SARS-CoV-2, HCoV-NL63, and influenza virus infections by potently inducing production of type I interferon (IFN-I), and particularly IFN-β in a wide range of cells, including highly differentiated, primary, lung airway, alveolar epithelium, and microvascular endothelium grown within a microfluidic human organ-on-a-chip. These RNAs lack any sequence or structure characteristics of known immunostimulatory RNAs, and instead require a unique conserved sequence motif (sense strand: 5′-CUGA-3′ (SEQ ID NO: 1), antisense strand: 3′-GGGACU-5′) and a minimum length of 20 bases for their immunostimulatory activity. RNAs containing this motif surprisingly induce IFN-I production through activation of the RIG-I/IRF3 pathway even though they contain a 5′-monophosphate. This new class of immunostimulatory RNAs may prove useful in the future as broad-spectrum prophylactics or therapeutics for viral infections and pandemics, including COVID-19, as well as for other diseases that involve abnormal IFN-I regulation.

Coronavirus Disease 2019 (COVID-19) is a global health crisis caused by severe acute respiratory syndrome coronavirus 2 (SARS-CoV-2). Without approved drugs or vaccines to treat or prevent the disease, there has been a desperate search for new modes of therapeutic intervention. Type I and III interferons (IFN-I and IFN-III) produced by host cells when confronted by pathogens represent the first line of natural host defense against viral infection. Recognition of viral components by cellular sensors, such as Toll-like receptor 3 (TLR3), retinoic acid-inducible gene I (RIG-I), and melanoma differentiation-associated protein 5 (MDA-5), initiate a signaling cascade that induces secretion of IFN-I/-III and subsequent upregulation of hundreds of interferon-stimulated genes (ISGs), which mediate the biological and therapeutic effects of this antiviral response (1). Due to their potent and broad-spectrum effects, recombinant IFN-I and IFN-III or their synthetic inducers have been explored for treatment of viral infections, as well as autoimmune diseases and cancer (2-4). As SARS-CoV-2 has been shown to interfere with the induction of IFN-I pathway (5, 6), they are also being examined for potential therapeutic efficacy in patients with COVID-19 (4, 7-9). However, past use of recombinant IFN for treatment of related pathogenic coronaviruses, SARS-CoV and MERS-CoV, was equivocal in terms of its ability to reduce viral loads, and thus it has been suggested that there is a need to explore alternative approaches to harness this natural protective mechanism, including use of synthetic agonists (4). Moreover, the nonstructural protein 1 (nsp1) of SARS-CoV-2 was recently shown to block RIG-I-dependent innate immune responses, which otherwise facilitate clearance of the infection (10). Thus, development of immunostimulatory therapies that activate host antiviral IFN responses by counteracting inhibition of the RIG-I pathway may represent a promising strategy for combating COVID-19.

### Results

### Discovery of IFN-I Pathway-Activating Immunostimulatory RNAs

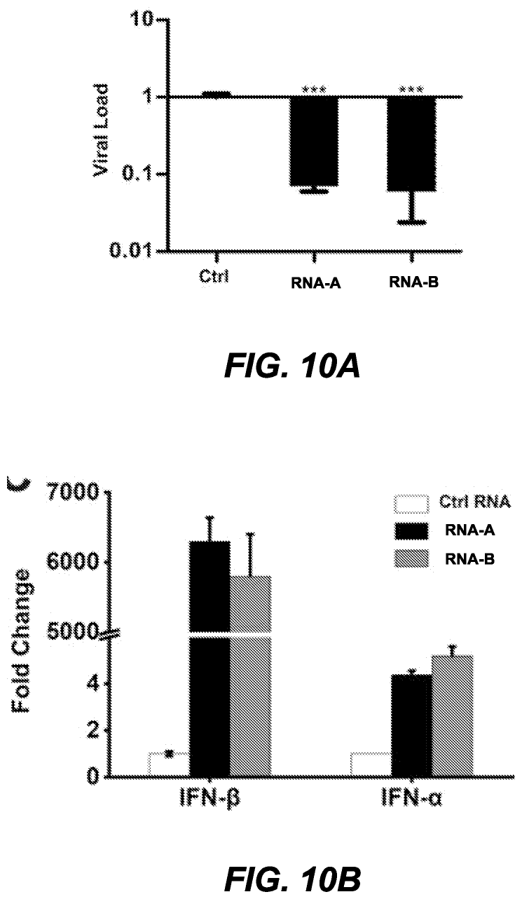

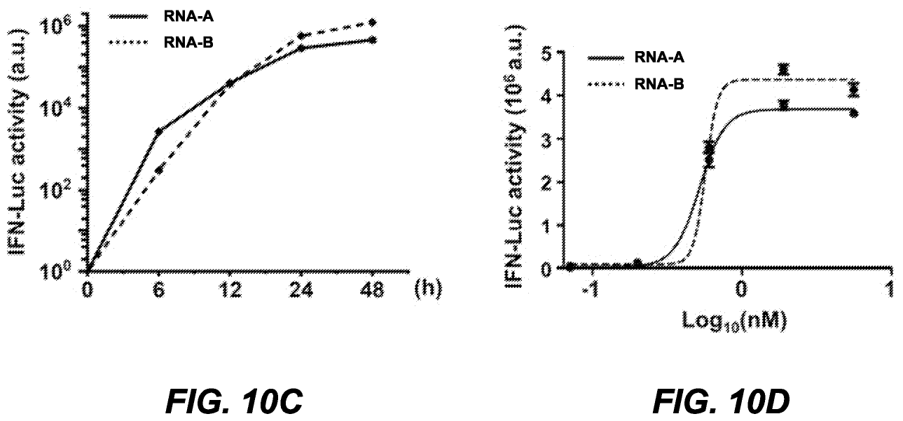

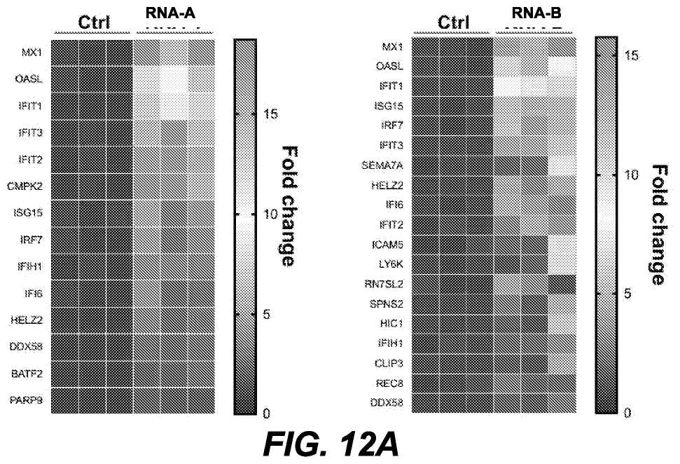

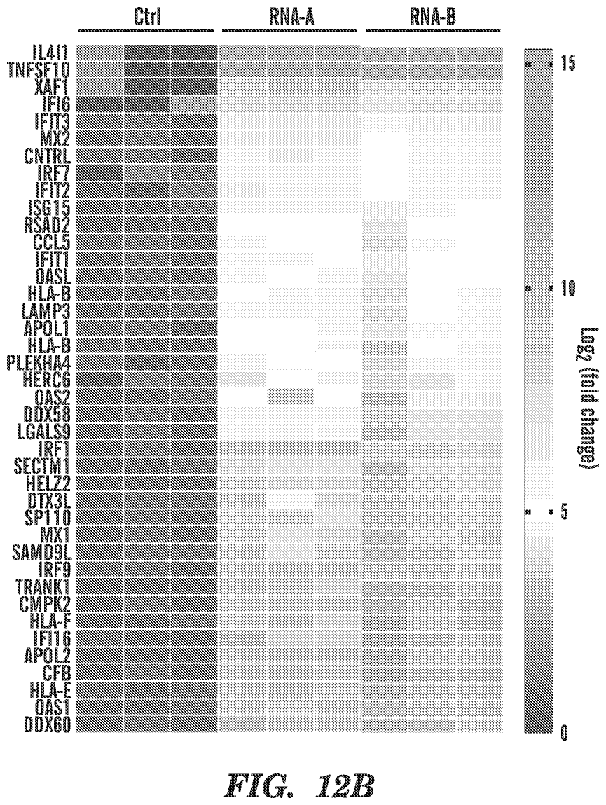

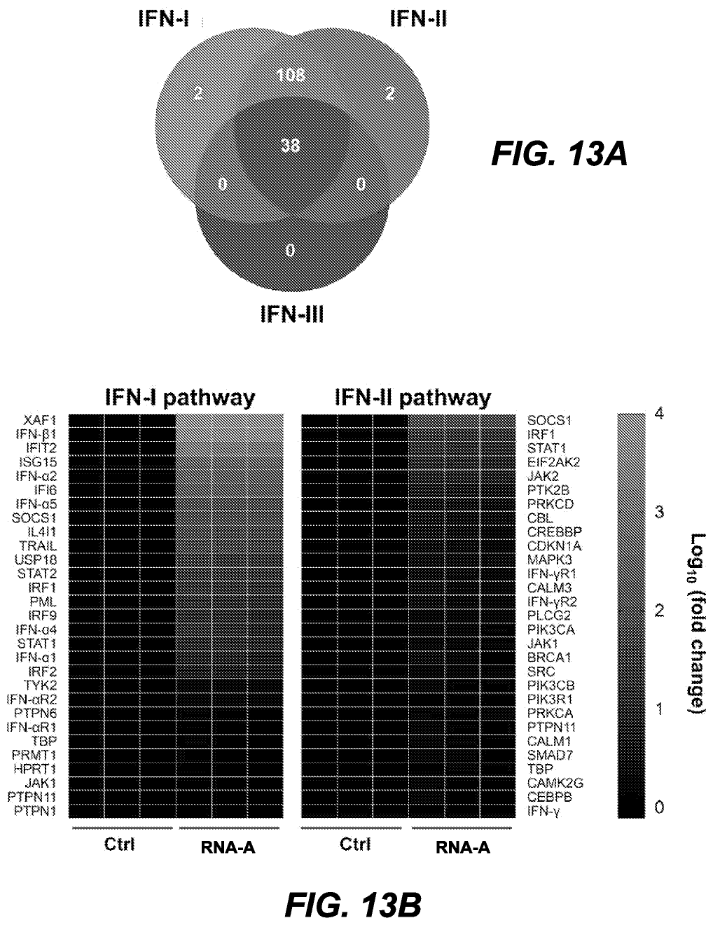

Novel RNA inducers of the IFN-I pathway that exhibit potent broad-spectrum inhibitory activity against SARS-CoV-2 as well as other viruses were discovered, serendipitously. While using >200 small interfering RNA molecules (siRNAs) to identify host genes that mediate human A549 lung epithelial cell responses to influenza A/WSN/33 (H1N1) infection, it was found that transfection of two siRNAs (RNA-A and RNA-B) inhibited H1N1 infection by more than 90% (FIG. 10A). To explore the mechanism of action of these siRNAs, which respectively targeted the long non-coding RNAs (lncRNAs) DGCR5 and LINC00261, the transcriptome and proteome of A549 cells transfected with RNA-A were profiled and RNA-B (FIG. 11A-11B) using a scrambled siRNA as a control. RNA-seq analysis showed that RNA-A upregulates the expression of 21 genes by more than 2-fold (threshold p value of 0.01) (FIG. 11A). Gene Oncology (GO) enrichment analysis revealed that these genes are involved in IFN-I signaling pathway and host defense response to viral infections including MX1, OASL, IFIT1, and ISG15 (FIG. 12A, left) In parallel, Tandem Mass Tag Mass Spectrometry (TMT Mass Spec) quantification demonstrated upregulation of 73 proteins by more than 4-fold (threshold p value of 0.01), including IL411, TNFSF10, XAF1, IF16, and IFIT3 (FIG. 12B). GO enrichment analysis of these upregulated proteins also confirmed an association between treatment of RNA-A and induction of the IFN-I pathway (FIG. 13A). Quantitative reverse transcription polymerase chain reaction (qRT-PCR) assay independently validated that RNA-A preferentially activates the IFN-I pathway relative to the Type II or III IFN pathways (FIG. 13B), with IFN-β being induced to much higher levels (>1000-fold) compared to IFN-α (FIG. 10B). Similar patterns of gene and protein expression were also observed for RNA-B (FIG. 10B and FIGS. 11-12).

Interestingly, when studies were carried out with additional siRNAs to further validate the function of the lncRNAs they target, it was found that knockdown of DGCR5 or LINC00261 by these other siRNAs did not induce IFN production. This was surprising because since the inception of RNA interference technology, short duplex (double stranded) siRNAs have been known to induce IFN-I (11, 12) and subsequent designs of these molecules, including the ones used in our study, were optimized to avoid this action and potential immunomodulatory side effects (13). siRNAs synthesized by phage polymerase that have a 5′-triphosphate end can trigger potent induction of interferon α and β (12), and siRNAs containing 9 nucleotides (5′-GUCCUUCAA-3′) at the 3′ end can induce IFN-α through TLR-7 (14), but the duplex RNAs described herein do not have either of these structures. The RNAs described herein also contained a 5′-monophosphate that is present in host RNAs, which actively suppresses activation of IFN-I via RIG-I signaling by duplex RNAs containing 5′-di- or -triphosphates (15). Thus, these data suggested that the two specific RNAs found to be potent IFN-I inducers (RNA-A and RNA-B) may represent novel immunostimulatory RNAs.

To explore this further, IFN-I production induced by the two putative immunostimulatory RNAs were evaluated using an A549-Dual™ IFN reporter cell line, which stably expresses luciferase genes driven by promoters containing IFN-stimulated response elements (16). These studies revealed that both RNA-A and RNA-B induce IFN production beginning as early as 6 hours post transfection, consistent with IFN-I being an early-response gene in innate immunity, and high levels of IFN expression were sustained for at least 24 to 48 hours (FIG. 10C). Dose-dependent induction of IFN production by these duplex RNAs was also observed over the nM range (FIG. 10D). Taken together, these results confirmed that the two siRNAs identified were indeed immunostimulatory RNAs that specifically upregulate strong IFN-I responses.

A Novel Immunostimulatory RNA Motif that is Sensed by RIG-I

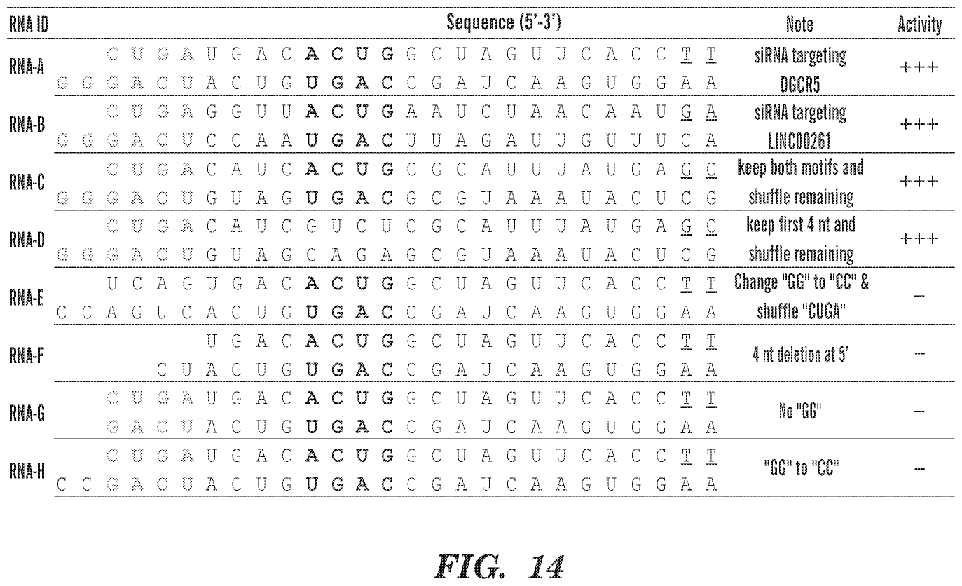

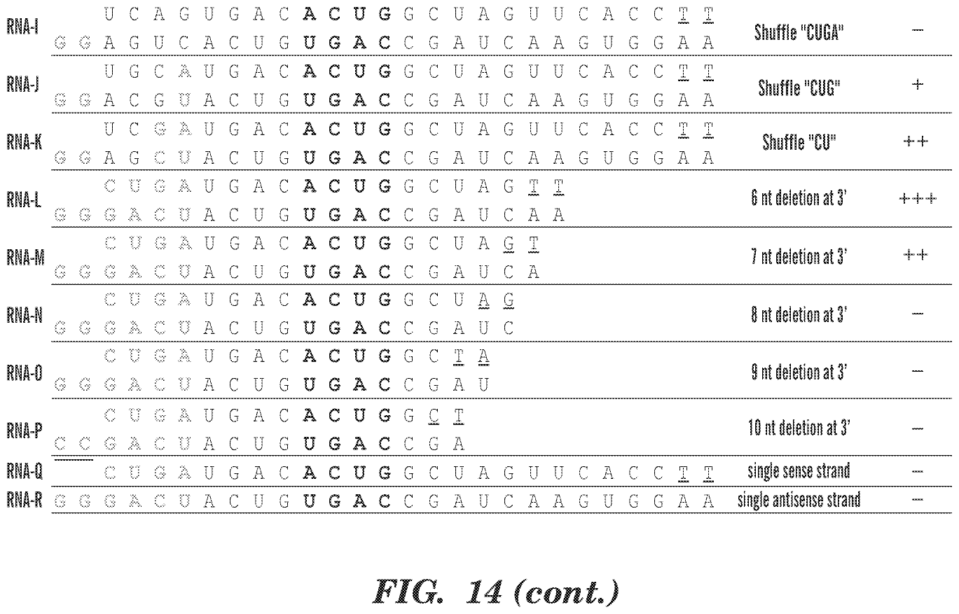

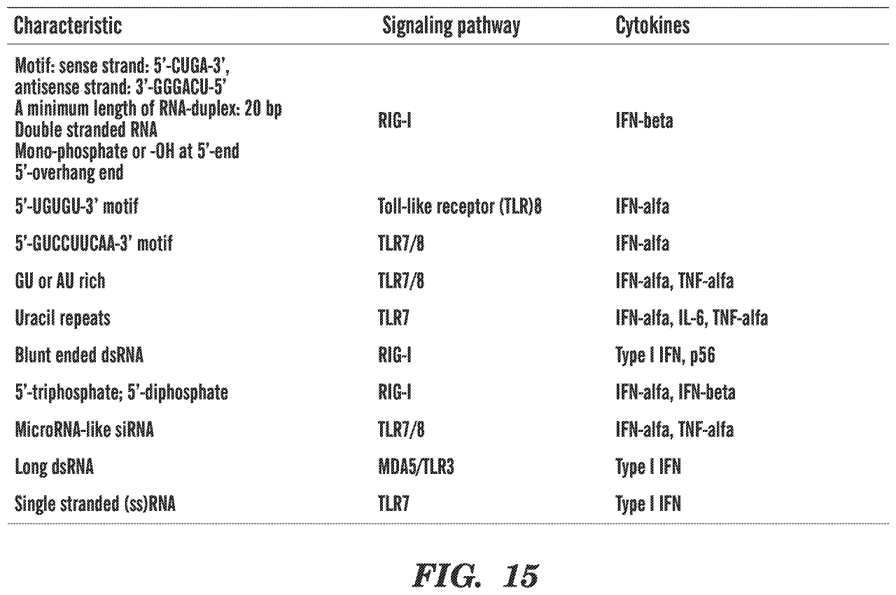

The active RNAs-1 and -2 are chemically synthesized 27mer RNA duplexes that include a 5′-monophosphate and a single, 2-base, 3′ overhang on the antisense strand (FIG. 14, Table 4 below). Their sequence and structure features do not conform to any characteristics of existing immunostimulatory RNA molecules (FIG. 15), suggesting that previously unknown elements must be responsible for this immunological activity.

TABLE 4
RNA Duplex Sequences

| | SEQ ID NOs: |
|---|---|
| | Top strand (sense) |
| RNA Duplex Name | Bottom strand (antisense) |
| RNA-A | SEQ ID NO: 13 |
| | SEQ ID NO: 14 |
| RNA-B | SEQ ID NO: 5 |
| | SEQ ID NO: 6 |
| RNA-C | SEQ ID NO: 19 |
| | SEQ ID NO: 20 |
| RNA-D | SEQ ID NO: 9 |
| | SEQ ID NO: 10 |
| RNA-E | SEQ ID NO: 21 |
| | SEQ ID NO: 22 |
| RNA-F | SEQ ID NO: 23 |
| | SEQ ID NO: 24 |
| RNA-G | SEQ ID NO: 25 |
| | SEQ ID NO: 26 |
| RNA-H | SEQ ID NO: 27 |
| | SEQ ID NO: 28 |
| RNA-I | SEQ ID NO: 29 |
| | SEQ ID NO: 30 |
| RNA-J | SEQ ID NO: 31 |
| | SEQ ID NO: 32 |
| RNA-K | SEQ ID NO: 33 |
| | SEQ ID NO: 34 |
| RNA-L | SEQ ID NO: 15 |
| | SEQ ID NO: 16 |
| RNA-M | SEQ ID NO: 35 |
| | SEQ ID NO: 36 |
| RNA-N | SEQ ID NO: 37 |
| | SEQ ID NO: 38 |
| RNA-O | SEQ ID NO: 39 |
| | SEQ ID NO: 40 |
| RNA-P | SEQ ID NO: 7 |
| | SEQ ID NO: 8 |
| RNA-Q | SEQ ID NO: 17 |
| RNA-R | SEQ ID NO: 18 |

Remarkably, even though they were designed to target different host genes, sequence alignment revealed that RNA-A and -B contained two identical motifs, with one at their 5′ ends (motif-1; sense strand: 5′-CUGA-3′ (SEQ ID NO: 1), antisense strand: 3′-GGGACU-5′) and the other in the middle region (motif-2; sense strand: 5′-ACUG-3′, antisense strand: 3′-UGAC-5′) (FIG. 14). Because both RNAs were potent inducers of IFN-β, and without being bound by a particular theory, it was contemplated that these common motifs may mediate their immunostimulatory activities.

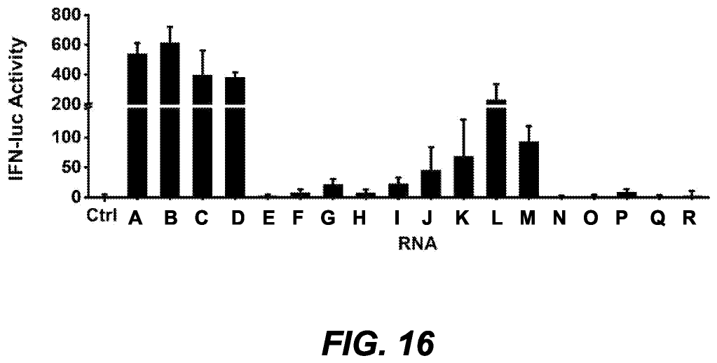

To test this, IFN-I production induced by 18 different sequence variants of RNA-A (FIG. 14) was systematically investigated using the IFN reporter expressing cell line. Maintaining motif-1 and motif-2 while replacing the remaining nucleotides with a random sequence (RNA-C vs. -A and -B) did not affect the immunostimulatory activity of the duplex RNA (FIG. 16 and FIG. 14). Further substitution of motif-2 with a random sequence also did not affect its immunostimulatory activity (RNA-D vs. -RNA-C), suggesting that motif-1 rather than motif-2 is responsible for duplex RNA-mediated IFN-I production. This was verified by reshuffling or deleting motif-1 while keeping the remaining nucleotides intact, which completely abolished the RNA's immunostimulatory activity (RNA-E and -F vs. -A).

To determine the minimal sequence in motif-1 responsible for the immunostimulatory activity, variants were generated by deleting or replacing the nucleotides of motif-1. Deletion or substitution of the two overhang bases GG at the 3′ end of the antisense strand abolished their immunostimulatory activity (RNA-G and -H vs. -A). Keeping the overhang bases GG while changing the remaining bases of motif-1 also significantly decreased (RNA-J and -K) or even completely abolished the immunostimulatory activity (RNA-I vs. -A). These data confirm that motif-1 is necessary for IFN-I induction and that its immunostimulatory efficiency is sensitive to sequence substitution or deletion. The effects of RNA length on motif-1-mediated IFN production were further evaluated by gradually trimming bases from the 3′ end of RNA-A. Removal of increasing numbers of bases resulted in a gradual decrease in immunostimulatory activity (RNA-L and -M vs. -A) with complete loss of activity when 8 bases or more were removed from the 3′ end of RNA-A (RNA-N, -O, and -P). Therefore, the minimal length of this novel form of immunostimulatory RNA required for IFN induction is 20 bases (antisense strand). In addition, neither the single sense strand nor the single antisense strand of RNA-A alone induced IFN production (RNA-Q and —R), indicating that a double stranded RNA structure is required for its immunostimulatory activity. As the chemically synthesized RNAs contain a 5′-hydroxyl group, it was also tested whether adding a 5′-monophosphate affects the interferon-inducing activity. This is important to explore because a 5′-monophosphate is present in host RNAs, which actively suppress activation of IFN-I via RIG-I signaling induced by duplex RNAs containing 5′-di- or -triphosphates. However, it was found that RNA-1 containing 5′-monophosphate induced IFN-β expression to a similar level as RNA-1 containing a 5′-hydroxyl when analyzed by qPCR (FIG. 2B), suggesting that while the 5′ monophosphate of these short duplex RNAs is not required for their effect, it does not interfere with it either.

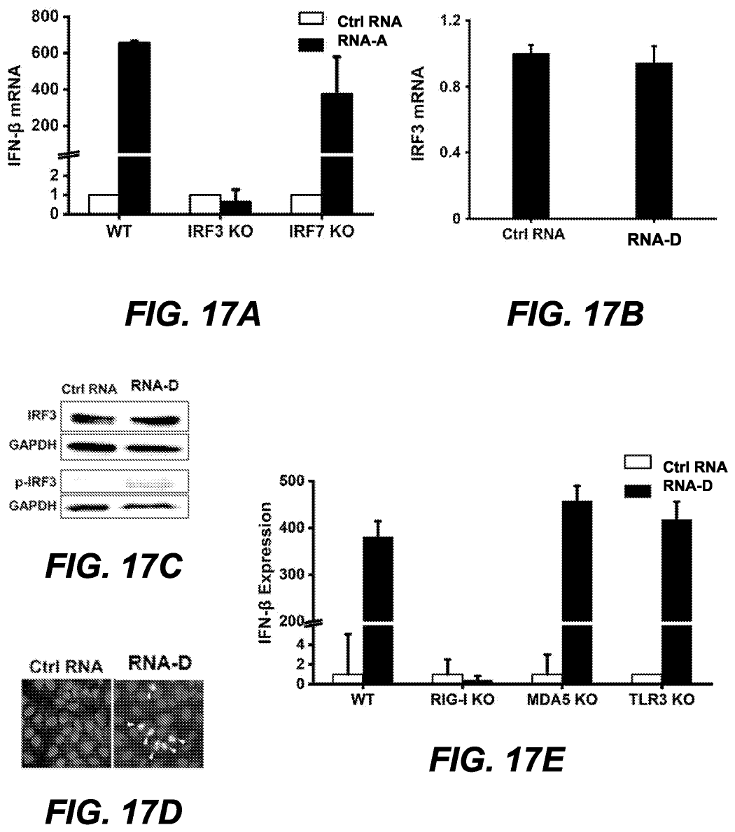

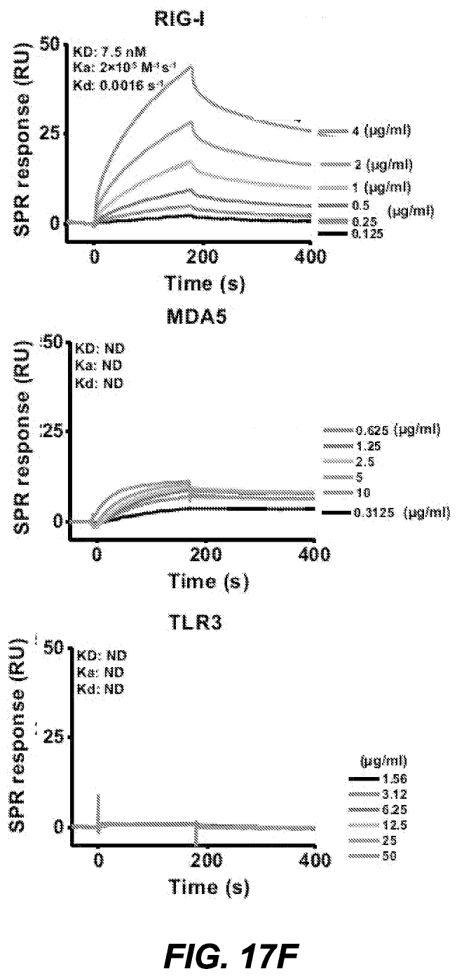

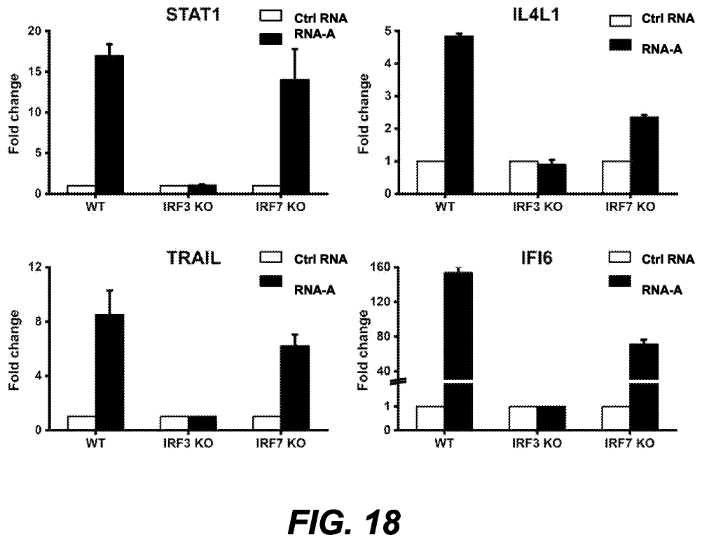

Transcription factor interferon regulatory factor 3 (IRF3) and 7 (IRF7) play vital roles in IFN-I production (17, 18). Using IRF3 knockout (KO) and IRF7 KO cells, it was found that loss of IRF3, but not IRF7, completely abolished the ability of RNA-A to induce IFN-β (FIG. 17A) and downstream ISGs, including STAT1, IL4L1, TRAIL, and IF16 (FIG. 18). IRF3 is the master and primary transcriptional activator of IFN-I and its induction of IFN-I involves a cascade of events, including IRF3 phosphorylation, dimerization, and nuclear translocation (19, 20). To alleviate potential interference from host gene knockdown by RNA-A that was developed as an siRNA, further mechanistic studies were performed using RNA-D, which also contains the active immunostimulatory motif but does not target (silence) any host genes. Although RNA-D had no effect on IRF3 mRNA or total protein levels (FIG. 17B-17C), it increased IRF3 phosphorylation (FIG. 17C), which is essential for its transcriptional activity (17) and subsequent translocation to the nucleus (FIG. 17D), where IRF3 acts as transcription factor that induces IFN-I expression (19, 20).

Short Duplex RNAs Containing the Unique Immunostimulatory Motif Bind Directly to RIG-I

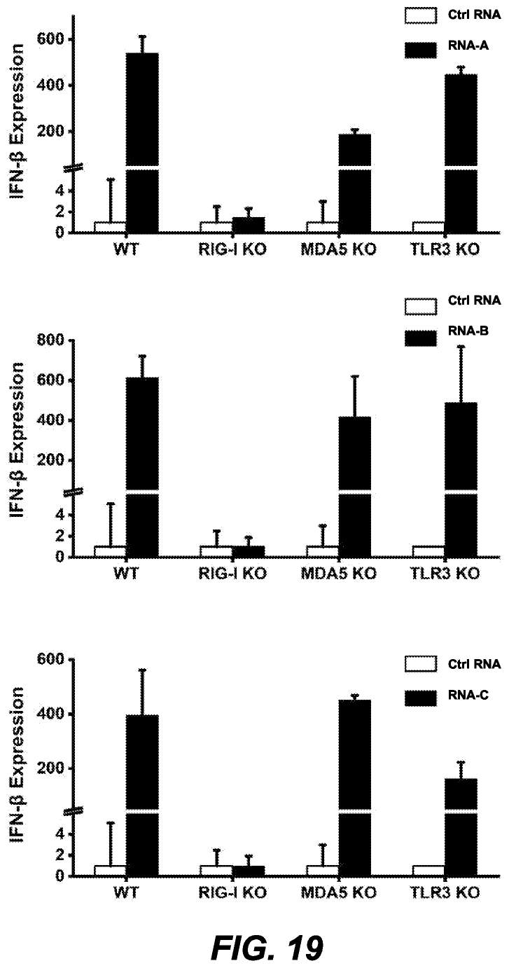

RIG-I, MDA5, and TLR3 are the main sensors upstream of IRF3 that recognize RNA (21). To investigate which of them detect our novel duplex RNAs, RNA-mediated production of IFN-I was quantitated in RIG-I, MDA5, or TLR3 KO cells. Knockout of RIG-I completely suppressed the ability of RNA-D (FIG. 17E) as well as RNA-A, -B, and -C (FIG. 19) to induce IFN-I, whereas loss of MDA5 or TLR3 had no effect on RNA-mediated IFN-I production (FIG. 17E and FIG. 19). Importantly, surface plasmon resonance (SPR) analysis revealed that RNA-1 interacts directly with the RIG-I cellular RNA sensor, rather than MDA5 or TLR3 (FIG. 3F). Thus, small duplex RNAs containing the novel immunostimulatory motif specifically use the RIG-I/IRF3 pathway to stimulate IFN-I production even though they contain a 5′-monophosphate that has been previously suggested to antagonize, rather than stimulate, RIG-I-dependent activation of IFN production (15).

Finally, these new immunostimulatory RNAs also exhibited much more (>2,000-fold) potent induction of IFN-β production when compared to either the commonly used pathogen recognition receptor (PRR) agonist, Poly (I:C), or a duplex RNA containing a 5′-triphosphosphate (FIG. S6A) that is known to be an activating RIG-I ligand. More importantly, RNA-seq analysis revealed that in contrast to Poly (I:C), these new immunostimulatory RNAs do not induce expression of a broad range of inflammation-associated genes (FIG. S6B). This is of great clinical relevance because while Poly (I:C) can induce IFN responses, it is limited in its potential use in patients due to complicating toxicities associated with induction of more generalized inflammation responses

Broad Spectrum Inhibition of Multiple Coronaviruses and Influenza a Viruses

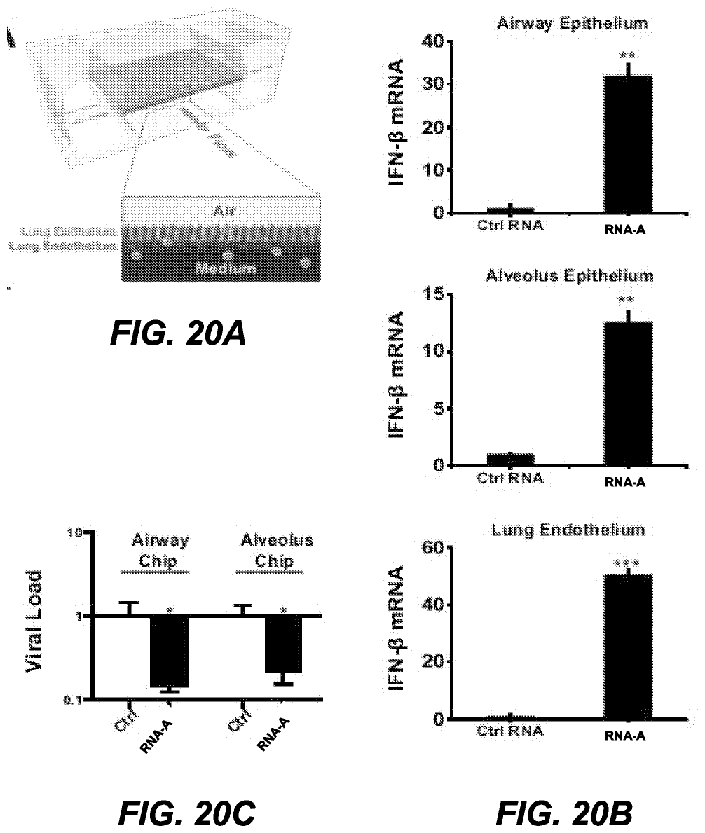

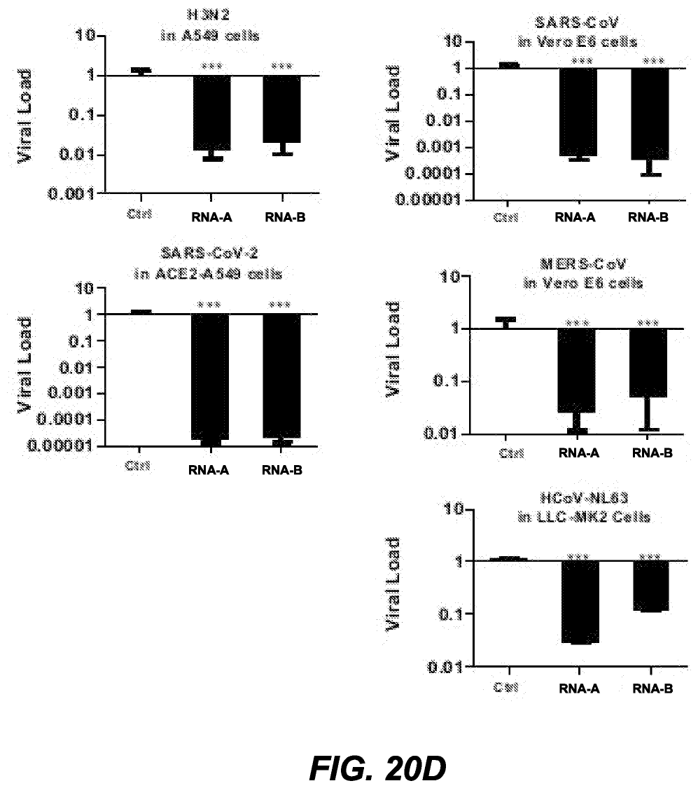

To explore the potential physiological and clinical relevance of these novel RNAs that demonstrated immunostimulatory activities in established cell lines, it was investigated whether they can trigger IFN-I responses in human Lung Airway and Alveolus Chip microfluidic culture devices lined by human primary lung epithelium grown under an air-liquid interface in close apposition to a primary pulmonary microvascular endothelium cultured under dynamic fluid flow, which have been demonstrated to faithfully recapitulate human organ-level lung physiology and pathophysiology (22-24). A 4- to 12-fold increase in IFN-β expression was observed compared to a scrambled duplex RNA control when RNA-A was transfected into human airway and alveolus epithelial cells through the air channels of the human Lung Chips (FIG. 20A). In addition, treatment with RNA-A induced robust (>40-fold) IFN-β expression in human primary lung endothelium on-chip (FIG. 20A) when it was introduced into the vascular channel of the chip.

Given the initial finding that RNA-A and -B inhibit infection by H1N1 (FIG. 1A) along with the known antiviral functions of IFN-I (25), the generality of these effects was next explored. First, the potential of these IFN-I inducing RNAs to block infection by influenza A/HK/8/68 (H3N2) virus was examined. Cells were transfected with RNAs one day prior to infection, and then with the advent of the COVID-19 pandemic, this work was extended by carrying out similar studies with SARS-CoV-2 and a related coronavirus, HCoV-NL63. Analysis with qPCR for viral mRNA revealed that treatment with the immunostimulatory RNAs significantly suppressed infections by influenza H3N2 virus in A549 cells (>95% inhibition) and in the human Lung Alveolus Chip lined by primary alveolar epithelium interfaced with pulmonary microvascular endothelium (˜ 80% inhibition) (FIG. 20B), as it did with influenza H1N1 virus in A549 cells (FIG. 10A).

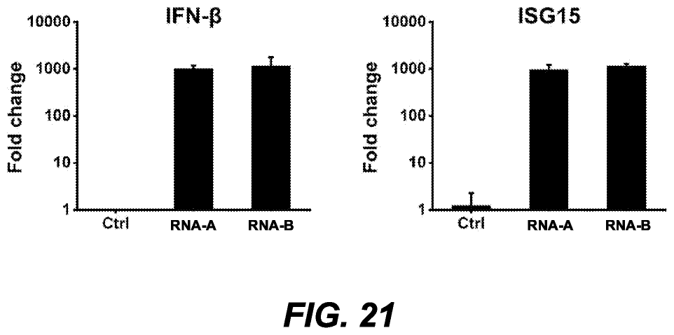

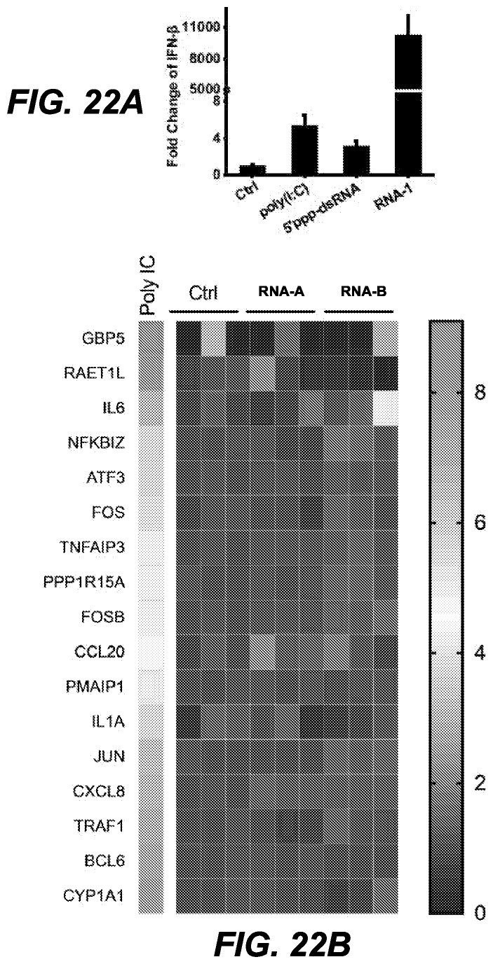

These same duplex RNAs inhibited HCoV-NL63 in LLC-MK2 cells by >90% (FIG. 20B), and impressively, they were even more potent inhibitors of SARS-CoV-2 infection, reducing viral load in ACE2 receptor-overexpressing A549 cells by over 10,000-fold (>99.99%) (FIG. 20B and FIG. 21), which is consistent with the observation that SARS-CoV-2 regulates IFN-I signaling differently and fails to induce its expression relative to these other viruses (6). Importantly, RNA-seq analysis revealed that in contrast to the commonly used pathogen recognition receptors (PRRs) agonist, poly (I:C), these immunostimulatory RNAs did not induce expression of a broad range of inflammation-relevant genes (FIG. 22). This is important because while poly (I:C) can induce IFN responses, it is limited in its potential clinical use due to complicating toxicities associated with induction of more generalized inflammation responses (26, 27).

Given the potent inhibitory activity against SARS-CoV-2 observed in vitro, RNA-1 was then evaluated in a hamster COVID-19 model. RNA-1 was dissolved in phosphate buffered saline (PBS) and administered intranasally one day before the animals were infected intranasally with SARS-CoV-2 virus (10² PFU), on the day of infection, and one day post-infection. When the SARS-CoV-2 viral N transcript was measured in the lungs of these hamsters on the second day after the viral challenge, it was found that prophylaxis with RNA-1 effectively prevented infection as it resulted in a significant (p=0.030) reduction in viral load whether measured by RT-qPCR or by quantifying viral titers using a plaque assay (p=0.032) (FIG. 5A). In addition, similar inhibition of viral infection was measured when RNA-1 was administered in a therapeutic mode by introducing it intranasally in vehicle daily for two days, beginning one day after viral infection (103 PFU), and then analyzing lungs by RT-PCR (FIG. 5B). Most importantly, histological analysis of these lungs revealed that the reduction in viral load produced by treatment with RNA-1 beginning one day after infection resulted in a major decrease in immune cell infiltration into the alveolar air spaces, which were completely obliterated and filled with cells and exudate in control infected lungs treated with vehicle alone (FIG. 5C).

### Discussion

In this study, potent stimulation of IFN-I signaling was observed, with particularly efficient induction of IFN-β relative to IFN-α, by a new class of short overhanging duplex RNAs that contain a 5′-monophosphate and a unique sequence motif in a broad spectrum of human cells. This is in contrast to previously described immunostimulatory RNAs that contain 5′-di or -triphosphates and mainly induce IFN-α or other inflammatory cytokines (28). By systematically investigating the effects of sequence and length of these RNAs on IFN-I induction, it was determined that these duplex RNAs require a minimal length of 20 bases, in addition to a conserved overhanging immunostimulatory motif (sense strand: 5′-CUGA-3′ (SEQ ID NO: 1), antisense strand: 3′-GGGACU-5′) and a 5′-monophosphate terminus to exhibit their immunostimulatory activity. Mechanistic exploration revealed that these novel immunostimulatory RNAs specifically activated RIG-I/IRF3 pathway, even though duplex RNAs with 5′-monophosphate have been previously shown to antagonize IFN signaling by RNAs with 5′-di or -triphosphates (15, 29). In addition, the RNA-mediated IFN-I production that was observed resulted in significant inhibition of infections by multiple human respiratory viruses, including influenza viruses H1N1 and H3N2, as well as coronaviruses HCoV-NL63 and SARS-CoV-2. Notably, these novel immunostimulatory RNAs reduced SARS-CoV-2 viral load by more than 10,000-fold. These findings show that these IFN-I-inducing immunostimulatory RNAs can offer a novel prophylactic or therapeutic strategy for the current COVID-19 pandemic, in addition to offering a potential broad-spectrum prophylaxis against a wide range of respiratory viruses that might emerge in the future.

Based on the overlapping sequence of two RNAs with potent IFN-β-inducing activity, a conserved overhanging immunostimulatory motif was identified to contain a sense strand 5′-CUGA-3′ (SEQ ID NO: 1) and antisense strand 3′-GGGACU-5′ with 5′-monophosphate. No immunostimulatory activity was observed for RNAs containing this motif in the middle region or at the 3′ end, suggesting that the 5′-terminal location is required for immunostimulation. This finding is consistent with studies that show RIG-I recognizes the 5′ ends of duplex RNAs (15); however, as our immunostimulatory RNAs contain a 5′-monophosphate with overhang and exhibit sequence-dependent activation of RIG-I, they do not belong to any previously known category of immunostimulatory RNA (FIG. 15), and hence, represent a new class of immunostimulatory RNAs.

The findings demonstrated above also led to the identification of a new form of cellular recognition of RNAs by cytoplasmic RNA sensors. At least four signaling pathways have been found to recognize immunostimulatory RNA molecules and induce the production of IFN-I and pro-inflammatory cytokines, including RIG-I, MDA5, TLR3, and TLR7/8 (FIG. 15). MDA5 recognizes long RNA molecules (˜0.5-7 kb in length) (30); TLR3 detects duplex RNA molecules in the endosome that are at least 40-50 bp in length (31); TLR7 and TLR8 detect GU-rich short single strand RNAs as well as small human-made molecules, such as nucleoside analogs and imidazoquinolines (32). RIG-I is a central component of the mammalian innate immune system, which detects pathogen-associated RNA molecules and inducing rapid antiviral immune responses. Previous studies indicated that RIG-I recognizes long dsRNAs (300-1,000 bp in length), RNase L-generated small self-RNAs, or short, blunt, duplex RNAs with a 5′-di- or tri-phosphate (28, 33-39). As described above, RIG-I is thought to be antagonized by RNAs containing 5′-monophosphate (15), and a separate study showed that almost any type of 5′ or 3′ overhang can prevent RIG-I binding and eliminate signaling (33). In contrast, potent sequence-dependent activation of RIG-I by short overhanging duplex RNAs with 5′-monophosphate was observed, which represents an entirely novel form of RNA recognition by RIG-I.

While immune stimulation by siRNAs is undesired in some gene silencing applications, it can be beneficial in others, such as treatment of viral infections or cancer. This raises the possibility that siRNAs with both RNAi and immunostimulatory activities may be designed to provide even greater potency. The motif identified in our study is well suited for this purpose since it is located at the 5′ end of the RNA and thus, can be coupled with sequences that target viral mRNA or other infection-associated host genes without compromising RNAi activity. The IFN response constitutes the major first line of defense against viruses, and these infectious pathogens, including SARS-CoV-2, have evolved various strategies to suppress this response (5, 6). In particular, transcriptomic analyses in both human cultured cells infected with SARS-CoV-2 and COVID-19 patients revealed that SARS-CoV-2 infection produces a unique inflammatory response with very low IFN-I, IFN-III, and associated ISG responses, while stimulating chemokine and pro-inflammatory cytokine production (5, 6), and this imbalance could contribute to the increased morbidity and mortality seen in late stage COVID-19 patients. Without approved antiviral therapeutics or vaccines to this emerging respiratory virus, type I and type III IFNs are therefore being evaluated for their efficacy in preclinical models and clinical trials (4, 8, 9, 40). Pretreatment with IFN has been shown to drastically reduces viral titers, suggesting that induction of IFN-I responses may represent a potentially effective approach for prophylaxis or early treatment of SARS-CoV-2 infections (41, 42). Triple combination of IFN-3, lopinavir, ritonavir, and ribavirin also has been recently reported to shorten the duration of viral shedding and hospital stay in patients with mild to moderate COVID-19 (7). However, treatment with IFN-β commonly requires systemic administration through injection, and thus the levels of therapeutic delivered may be limited by systemic toxicities making this difficult to be used as a prophylactic therapy.

Consistent with these observations, the results provided herein showed that pretreatment with IFN-I-inducing RNAs resulted in a dramatic decrease in infection by SARS-CoV-2, as well as HCoV-NL63 and influenza viruses. Importantly, our immunostimulatory RNAs specifically activate RIG-I/IFN-I pathway but are not recognized by other cellular RNA sensors, such as MDA5 or TLR3. This is interesting because recent studies show that SARS-CoV-2 inhibits RIG-I signaling and clearance of infection via expression of nsp1 (10), and thus our results demonstrate that these novel duplex RNAs can overcome this inhibition, at least in human lung epithelial and endothelial cells cultured maintained in Organ Chip cultures that has been previously shown to recapitulate human lung physiology and pathophysiology (43, 44). In addition, this provides a clear advantage over other immunostimulatory RNAs with regards to intrinsic toxicity. For example, the commonly used PRR agonist poly (I:C) activates multiple signaling pathways, including RIG-I, MDA5, and TLR3 (45-47), and triggers production of multiple proinflammatory cytokines and chemokines, such as TNF-α, IL-1, IL-6, and IL-8 (48), whereas the novel immunostimulatory RNAs described here do not. In addition, the fact that the RNAs described herein specifically induce IFN-I, but not IFN-III, makes them safer for clinical use against endemic viruses as IFN-III can disrupt the lung epithelial barrier upon viral recognition (40).

The finding that these novel immunostimulatory RNAs can induce IFN-I in highly differentiated, primary human lung epithelial and endothelial cells in microfluidic Organ Chips that recapitulate human pathophysiology provides additional support for their clinical use as prophylactics or therapeutics for COVID-19 or other future viral pandemics. To explore these clinical applications, the efficiency of RNA delivery can be optimized; however, the concept of an intranasal or inhaled RNA formulation (similar to an asthma inhaler) that can raise endogenous IFN-β levels many fold locally in the respiratory tract for prevention of infectious spread in the setting of a viral pandemic, such as COVID-19, is an exciting one.

### REFERENCES

1) J. W. Schoggins, Interferon-Stimulated Genes: What Do They All Do? Annu Rev Virol doi: 10.1146/annurev-virology-092818-015756. (2019).
2) E. C. Borden et al., Interferons at age 50: past, current and future impact on biomedicine. Nat Rev Drug Discov 6, 975-990 (2007).
3) A. Watson et al., Dynamics of IFN-beta Responses During Respiratory Viral Infection: Insights for Therapeutic Strategies. Am J Respir Crit Care Med. doi:10.1164/rccm.201901-0214OC. (2019).
4) A. Park, A. Iwasaki, Type I and Type III Interferons—Induction, Signaling, Evasion, and Application to Combat COVID-19. Cell Host Microbe 27, 870-878 (2020).
5) J. Hadjadj et al., Impaired type I interferon activity and inflammatory responses in severe COVID-19 patients. Science doi:10.1126/science.abc6027. (2020).
6) D. Blanco-Melo et al., Imbalanced Host Response to SARS-CoV-2 Drives Development of COVID-19. Cell 181, 1036-1045 e1039 (2020).
7) I. F. Hung et al., Triple combination of interferon beta-1b, lopinavir-ritonavir, and ribavirin in the treatment of patients admitted to hospital with COVID-19: an open-label, randomised, phase 2 trial. Lancet 395, 1695-1704 (2020).
8) M. Wadman, Can interferons stop COVID-19 before it takes hold? Science 369, 125-126 (2020).
9) M. Wadman, Can boosting interferons, the body's frontline virus fighters, beat COVID-19?Science doi:10.1126/science.abd7137. (2020).
10) M. T. e. al., Structural basis for translational shutdown and immune evasion by the Nsp1 protein of SARS-CoV-2. Science doi: 10.1126/science.abc8665. (2020).
11) C. A. Sledz, M. Holko, M. J. de Veer, R. H. Silverman, B. R. Williams, Activation of the interferon system by short-interfering RNAs. Nat Cell Biol 5, 834-839 (2003).
12) D. H. Kim et al., Interferon induction by siRNAs and ssRNAs synthesized by phage polymerase. Nat Biotechnol 22, 321-325 (2004).
13) D. H. Kim et al., Synthetic dsRNA Dicer substrates enhance RNAi potency and efficacy. Nat Biotechnol 23, 222-226 (2005).
14) V. Hornung et al., Sequence-specific potent induction of IFN-alpha by short interfering RNA in plasmacytoid dendritic cells through TLR7. Nat Med 11, 263270 (2005).
15) X. Ren, M. M. Linehan, A. Iwasaki, A. M. Pyle, RIG-I Selectively Discriminates against 5′-Monophosphate RNA. Cell Rep 26, 2019-2027 e2014 (2019).
16) J. Tissari, J. Siren, S. Men, I. Julkunen, S. Matikainen, IFN-alpha enhances TLR3-mediated antiviral cytokine expression in human endothelial and epithelial cells by up-regulating TLR3 expression. Journal of immunology 174, 4289-4294 (2005).
17) S. Liu et al., Phosphorylation of innate immune adaptor proteins MAVS, STING, and TRIF induces IRF3 activation. Science 347, aaa2630 (2015).
18) P. Wang, J. Xu, Y. Wang, X. Cao, An interferon-independent lncRNA promotes viral replication by modulating cellular metabolism. Science 358, 1051-1055 (2017).
19) Y. Zhou et al., Interferon-inducible cytoplasmic lncLrrc55-AS promotes antiviral innate responses by strengthening IRF3 phosphorylation. Cell Res 29, 641-654 (2019).
20) K. A. Fitzgerald et al., IKKepsilon and TBK1 are essential components of the IRF3 signaling pathway. Nat Immunol 4, 491-496 (2003).
21) K. T. Chow, M. Gale, Jr., Y. M. Loo, RIG-I and Other RNA Sensors in Antiviral Immunity. Annu Rev Immunol 36, 667-694 (2018).
22) K. H. Benam et al., Small airway-on-a-chip enables analysis of human lung inflammation and drug responses in vitro. Nat Methods 13, 151-157 (2016).
23) L. Si et al., Human organs-on-chips as tools for repurposing approved drugs as potential influenza and COVID19 therapeutics in viral pandemics. bioRxiv doi:10.1101/2020.04.13.039917. (2020).
24) L. Si et al., Discovery of influenza drug resistance mutations and host therapeutic targets using a human airway chip. bioRxiv doi:10.1101/685552. (2019).
25) E. V. Mesev, R. A. LeDesma, A. Ploss, Decoding type I and III interferon signalling during viral infection. Nat Microbiol 4, 914-924 (2019).
26) N. C. Stowell et al., Long-term activation of TLR3 by poly(I:C) induces inflammation and impairs lung function in mice. Respir Res 10, 43 (2009).
27) A. R. Lever et al., Comprehensive evaluation of poly(I:C) induced inflammatory response in an airway epithelial model. Physiol Rep 3, (2015).
28) Z. Meng, M. Lu, RNA Interference-Induced Innate Immunity, Off-Target Effect, or Immune Adjuvant? Front Immunol 8, 331 (2017).
29) D. Goubau et al., Antiviral immunity via RIG-I-mediated recognition of RNA bearing 5′-diphosphates. Nature 514, 372-375 (2014).
30) A. Peisley et al., Kinetic mechanism for viral dsRNA length discrimination by MDA5 filaments. Proc Natl Acad Sci USA 109, E3340-3349 (2012).
31) L. Liu et al., Structural basis of toll-like receptor 3 signaling with double-stranded RNA. Science 320, 379-381 (2008).
32) O. Takeuchi, S. Akira, Pattern recognition receptors and inflammation. Cell 140, 805-820 (2010).
33) X. Ren, M. M. Linehan, A. Iwasaki, A. M. Pyle, RIG-I Recognition of RNA Targets: The Influence of Terminal Base Pair Sequence and Overhangs on Affinity and Signaling. Cell Rep 29, 3807-3815 e3803 (2019).
34) V. Hornung et al., 5′-Triphosphate RNA is the ligand for RIG-I. Science 314, 994997 (2006).
35) A. Pichlmair et al., RIG-I-mediated antiviral responses to single-stranded RNA bearing 5′-phosphates. Science 314, 997-1001 (2006).
36) M. Schlee et al., Recognition of 5′ triphosphate by RIG-I helicase requires short blunt double-stranded RNA as contained in panhandle of negative-strand virus. Immunity 31, 25-34 (2009).
37) A. Schmidt et al., 5′-triphosphate RNA requires base-paired structures to activate antiviral signaling via RIG-I. Proc Natl Acad Sci USA 106, 12067-12072 (2009).
38) A. Kohlway, D. Luo, D. C. Rawling, S. C. Ding, A. M. Pyle, Defining the functional determinants for RNA surveillance by RIG-I. EMBO Rep 14, 772-779 (2013).
39) J. Zheng et al., High-resolution HDX-MS reveals distinct mechanisms of RNA recognition and activation by RIG-I and MDA5. Nucleic Acids Res 43, 1216-1230 (2015).
40) A. Broggi et al., Type III interferons disrupt the lung epithelial barrier upon viral recognition. Science doi: 10.1126/science.abc3545. (2020).
41) K. G. Lokugamage, Hage, A., Schindewolf, C., Rajsbaum, R., and Menachery, V. D., SARS-CoV-2 is sensitive to type I interferon pretreatment. bioRxiv doi:2020.03.07.982264., (2020).
42) E. Mantlo, N. Bukreyeva, J. Maruyama, S. Paessler, C. Huang, Antiviral activities of type I interferons to SARS-CoV-2 infection. Antiviral Res 179, 104811 (2020).
43) A. Jain et al., Primary Human Lung Alveolus-on-a-chip Model of Intravascular Thrombosis for Assessment of Therapeutics. Clin Pharmacol Ther 103, 332-340 (2018).
44) D. Huh et al., A human disease model of drug toxicity-induced pulmonary edema in a lung-on-a-chip microdevice. Sci Transl Med 4, 159ra147 (2012).
45) L. Alexopoulou, A. C. Holt, R. Medzhitov, R. A. Flavell, Recognition of double-stranded RNA and activation of NF-kappaB by Toll-like receptor 3. Nature 413, 732-738 (2001).
46) T. Kawai, S. Akira, Toll-like receptor and RIG-I-like receptor signaling. Ann N Y Acad Sci 1143, 1-20 (2008).
47) H. Kato et al., Differential roles of MDA5 and RIG-I helicases in the recognition of RNA viruses. Nature 441, 101-105 (2006).
48) J. M. Burke, S. L. Moon, T. Matheny, R. Parker, RNase L Reprograms Translation by Widespread mRNA Turnover Escaped by Antiviral mRNAs. Mol Cell 75, 1203-1217 e1205 (2019).

### Example 3: Materials and Methods

Cell Culture

A549 cells (ATCC CCL-185), A549-Dual™ cells (InvivoGen), RIG-I KO A549-Dual™ cells (InvivoGen), MDA5 KO A549-Dual™ cells (InvivoGen), TLR3 KO A549 cells (Abcam), MDCK cells (ATCC CRL-2936), and LLC-MK2 cells (ATCC CCL-7.1) were cultured in Dulbecco's modified Eagle's medium (DMEM) (Life Technologies) supplemented with 10% fetal bovine serum (FBS) (Life Technologies) and penicillin-streptomycin (Life Technologies). HAP1 cells, IRF3 KO HAP1 cells, and IRF7 KO HAP1 cells were purchased from Horizon Discovery Ltd and cultured in Iscove's Modified Dulbecco's Medium (IMDM) (Gibco) supplemented with 10% fetal bovine serum (FBS) (Life Technologies) and penicillin-streptomycin (Life Technologies). All cells were maintained at 37° C. and 5% Co₂ in a humidified incubator. All cell lines used in this study were free of mycoplasma, as confirmed by the LookOut Mycoplasma PCR Detection Kit (Sigma). Cell lines were authenticated by the ATCC, InvivoGen, Abcam, or Horizon Discovery Ltd. Primary human lung airway epithelial basal stem cells (Lonza, USA) were expanded in 75 cm² tissue culture flasks using airway epithelial cell growth medium (Promocell, Germany) until 60-70% confluent. Primary human alveolar epithelial cells (Cell Biologics, H-6053) were cultured using alveolar epithelial growth medium (Cell Biologics, H6621). Primary human pulmonary microvascular endothelial cells (Lonza, CC-2527, P5) were expanded in 75 cm² tissue culture flasks using human endothelial cell growth medium (Lonza, CC-3202) until 70-80% confluent.

Viruses

Viruses used in this study include SARS coronavirus-2 (SARS-CoV-2), human coronavirus HCoV-NL63, influenza A/WSN/33 (H1N1), and influenza A/Hong Kong/8/68 (H3N2). SARS-CoV-2 isolate USA-WA1/2020 (NR-52281) was deposited by the Center for Disease Control and Prevention, obtained through BEI Resources, NIAID, NIH, and propagated as described previously (Blanco-Melo et al., 2020). HCoV-NL63 was obtained from the ATCC and expanded in LLC-MK2 cells. Influenza A/WSN/33 (H1N1) was generated using reverse genetics technique and influenza A/Hong Kong/8/68 (H3N2) was obtained from the ATCC. Both influenza virus strains were expanded in MDCK cells. HCoV-NL63 was titrated in LLC-MK2 cells by Reed-Muench method. Influenza viruses were titrated by plaque formation assay (Si et al., 2020).

Stimulation of Cell Lines by Transfection

All RNAs and scrambled negative control dsRNA were synthesized by Integrated DNA Technologies, Inc. (IDT). Cells were seeded into 6-well plate at 3×10⁵ cells/well or 96-well plate at 10⁴ cells/well and cultured for 24 h before transfection. Transfection was performed using TransIT-X2 Dynamic Delivery System (Mirus) according to the manufacturer's instructions with some modifications. If not indicated otherwise, 6.8 μL of 10 μM RNA stock solution and 5 μL of transfection reagent were added in 200 μL Opti-MEM (Invitrogen) to make the transfection mixture. For transfection in 6-well plate, 200 μL of the transfection mixture was added to each well; for transfection in 96-well plate, 10 μL of the transfection mixture was added to each well. At indicated times after transfection, cell samples were collected and subjected to RNA-seq (Genewiz, Inc.), TMT Mass spectrometry, qRT-PCR, western blot, or Quanti-Luc assay (InvivoGen).

RNA-Seq and Gene Ontogeny Analysis

RNA-seq was processed by Genewiz using a standard RNA-seq package that includes polyA selection and sequencing on an Illumina HiSeq with 150-bp pair-ended reads. Sequence reads were trimmed to remove possible adapter sequences and nucleotides with poor quality using Trimmomatic v.0.36. The trimmed reads were mapped to the Homo sapiens GRCh38 reference genome using the STAR aligner v.2.5.2b. Unique gene hit counts were calculated by using feature Counts from the Subread package v.1.5.2 followed by differential expression analysis using DESeq2. Gene Ontology analysis was performed using DAVID (Huang da et al., 2009). Volcano plots and heat maps were generated using GraphPad Prism. Data for RNA-seq of A549 cells treated with Poly (I:C) was retrieved from Gene Expression Omnibus under the accession number GSE124144 (Burke et al., 2019).

Proteomics Analysis by Tandem Mass Tag Mass Spectrometry

Cells were harvested on ice. Cells pellets were syringe-lysed in 8 M urea and 200 mM EPPS pH 8.5 with protease inhibitor. BCA assay was performed to determine protein concentration of each sample. Samples were reduced in 5 mM TCEP, alkylated with 10 mM iodoacetamide, and quenched with 15 mM DTT. 100 μg protein was chloroform-methanol precipitated and re-suspended in 100 μL 200 mM EPPS pH 8.5. Protein was digested by Lys-C at a 1:100 protease-to-peptide ratio overnight at room temperature with gentle shaking. Trypsin was used for further digestion for 6 hours at 37° C. at the same ratio with Lys-C. After digestion, 30 μL acetonitrile (ACN) was added into each sample to 30% final volume. 200 μg TMT reagent (126, 127N, 127C, 128N, 128C, 129N, 129C, 130N, 130C) in 10 μL ACN was added to each sample. After 1 hour of labeling, 2 μL of each sample was combined, desalted, and analyzed using mass spectrometry. Total intensities were determined in each channel to calculate normalization factors. After quenching using 0.3% hydroxylamine, eleven samples were combined in 1:1 ratio of peptides based on normalization factors. The mixture was desalted by solid-phase extraction and fractionated with basic pH reversed phase (BPRP) high performance liquid chromatography (HPLC), collected onto a 96 six well plate and combined for 24 fractions in total. Twelve fractions were desalted and analyzed by liquid chromatography-tandem mass spectrometry (LC-MS/MS) (Navarrete-Perea et al., 2018).

Mass spectrometric data were collected on an Orbitrap Fusion Lumos mass spectrometer coupled to a Proxeon NanoLC-1200 UHPLC. The 100 μm capillary column was packed with 35 cm of Accucore 50 resin (2.6 m, 150 Å; ThermoFisher Scientific). The scan sequence began with an MS1 spectrum (Orbitrap analysis, resolution 120,000, 375-1500 Th, automatic gain control (AGC) target 4E5, maximum injection time 50 ms). SPS-MS3 analysis was used to reduce ion interference (Gygi et al., 2019; Paulo et al., 2016). The top ten precursors were then selected for MS2/MS3 analysis. MS2 analysis consisted of collision-induced dissociation (CID), quadrupole ion trap analysis, automatic gain control (AGC) 2E4, NCE (normalized collision energy) 35, q-value 0.25, maximum injection time 35 ms), and isolation window at 0.7. Following acquisition of each MS2 spectrum, an MS3 spectrum was collected in which multiple MS2 fragment ions are captured in the MS3 precursor population using isolation waveforms with multiple frequency notches. MS3 precursors were fragmented by HCD and analyzed using the Orbitrap (NCE 65, AGC 1.5E5, maximum injection time 120 ms, resolution was 50,000 at 400 Th).

Mass spectra were processed using a Sequest-based pipeline (Huttlin et al., 2010). Spectra were converted to mzXML using a modified version of ReAdW.exe. Database searching included all entries from the Human UniProt database (downloaded: 2014-02-04) This database was concatenated with one composed of all protein sequences in the reversed order. Searches were performed using a 50 ppm precursor ion tolerance for total protein level analysis. The product ion tolerance was set to 0.9 Da. TMT tags on lysine residues and peptide N termini (+229.163 Da) and carbamidomethylation of cysteine residues (+57.021 Da) were set as static modifications, while oxidation of methionine residues (+15.995 Da) was set as a variable modification.

Peptide-spectrum matches (PSMs) were adjusted to a 1% false discovery rate (FDR) (Elias and Gygi, 2007, 2010). PSM filtering was performed using a linear discriminant analysis (LDA), as described previously (Huttlin et al., 2010), while considering the following parameters: XCorr, ACn, missed cleavages, peptide length, charge state, and precursor mass accuracy. For TMT-based reporter ion quantitation, the summed signal-to-noise (S:N) ratio was extracted for each TMT channel and found the closest matching centroid to the expected mass of the TMT reporter ion. For protein-level comparisons, PSMs were identified, quantified, and collapsed to a 1% peptide false discovery rate (FDR) and then collapsed further to a final protein-level FDR of 1%, which resulted in a final peptide level FDR of <0.1%. Moreover, protein assembly was guided by principles of parsimony to produce the smallest set of proteins necessary to account for all observed peptides. Proteins were quantified by summing reporter ion counts across all matching PSMs, as described previously (Huttlin et al., 2010). PSMs with poor quality, MS3 spectra with TMT reporter summed signal-to-noise of less than 100, or having no MS3 spectra were excluded from quantification (McAlister et al., 2012). Each reporter ion channel was summed across all quantified proteins and normalized assuming equal protein loading of all tested samples.

qRT-PCR

Total RNA was extracted from cells using RNeasy Plus Mini Kit (QiaGen, Cat #74134) according to the manufacturer's instructions. cDNA was then synthesized using AMV reverse transcriptase kit (Promega) according to the manufacturer's instructions. To detect gene levels, quantitative real-time PCR was carried out using the GoTaq qPCR Master Mix kit (Promega) with 20 μL of reaction mixture containing gene-specific primers or the PrimePCR assay kit (Bio-Rad) according the manufacturers' instructions. The expression levels of target genes were normalized to GAPDH.

Antibodies and Western Blotting

The antibodies used in this study were anti-IRF3 (Abcam, ab68481), anti-IRF3 (Phospho S396) (Abcam, ab138449), anti-GAPDH (Abcam, ab9385), and Goat anti-Rabbit IgG H&L (HRP) (Abcam, ab205718). Cells were harvested and lysed in RIPA buffer (Thermo Scientific, Cat #89900) supplemented with Haltrm protease and phosphatase inhibitor cocktail (Thermo Scientific, Cat #78440) on ice. The cell lysates were subject to western blotting. GAPDH was used as a loading control.

Confocal Immunofluorescence Microscopy

Cells were rinsed with PBS, fixed with 4% paraformaldehyde (Alfa Aesar) for 30 min, permeabilized with 0.1% Triton X-100 (Sigma-Aldrich) in PBS (PBST) for 10 min, blocked with 10% goat serum (Life Technologies) in PBST for 1 h at room temperature, and incubated with anti-IRF3 (Phospho S396) (Abcam, ab138449) antibody diluted in blocking buffer (1% goat serum in PBST) overnight at 4° C., followed by incubation with Alexa Fluor 488 conjugated secondary antibody (Life Technologies) for 1 h at room temperature; nuclei were stained with DAPI (Invitrogen) after secondary antibody staining. Fluorescence imaging was carried out using a confocal laser-scanning microscope (SP5×MP DMI-6000, Germany) and image processing was done using Imaris software (Bitplane, Switzerland).

Surface Plasmon Resonance

The interactions between duplex RNA-1 and cellular RNA sensor molecules (RIG-I (Abcam, Cat #ab271486), MDA5 (Creative-Biomart, Cat #IFIH1-1252H), and TLR3 (Abcam, Cat #ab73825)) were analyzed by SPR with the Biacore T200 system (GE Healthcare) at 25° C. (Creative-Biolabs Inc.). RNA-1 conjugated to biotin at its 3′-terminus (synthesized by IDT Inc.) was immobilized on an SPR sensor chip, with final levels of ˜60 response units (RU). Various concentrations of the RNA sensors diluted in running buffer (10×HBS-EP+; GE Healthcare, Cat #BR100669) were injected as analytes at a flow rate of 30 μl/min, a contact time of 180 s, and a dissociation time of 300 s. The surface was regenerated with 2 M NaCl for 60 s. Data analysis was performed on the Biacore T200 computer with the Biacore T200 evaluation software.

Organ Chip Culture

Microfluidic two-channel Organ Chip devices and automated ZOE® instruments used to culture them were obtained from Emulate Inc (Boston, Mass., USA). Our methods for culturing human Lung Airway Chips (Si et al., 2020; Si et al., 2019) and Lung Alveolus Chips have been described previously. In this study, the Alveolus Chip method was slightly modified by coating the inner channels of the devices with 200 ug/ml Collagen IV (5022-5MG, Advanced Biomatrix) and 15 μg/ml of laminin (L4544-100UL, Sigma) at 37° C. overnight, and the next day (day 1) sequentially seeding primary human lung microvascular endothelial cells (Lonza, CC-2527, P5) and primary human lung alveolar epithelial cells (Cell Biologics, H-6053) in the bottom and top channels of the chip at a density of 8 and 1.6×10⁶ cells/ml, respectively, under static conditions. On day 2, the chips were inserted into Pods® (Emulate Inc.), placed within the ZOE® instrument, and the apical and basal channels were respectively perfused (60 μL/hr) with epithelial growth medium (Cell Biologics, H6621) and endothelial growth medium (Lonza, CC-3202). On day 5, 1 uM dexamethasone was added to the apical medium to enhance barrier function. On day 7, an air-liquid interface (ALI) was introduced into the epithelial channel by removing all medium from this channel while continuing to feed all cells through the medium perfused through the lower vascular channel, and this medium was changed to EGM-2MV with 0.5% FBS on day 9. Two days later, the ZOE® instrument was used to apply cyclic (0.25 Hz) 5% mechanical strain to the engineered alveolar-capillary interface to mimic lung breathing on-chip. RNAs were transfected on Day 15.

RNA Transfection in Human Lung Airway and Alveolus Chips

Human Airway or Alveolus Chips were transfected with duplex RNAs by adding the RNA and transfection reagent (Lipofectamine RNAiMAX) mixture into the apical and basal channels of the Organ Chips and incubating for 6 h at 37° C. under static conditions before reestablishing an ALI. Tissues cultured on-chip were collected by RNeasy Micro Kit (QiaGen) at 48 h post-transfection by first introducing 100 ul lysis buffer into the apical channel to lyse epithelial cells and then 100 ul into the basal channel to lyse endothelial cells. Lysates were subjected to qPCR analysis of IFN-β gene expression.

Native SARS-CoV-2 Infection and Inhibition by RNA Treatment

ACE2-expressing A549 cells (a gift from Brad Rosenberg) were transfected with indicated RNAs. 24 h post-transfection, the transfected ACE2-A549 cells were infected with SARS-CoV-2 (MOI=0.05) for 48 hours. Cells were harvested in Trizol (Invitrogen) and total RNA was isolated and DNAse-I treated using Zymo RNA Miniprep Kit according to the manufacturer's protocol. qRT-PCR for α-tubulin (Forward: 5′-GCCTGGACCACAAGTTTGAC-3′ (SEQ ID NO: 44); Reverse: 3′-TGAAATTCTGGGAGCATGAC-5′ (SEQ ID NO: 45)) and SARS-CoV-2 N mRNA (Forward: 5′-CTCTTGTAGATCTGTTCTCTAAACGAAC-3′ (SEQ ID NO: 46); Reverse: 3′-GGTCCACCAAACGTAATGCG-5′ (SEQ ID NO: 47)) were performed using KAPA SYBR FAST ONE-STEP qRT-PCR kits (Roche) according to manufacturer's instructions on a Lightcycler 480 Instrument-II (Roche).

Native SARS-CoV-1 and MERS-CoV Infection and Inhibition by RNA Treatment

Vero E6 cells (ATCC #CRL 1586) were cultured in DMEM (Quality Biological), supplemented with 10% (v/v) fetal bovine serum (Sigma), 1% (v/v) penicillin/streptomycin (Gemini Bio-products) and 1% (v/v) L-glutamine (2 mM final concentration, Gibco). Cells were maintained at 37° C. (5% CO₂). Vero E6 cells were plated at 1.5×10⁵ cells per well in a six well plate two days prior to transfection. The RNA-1, RNA-2, and scrambled control RNA were transfected into each well using the Transit X2 delivery system (MIRUS; MIR6003) in OptiMEM (Gibco 31985-070). SARS-CoV (Urbani strain, BEI #NR-18925) and MERS-CoV (Jordan strain, provided by NIH) were added at MOI 0.01. At 72 hours post infection, medium was collected and used for a plaque assay to quantify PFU/mL of virus.

Hamster Efficacy Studies

The methods for carrying out efficacy studies in Golden hamsters using native SARS-CoV-2 Isolate USA-WA1/2020 (NR-52281) were as described previously (13). In the prevention studies, RNA-1 diluted in PBS was administered intranasally beginning 1 day prior to intranasal administration of SARS-CoV-2 virus (10² PFU of passage 3 virus in 100 μl of PBS) and daily for 2 additional days. In the treatment experiments, RNA-1 diluted in 5% glucose containing in vivo-jetPEI® Delivery Reagent (Genesee Scientific Cat #: 55-202G; 20 ug in 50 uL) was administered intranasally daily for 2 days beginning 1 day after intranasal administration of SARS-CoV-2 virus (10³ PFU). In all experiments, animals were sacrificed and lungs harvested for analysis 1 day after the last treatment was administered. Animals were anesthetized by intraperitoneal injection of 100 μl of ketamine and xylazine (3:1) and provided thermal support while unconscious, and whole lungs were harvested for analysis by RT-qPCR or plaque assay.

Lung RNA was extracted by phenol chloroform extraction and DNase treatment using a DNA-Free™ DNA removal kit (Invitrogen), and RT-qPCR was performed using KAPA SYBR FAST qPCR Master Mix Kit (Kapa Biosystems) on a LightCycler 480 Instrument II (Roche) for subgenomic nucleocapsid (N) RNA (sgRNA) and actin using the following primers: Actin forward primer: 5′-CCAAGGCCAACCGTGAAAAG-3′ (SEQ ID NO: 48), Actin reverse primer 5′-ATGGCTACGTACATGGCTGG-3′ (SEQ ID NO: 49), N sgRNA forward primer: 5′-CTCTTGTAGATCTGTTCTCTAAACGAAC-3′ (SEQ ID NO: 46), N sgRNA reverse primer: 5′-GGTCCACCAAACGTAATGCG-3′ (SEQ ID NO: 50). Relative sgRNA levels were quantified by normalizing sgRNA to actin expression.

Quantification and Statistical Analysis

All data are expressed as mean±standard deviation (SD). N represents biological replicates. Statistical significance of differences in the in vitro experiments was determined by employing the paired two-tailed Student t-test when comparing the difference between two groups and one-way ANOVA with multiple comparison when comparing the samples among groups with more than two samples. For in vivo experiments, an unpaired one-tailed Student t-test was used to estimate significance of viral load inhibition by RNA-1. For all experiments, differences were considered statistically significant for p<0.05 (*, p<0.05; **, p<0.01; ***, p<0.001; n.s., not significant).

### REFERENCES

1) D. Blanco-Melo et al., Imbalanced Host Response to SARS-CoV-2 Drives Development of COVID-19. Cell 181, 1036-1045 e1039 (2020).
2) L. Si et al., Human organs-on-chips as tools for repurposing approved drugs as potential influenza and COVID19 therapeutics in viral pandemics. bioRxiv doi:10.1101/2020.04.13.039917. (2020).
3) W. Huang da, B. T. Sherman, R. A. Lempicki, Systematic and integrative analysis of large gene lists using DAVID bioinformatics resources. Nat Protoc 4, 44-57 (2009).
4) J. M. Burke, S. L. Moon, T. Matheny, R. Parker, RNase L Reprograms Translation by Widespread mRNA Turnover Escaped by Antiviral mRNAs. Mol Cell 75, 1203-1217 e1205 (2019).
5) J. Navarrete-Perea, Q. Yu, S. P. Gygi, J. A. Paulo, Streamlined Tandem Mass Tag (SL-TMT) Protocol: An Efficient Strategy for Quantitative (Phospho)proteome Profiling Using Tandem Mass Tag-Synchronous Precursor Selection-MS3. J Proteome Res 17, 2226-2236 (2018).
6) J. P. Gygi et al., Web-Based Search Tool for Visualizing Instrument Performance Using the Triple Knockout (TKO) Proteome Standard. J Proteome Res 18, 687693 (2019).
7) J. A. Paulo, J. D. O'Connell, S. P. Gygi, A Triple Knockout (TKO) Proteomics Standard for Diagnosing Ion Interference in Isobaric Labeling Experiments. J Am Soc Mass Spectrom 27, 1620-1625 (2016).

### Example 4: Novel Duplex RNAs Inhibit Infection by Multiple Influenza Strains

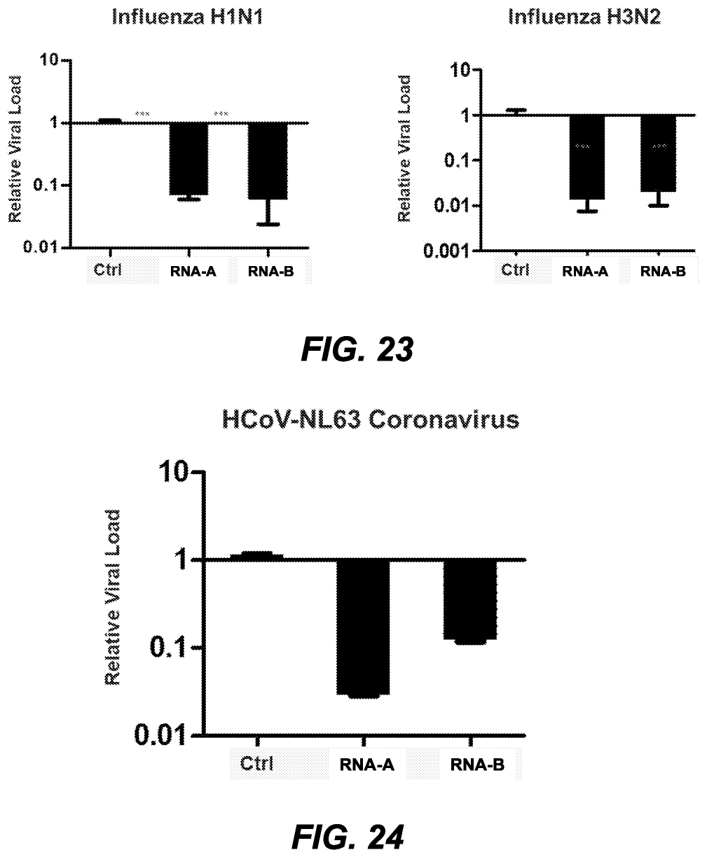

The duplex RNAs described herein were tested for their ability to inhibit infection in human and primate cell cultures. The duplex RNAs described herein provided greater than 95% inhibition of influenza infection in human lung epithelial cells (FIG. 23). When tested on monkey kidney cells infected with common cold coronavirus, HCoV-NL63, the RNA duplexes described herein also inhibited greater than 95% of coronavirus infection in the monkey kidney cells (FIG. 24).

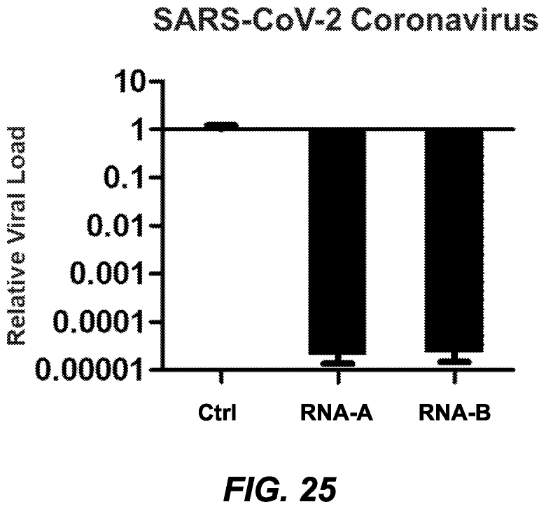

Most notably, the RNA duplexes described herein inhibited SARS-CoV-2 virus infection in ACE2-overexpressing Human Lung Epithelial Cells (FIG. 25).

Method—Cell Culture and Virus: Vero E6 cells (ATCC #CRL 1586) were cultured in DMEM (Quality Biological®), supplemented with 10% (v/v) fetal bovine serum (Sigma), 1% (v/v) penicillin/streptomycin (Gemini Bio-Products®) and 1% (v/v) L-glutamine (2 mM final concentration, Gibco®). Cells were maintained at 37° C. (5% C02). Vero E6 cells were plated at 1.5E5 cells per well in a six well plate two days prior to transfection. The RNA-A, RNA-B and scrambled control RNA were transfected into each well of a six-well plate using the Transit X2™ delivery system (MIRUS®; MIR6003) in OptiMEM (Gibco® 31985-070). SARS-CoV (Urbani strain, BEI #NR-18925) and MERS-CoV (Jordan strain, provided by NIH) were added at MOI 0.01. At 72 hours post infection, media was collected and used for a plaque assay to quantify pfu/ml of virus (e.g., Coleman C M, Frieman M B. 2015. Growth and Quantification of MERS-CoV Infection. Curr Protoc Microbiol 37:15E.2.1-15E.2.9.).

### Example 5: Novel RNA Duplexes Inhibit SARS-COV-2 Infection In Vivo

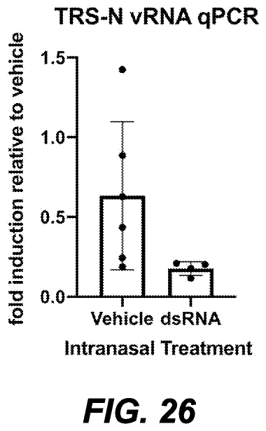

The RNA duplexes described herein were tested in vivo by pulmonary administration in hamsters infected with SARS-CoV2. Induction of interferon Type I by duplex RNA administered on day −1, 0, and +1 of infection is sufficient to significantly reduce viral load in the animals (FIG. 26).

In summary, dsRNAs described herein when delivered to lung airways can produce higher IFN responses locally than IFN protein formulations that are injected systemically. Furthermore, the dsRNAs do not produce generalized inflammatory responses seen with other immunostimulatory RNAs, minimizing toxicity. The dsRNAs described herein can be used for both prophylaxis as well as treatment in COVID-19 and influenza infections, among others.

1. An immunostimulatory oligonucleotide duplex comprising SEQ ID NO:1 at a 5′ end.

2. The immunostimulatory oligonucleotide duplex of claim 1, wherein the oligonucleotide duplex is RNA.

3. The immunostimulatory oligonucleotide duplex of claim 1 or 2, wherein the oligonucleotide duplex comprises a 5′-monophosphate group

4. The immunostimulatory oligonucleotide duplex of any of claims 1-3, wherein the oligonucleotide duplex is at least 20 nucleobases in length.

5. The immunostimulatory oligonucleotide duplex of claim 1, wherein the oligonucleotide duplex is double stranded RNA.

6. The immunostimulatory oligonucleotide duplex of claim 1, wherein the oligonucleotide duplex is sufficient to induce interferon (IFN) production in a cell contacted with the duplex.

7. The method of claim 6, wherein the IFN production is type I IFN production.

8. The immunostimulatory oligonucleotide duplex of claim 1, wherein the oligonucleotide duplex activates the RIG-I-IRF3 pathway.

9. The immunostimulatory oligonucleotide duplex of claim 1, wherein the oligonucleotide duplex reduces a viral titer or viral load in a cell or population of cells contacted with the duplex.

10. The immunostimulatory oligonucleotide duplex of claim 1, wherein the oligonucleotide duplex increases STAT1 and STAT2 in a cell contacted by the duplex.

11. A method of inducing an anti-viral response is a subject, the method comprising administering to a subject in need thereof an immunostimulatory oligonucleotide duplex of any of claims 1-10.

12. A method of treating a viral infection in a subject, the method comprising administering to a subject in need thereof an immunostimulatory oligonucleotide duplex of any of claims 1-10.

13. The method of claim 11 or 12, wherein the subject in need thereof has a viral infection, or is at risk of having a viral infection.

14. The method of claim 11 or 12, further comprising, prior to administering, a step of diagnosing the subject as having a viral infection or being at risk of having a viral infection.

15. The method of claim 11 or 12, further comprising, prior to administering, a step of receiving results of an assay that diagnoses the subject as having a viral infection or as being at risk of having a viral infection.

16. The method of any of claims 11-15, wherein the viral infection is caused by a virus selected from the group consisting of: John Cunningham virus, measles virus, Lymphocytic choriomeningitis virus, arbovirus, rabies virus, rhinovirus, parainfluenza virus, respiratory syncytial virus, herpes simplex virus, herpes simplex type 1, herpes simplex type 2, human herpesvirus 6, adenovirus, cytomegalovirus, Epstein-Barr virus, mumps virus, influenza virus type A, influenza virus type B, coronavirus, SARS coronavirus, SARS-CoV-2 virus, coxsackie A virus, coxsackie B virus, poliovirus, HTLV-1, hepatitis virus types A, B, C, D, and E, varicella zoster virus, smallpox virus, molluscum contagiosum, human papillomavirus, parvovirus B19, rubella virus, human immunodeficiency virus, rotavirus, norovirus, astrovirus, ebola virus, Marburg virus, dengue virus (DENV), and Zika virus.

17. The method of any of claims 11-16, wherein the viral infection is an infection of a tissue selected from the group consisting of central nervous system tissue, eye tissue, upper respiratory system tissue, lower respiratory system tissue, lung tissue, kidney tissue, bladder tissue, spleen tissue, cardiac tissue, gastrointestinal tissue, epidermal tissue, reproductive tissue, nasal cavity tissue, larynx tissue, trachea tissue, bronchi tissue, oral cavity tissue, blood tissue, and muscle tissue.

18. The method of any of claims 11-17, wherein the administration is systemic.

19. The method of any of claims 11-17, wherein the administration is local at a site of viral infection.

20. The method of any of claims 11-19, further comprising administering at least one additional therapeutic.

21. The method of claim 20, wherein the at least one additional therapeutic is an anti-viral therapeutic.

22. A method of treating an influenza infection in a subject, the method comprising administering to a subject having an influenza infection an immunostimulatory oligonucleotide duplex of any of claims 1-10.

23. The method of claim 22, wherein the influenza infection is an influenza A infection, or an influenza B infection.

24. The method of claim 22 or 23, further comprising administering at least one additional anti-viral therapeutic.

25. A method of treating a coronavirus disease in a subject, the method comprising administering to a subject having a coronavirus disease an immunostimulatory oligonucleotide duplex of any of claims 1-10.

26. The method of claim 25, wherein the coronavirus disease is COVID-19.

27. The method of claim 25 or 26, further comprising administering at least one additional anti-viral therapeutic.

28. The method of claim 25 or 26, further comprising administering plasma obtained from a subject that has recovered from the coronavirus disease.

29. A method of increasing the efficacy of an anti-viral therapeutic, the method comprising administering an immunostimulatory oligonucleotide duplex of any of claims 1-10 and at least one anti-viral therapeutic.

30. The method of claim 29, wherein the anti-viral therapeutic is selected from the group consisting of: Abacavir, Acyclovir (Aciclovir), Adefovir, Amantadine, Ampligen, Amprenavir (Agenerase), Amodiaquine, Apilimod, Arbidol, Atazanavir, Atripla, Atovaquone, Balavir, Baloxavir marboxil (Xofluza®), Biktarvy Boceprevir (Victrelis®), Cidofovir, Clofazimine, Clomifene, Clofazamine, Cobicistat (Tybost®), Combivir (fixed dose drug), Daclatasvir (Daklinza®), Darunavir, Delavirdine, Descovy, Didanosine, Docosanol, Dolutegravir, Doravirine (Pifeltro®), Ecoliever, Edoxudine, Efavirenz, Elvitegravir, Emtricitabine, Enfuvirtide, Entecavir, Etravirine (Intelence®), Famciclovir, Favipiravir, Fenofibrate, Fomivirsen, Fosamprenavir, Foscarnet, Fosfonet, Fusion inhibitor, Ganciclovir (Cytovene®), Ibacitabine, Ibalizumab (Trogarzo®), Idoxuridine, Imiquimod, Imunovir, Indinavir, Inosine, Integrase inhibitor, Interferon type I, Interferon type II, Interferon type III, Interferon, Ivermectin, Lamivudine, Lasalocid, Letermovir (Prevymis®), Lopinavir, Loviride, Mannose Binding Lectin, Maraviroc, Methisazone, Moroxydine, Nafamostat, Nelfinavir, Nevirapine, Nexavir®, Nilotinib, Nitazoxanide, Norvir, Nucleoside analogues, Oseltamivir (Tamiflu®), Pazopanib, Peginterferon alfa-2a, Peginterferon alfa-2b, Penciclovir, Peramivir (Rapivab®), Pleconaril, Podophyllotoxin, Protease inhibitor (pharmacology), Pyonaridine, Pyramidine, Raltegravir, Remdesivir, Reverse transcriptase inhibitor, Ribavirin, Rilpivirine (Edurant®), Rimantadine, Ritonavir, Saquinavir, Simeprevir (Olysio®), Sofosbuvir, Stavudine, Synergistic enhancer (antiretroviral), Tafenoquine, Telaprevir, Telbivudine (Tyzeka®), Tenofovir alafenamide, Tenofovir disoproxil, Tenofovir, Toremifene, Tipranavir, Trifluridine, Trizivir, Tromantadine, Truvada, Valaciclovir (Valtrex), Valganciclovir, Vermurafenib, Venetoclax, Vicriviroc, Vidarabine, Viramidine, Zalcitabine, Zanamivir (Relenza®), and Zidovudine.

31. The method of claim 29 or 30, wherein the immunostimulatory oligonucleotide duplex and the at least one antiviral therapeutic are administered at substantially the same time.

32. The method of claim 29 or 30, wherein the immunostimulatory oligonucleotide duplex and the at least one antiviral therapeutic are administered at different time points.

33. A pharmaceutical composition comprising an immunostimulatory oligonucleotide duplex of any of claims 1-10 and a pharmaceutically acceptable carrier.

34. A pharmaceutical composition comprising an immunostimulatory oligonucleotide duplex of any of claims 1-10 and at least one anti-viral therapeutic.

35. The composition of claim 33 or 34, wherein the composition is formulated for airway administration.

36. The composition of claim 35, wherein the composition is formulated for aerosol administration, nebulizer administration, or tracheal lavage administration.

37. A method of inducing interferon (IFN) production, the method comprising administering to a subject in need thereof an immunostimulatory oligonucleotide duplex of any of claims 1-10, or a pharmaceutical composition of any of claims 33-36, whereby IFN production is increased following administration.

38. The method of claim 37, wherein IFN production is the production of type I IFN, type II IFN, or type III IFN.

39. The method of claim 37 or 38, wherein IFN production is the production of type I IFN.

40. The method of any of claims 37-39, wherein the type I IFN is IFN-α, IFN-β, IFN-ε, IFN-κ or IFN-ω.

41. The method of claim 38, wherein the type II IFN is IFN-γ.

42. The method of claim 37, wherein increased IFN production increases cellular resistance to a viral infection.

43. A method of treating an IFN-associated disease, the method comprising administering to a subject in need thereof an immunostimulatory oligonucleotide duplex of any of claims 1-10.

44. The method of claim 43, wherein the subject in need thereof has an IFN-associated disease, or is at risk of having an IFN-associated disease.

45. The method of claim 43, further comprising, prior to administering, a step of diagnosing a subject as having an IFN-associated disease or at risk of having an IFN-associated disease.

46. The method of claim 43, further comprising, prior to administering, receiving the results of an assay that diagnoses a subject as having an IFN-associated disease or at risk of an IFN-associated disease.

47. The method of claim 43, wherein the IFN-associated disease is a disease involving reduced IFN levels as compared to a reference level.

48. The method of claim 43, wherein the IFN-associated disease is a disease involving reduced Type I IFN levels as compared to a reference level.

49. The method of any of claims 43-48, wherein the IFN-associated disease is selected from the group consisting of a viral infectious disease, a bacterial infectious disease, a fungal infectious disease, a parasitic infectious disease, cancer, and an autoimmune disease.

50. The method of any of claims 43-49, further comprising administering at least one additional therapeutic.

51. The method of claim 50, wherein the at least one additional therapeutic is an anti-viral therapeutic, an anti-bacterial therapeutic, an anti-fungal therapeutic, an anti-parasitic therapeutic, an anti-cancer therapeutic, or an anti-autoimmune therapeutic.

52. A composition comprising an immunostimulatory oligonucleotide duplex of any of claims 1-10 and at least one anti-bacterial therapeutic.

53. A composition comprising an immunostimulatory oligonucleotide duplex of any of claims 1-10 and at least one anti-fungal therapeutic.

54. A composition comprising an immunostimulatory oligonucleotide duplex of any of claims 1-10 and at least one anti-parasitic therapeutic.

55. A composition comprising an immunostimulatory oligonucleotide duplex of any of claims 1-10 and at least one anti-cancer therapeutic.

56. A composition comprising the immunostimulatory oligonucleotide duplex of any of claims 1-10 and at least one anti-autoimmune therapeutic.

57. The composition of any of claims 52-56, further comprising a pharmaceutically acceptable carrier.

58. An immunostimulatory oligonucleotide duplex comprising SEQ ID NO:1 at a 5′ end, conjugated to an antigen or vaccine.

59. A composition comprising an immunostimulatory oligonucleotide duplex of claim 58.

60. A composition comprising an immunostimulatory oligonucleotide duplex of any of claims 1-10 and a vaccine.

61. A composition comprising an immunostimulatory oligonucleotide duplex of any of claims 1-10 and a nanoparticle.

62. A nanoparticle comprising an immunostimulatory oligonucleotide duplex of any of claims 1-10.

63. A composition comprising an immunostimulatory oligonucleotide duplex of claim 58 and a nanoparticle.

64. A nanoparticle comprising an immunostimulatory oligonucleotide duplex of claim 58.

65. The composition of claims 59-64, further comprising a pharmaceutically acceptable carrier.

66. A method of vaccinating, the method comprising administering to a subject in need thereof
a. an immunostimulatory oligonucleotide duplex of claim 58;
b. a composition of any of claim 59-64; or
c. an immunostimulatory oligonucleotide duplex of any of claims 1-10, and a vaccine.

67. A method of increasing the efficacy of a vaccine, the method comprising administering to a subject in need thereof
a. the immunostimulatory oligonucleotide duplex of claim 58;
b. a composition of any of claims 59-64; or
c. an immunostimulatory oligonucleotide duplex of any of claims 1-10, and a vaccine.

68. The composition of claim 33 or 34, wherein the composition is formulated for intravenous, intramuscular, intraperitoneal, subcutaneous, or intrathecal administration.
